(19) United States
(12) Patent Application Publication
(10) Pub. No.: US 2023/0340489 A1
(43) Pub. Date: Oct. 26, 2023

(54) COMPOSITIONS AND METHODS FOR TREATING AMYOTROPHIC LATERAL SCLEROSIS (ALS) WITH AAV-MIR-SOD1

(71) Applicant: Biogen MA Inc., Cambridge, MA (US)

(72) Inventors: Shih-Ching Lo, Boston, MA (US); Alexander McCampbell, Andover, MA (US); Maria Zavodszky, Arlington, MA (US); Edward Guilmette, Franklin, MA (US); Barret Pfeiffer, Boston, MA (US)

(21) Appl. No.: 18/026,772

(22) PCT Filed: Sep. 15, 2021
(86) PCT No.: PCT/US2021/050492

§ 371 Date: Mar. 16, 2023

Related U.S. Application Data
(60) Provisional application No. 63/079,459, filed on Sep. 16, 2020.

(51) Int. Cl. C12N 15/113; C12N 15/86; A61P 25/28; A61K 48/00

(52) U.S. Cl. CPC C12N 15/1137; C12N 15/86; A61P 25/28; A61K 48/0058; C12Y 115/01001

(57) ABSTRACT

The present disclosure provides compositions and methods for treating amyotrophic lateral sclerosis (ALS). Among other things, the present disclosure provides inhibitory nucleic acids that inhibit the expression of genes that cause or are implicated in ALS pathogenesis. The present disclosure further provides recombinant adeno-associated virus (rAAV) vectors comprising inhibitory nucleic acids that inhibit the expression of genes that cause or are implicated in ALS pathogenesis.

### CROSS REFERENCE TO RELATED APPLICATIONS

This application claims priority to and the benefit of U.S. Provisional Patent Application No. 63/079,459 filed Sep. 16, 2020, the entirety of which is incorporated herein by reference.

### BACKGROUND

ALS, or amyotrophic lateral sclerosis, is a progressive neurodegenerative disease that affects nerve cells in the brain and the spinal cord. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and some people experience pain. Most eventually lose the ability to walk, use their hands, speak, swallow, and breathe. There is no known cure for ALS. There are currently only four drugs approved by the U.S. FDA to treat ALS (Riluzole, Nuedexta, Radicava, and Tiglutik). There is therefore a need in the art for therapeutic modalities to treat ALS.

### SUMMARY

The present disclosure provides certain insights in the development of compositions and methods for treatment of ALS. The present disclosure provides, among other things, compositions and methods for treating amyotrophic lateral sclerosis (ALS). In some embodiments, the present disclosure provides inhibitory nucleic acids that inhibit the expression of genes that cause or are implicated in ALS pathogenesis. In some embodiments, the present disclosure provides recombinant adeno-associated virus (rAAV) vectors comprising inhibitory nucleic acids that inhibit the expression of genes that cause or are implicated in ALS pathogenesis. In some embodiments, the present disclosure provides compositions and methods for treating ALS that include rAAV vectors comprising one or more miRNAs that inhibit SOD1 expression. In some embodiments, the present disclosure provides compositions and methods for treating ALS that include rAAV vectors comprising at least two or more miRNAs that inhibit SOD1 expression. In some embodiments, miRNAs of the present disclosure are modified and/or engineered as compared to wild-type miRNAs. In some embodiments, inhibitory nucleic acids of the present disclosure target SOD1 mutants associated ALS disease pathogenesis.

The present disclosure further provides compositions and methods for treating ALS that exhibit reduced toxicity and/or immunoreactivity in a subject compared to compositions and methods known in the art. In some embodiments, methods that exhibit reduced toxicity and/or immunoreactivity in a subject comprise administration of rAAV vectors comprising inhibitory nucleic acids that inhibit expression of genes that cause or are implicated in ALS pathogenesis. Administration of compositions of the present disclosure may be by any method available to those skilled in the art. In some embodiments, administration maybe intrathecal-lumbar puncture (LP). In some embodiments, administration may be intrathecal-intracisterna magna (ICM). In some embodiments, administration may be subpial injection, three-point injection of LP, ICM, and intracerebral ventricular (ICV), catheterized ICM, or any combination thereof. In some embodiments, administration may be conducted by any combination of administration methods described herein. In some preferred embodiments of the present disclosure, methods of treating ALS that exhibit reduced toxicity and/or immunoreactivity in a subject comprise administration of inhibitory nucleic acids (e.g., in the form of rAAV) by intrathecal injection.

In some embodiments, the present disclosure provides a recombinant adeno-associated virus (rAAV) vector comprising: a) a modified AAV genome comprising: (i) a promoter; and (ii) at least two or more different miRNA sequences; and b) a capsid; wherein each of the two or more miRNA sequences comprise a guide strand sequence that targets SOD1, and a scaffold sequence and wherein each of the two or more miRNA sequences are operably linked to the promoter.

In some embodiments, at least two miRNA sequences comprise at least one guide strand sequence that shares at least 80% sequence identity to SEQ ID NO: 2 and at least one guide strand sequence that shares at least 80% sequence identity to SEQ ID NO: 5.

In some embodiments, at least two miRNA sequences comprise at least one guide strand sequence that includes at least 5 (e.g., at least 10, at least 15, at least 20, etc.) contiguous nucleotides with reference to SEQ ID NO: 2 and at least one guide strand sequence that includes at least 5 (e.g., at least 10, at least 15, at least 20, etc.) contiguous nucleotides with reference to SEQ ID NO: 5.

In some embodiments, at least two miRNA sequences comprise at least one guide strand sequence comprising SEQ ID NO: 2 and at least one guide strand sequence comprising SEQ ID NO: 5.

In some embodiments, at least one miRNA sequence comprises a scaffold sequence that shares at least 80% sequence identity to SEQ ID NO: 16.

In some embodiments, at least one miRNA sequence comprises a scaffold sequence comprising SEQ ID NO: 16.

In some embodiments, at least one miRNA sequence comprises a scaffold sequence that shares at least 80% sequence identity to SEQ ID NO: 18.

In some embodiments, at least one miRNA sequence comprises a scaffold sequence comprising SEQ ID NO: 18.

In some embodiments, at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 5.

In some embodiments, at least two miRNA sequences comprise one miRNA sequence with a guide strand sequence comprising SEQ ID NO: 2, and one miRNA sequence with a guide strand sequence comprising SEQ ID NO: 5 and a scaffold sequence comprising SEQ ID NO: 18.

In some embodiments, at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 5 and a scaffold sequence comprising SEQ ID NO: 18.

In some embodiments, at least two miRNA sequences comprise at least one guide strand sequence that shares at least 80% sequence identity to SEQ ID NO: 2 and at least one guide strand sequence that shares at least 80% sequences identity to SEQ ID NO: 7.

In some embodiments, at least two miRNA sequences comprise at least one guide strand sequence that includes at least 5 (e.g., at least 10, at least 15, at least 20, etc.) contiguous nucleotides with reference to SEQ ID NO: 2 and at least one guide strand sequence that includes at least 5 (e.g., at least 10, at least 15, at least 20, etc.) contiguous nucleotides with reference to SEQ ID NO: 7.

In some embodiments, at least two miRNA sequences comprise at least one guide strand sequence comprising SEQ ID NO: 2 and at least one guide strand sequence comprising SEQ ID NO: 7.

In some embodiments, at least one miRNA sequence comprises a scaffold sequence that shares at least 80% sequence identity to SEQ ID NO: 16.

In some embodiments, at least one miRNA sequence comprises a scaffold sequence comprising SEQ ID NO: 16.

In some embodiments, at least one miRNA sequence comprises a scaffold sequence that shares at least 80% sequence identity to SEQ ID NO: 17.

In some embodiments, at least one miRNA sequence comprises a scaffold sequence comprising SEQ ID NO: 17.

In some embodiments, at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 7.

In some embodiments, at least two miRNA sequences comprise one miRNA sequence with a guide strand sequence comprising SEQ ID NO: 2, and one miRNA sequence with a guide strand sequence comprising SEQ ID NO: 7 and a scaffold sequence comprising SEQ ID NO: 17.

In some embodiments, at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 7 and a scaffold sequence comprising SEQ ID NO: 17.

In some embodiments, two miRNA sequences comprise at least one guide strand sequence that shares at least 80% sequence identity to SEQ ID NO: 5 and at least one guide strand sequence that shares at least 80% identity to SEQ ID NO: 7.

In some embodiments, at least two miRNA sequences comprise at least one guide strand sequence that includes at least 5 (e.g., at least 10, at least 15, at least 20, etc.) contiguous nucleotides with reference to SEQ ID NO: 5 and at least one guide strand sequence that includes at least 5 (e.g., at least 10, at least 15, at least 20, etc.) contiguous nucleotides with reference to SEQ ID NO: 7.

In some embodiments, at least two miRNA sequences comprise at least one guide strand sequence comprising SEQ ID NO: 5 and at least one guide strand sequence comprising SEQ ID NO: 7.

In some embodiments, at least one miRNA sequence comprises a scaffold sequence that shares at least 80% sequence identity to SEQ ID NO: 18.

In some embodiments, at least one miRNA sequence comprises a scaffold sequence comprising SEQ ID NO: 18.

In some embodiments, at least one miRNA sequence comprises a scaffold sequence that shares at least 80% sequence identity to SEQ ID NO: 16.

In some embodiments, at least one miRNA sequence comprises a scaffold sequence comprising SEQ ID NO: 16.

In some embodiments, at least two miRNA sequences comprise one miRNA sequence with a guide strand sequence comprising SEQ ID NO: 5 and a scaffold sequence comprising SEQ ID NO: 18, and one miRNA sequence with a guide strand sequence comprising SEQ ID NO: 7.

In some embodiments, at least two miRNA sequences comprise one miRNA sequence with a guide strand sequence comprising SEQ ID NO: 5, and one miRNA sequence with a guide strand sequence comprising SEQ ID NO: 7 and a scaffold sequence comprising SEQ ID NO: 16.

In some embodiments, at least two miRNA sequences comprise one miRNA sequence with a guide strand sequence comprising SEQ ID NO: 5 and a scaffold sequence comprising SEQ ID NO: 18, and one miRNA sequence with a guide strand sequence comprising SEQ ID NO: 7 and a scaffold sequence comprising SEQ ID NO: 16.

In some embodiments, a modified AAV genome comprises at least three miRNA guide sequences.

In some embodiments, at least three miRNA guide sequences comprise at least one guide strand sequence that shares at least 80% sequence identity to SEQ ID NO: 2, at least one guide strand sequence that shares at least 80% identity to SEQ ID NO: 5, and at least one guide strand sequence that shares at least 80% identity to SEQ ID NO: 7.

In some embodiments, at least three miRNA guide sequences comprise at least one guide strand sequence that includes at least 5 (e.g., at least 10, at least 15, at least 20, etc.) contiguous nucleotides with reference to SEQ ID NO: 2, at least one guide strand sequence that includes at least 5 (e.g., at least 10, at least 15, at least 20, etc.) contiguous nucleotides with reference to SEQ ID NO: 5, and at least one guide strand sequence that includes at least 5 (e.g., at least 10, at least 15, at least 20, etc.) contiguous nucleotides with reference to SEQ ID NO: 7.

In some embodiments, at least three miRNA guide sequences comprise at least one guide strand sequence comprising SEQ ID NO: 2, at least one guide strand sequence comprising SEQ ID NO: 5, and at least one guide strand sequence comprising SEQ ID NO: 7.

In some embodiments, at least one miRNA sequence comprises a scaffold sequence that shares at least 80% sequence identity to SEQ ID NO: 16. In some embodiments, at least one miRNA sequence comprises a scaffold sequence comprising SEQ ID NO: 16.

In some embodiments, at least one miRNA sequence comprises a scaffold sequence that shares at least 80% sequence identity to SEQ ID NO: 18. In some embodiments, at least one miRNA sequence comprises a scaffold sequence comprising SEQ ID NO: 18.

In some embodiments, at least three miRNA sequence comprise at least one miRNA sequence with a guide strand sequence comprising SEQ ID NO: 2 and a scaffold sequence that shares at least 80% sequence identity to SEQ ID NO: 16, and at least one miRNA sequence with a guide strand sequence comprising SEQ ID NO: 5 and a scaffold sequence that shares at least 80% sequence identity to SEQ ID NO: 18.

In some embodiments, at least three miRNA sequences comprise at least one miRNA sequence with a guide strand sequence comprising SEQ ID NO: 2 and a scaffold sequence comprising SEQ ID NO: 16, and at least one miRNA sequence with a guide strand sequence comprising SEQ ID NO: 5 and a scaffold sequence comprising SEQ ID NO: 18.

In some embodiments, at least three miRNA sequences comprise at least one miRNA sequence with a guide strand sequence comprising SEQ ID NO: 2 and a scaffold sequence that shares at least 80% sequence identity to SEQ ID NO: 16, and at least one miRNA sequence with a guide strand sequence comprising 7 and a scaffold sequence that shares at least 80% sequence identity to SEQ ID NO: 17.

In some embodiments, at least three miRNA sequences comprise at least one miRNA sequence with a guide strand sequence comprising SEQ ID NO: 2 and a scaffold sequence comprising SEQ ID NO: 16, and at least one miRNA sequence with a guide strand sequence comprising SEQ ID NO: 7 and a scaffold sequence comprising SEQ ID NO: 17.

In some embodiments, at least three miRNA sequences comprise at least one miRNA sequence with a guide strand sequence comprising SEQ ID NO: 5 and a scaffold sequence that shares at least 80% sequence identity to SEQ ID NO: 18, and at least one miRNA sequence with a guide strand sequence comprising SEQ ID NO: 7 and a scaffold sequence that shares at least 80% sequence identity to SEQ ID NO: 16.

In some embodiments, at least three miRNA sequences comprise at least one miRNA sequence with a guide strand sequence comprising SEQ ID NO: 5 and a scaffold sequence comprising SEQ ID NO: 18, and at least one miRNA sequence with a guide strand sequence comprising SEQ ID NO: 7 and a scaffold sequence comprising SEQ ID NO: 16.

In some embodiments, at least three miRNA sequences comprise at least one miRNA sequence with a guide strand sequence comprising SEQ ID NO: 5 and a scaffold sequence that shares at least 80% sequence identity to SEQ ID NO: 18, and at least one miRNA sequence with a guide strand sequence comprising SEQ ID NO: 7 and a scaffold sequence that shares at least 80% sequence identity to SEQ ID NO: 17.

In some embodiments, at least three miRNA sequences comprise at least one miRNA sequence with a guide strand sequence comprising SEQ ID NO: 5 and a scaffold sequence comprising SEQ ID NO: 18, and at least one miRNA sequence with a guide strand sequence comprising SEQ ID NO: 7 and a scaffold sequence comprising SEQ ID NO: 17.

In some embodiments, at least three miRNA sequences comprise one miRNA sequence with a guide strand sequence comprising SEQ ID NO: 2 and a scaffold sequence comprising SEQ ID NO: 16, one miRNA sequence with a guide strand sequence comprising SEQ ID NO: 5 and a scaffold sequence comprising SEQ ID NO: 18, and one miRNA sequence with a guide strand sequence comprising SEQ ID NO: 7 and a scaffold sequence comprising SEQ ID NO: 17.

In some embodiments, the present disclosure provides a recombinant adeno-associated virus (rAAV) vector comprising: a) a modified AAV genome comprising: (i) a promoter; (ii) at least one miRNA sequence; and b) a capsid; wherein at least one miRNA sequence comprises a guide strand sequence comprising SEQ ID NO: 2 and a miR-155 scaffold sequence, and wherein the miRNA sequence is operably linked to the promoter.

In some embodiments, the present disclosure provides a recombinant adeno-associated virus (rAAV) vector comprising: a) a modified AAV genome comprising: (i) a promoter; (ii) at least one miRNA sequence; and b) a capsid; wherein at least one miRNA sequence comprises a guide strand sequence comprising SEQ ID NO: 5 and a scaffold sequence, and wherein the miRNA sequence is operably linked to the promoter.

In some embodiments, a scaffold sequence comprises SEQ ID NO: 18.

In some embodiments, the present disclosure provides a recombinant adeno-associated virus (rAAV) vector comprising: a) a modified AAV genome comprising: (i) a promoter; (ii) at least one miRNA sequence; and b) a capsid; wherein at least one miRNA sequence comprises a guide strand sequence comprising SEQ ID NO: 7 and a scaffold sequence and wherein the miRNA sequence is operably linked to the promoter.

In some embodiments, a scaffold sequence comprises SEQ ID NO: 16, or SEQ ID NO: 17.

In some embodiments, a capsid is of serotype AAV1, AAV2, AAV3, AAV4, AAV5, AAV6, AAV7, AAV8, AAV9, AAV10, AAV11, AAV12, or variants or combinations thereof. In some embodiments, a capsid is or comprises AAV9. In some embodiments, a capsid is or comprises AAVrh.10.

In some embodiments, a modified AAV genome further comprises a nucleic acid sequence encoding a reporter protein. In some embodiments, a reporter protein is a luciferase protein, RFP, mCherry protein, GFP, or any variant and/or combination thereof. In some embodiments, a reporter protein is mCherry. In some embodiments, a reporter protein is GFP or a GFP variant.

In some embodiments, a promoter is CMV, EF1a, SV40, PGK, PGK1, Ubc, human beta-actin, beta-actin long (BActL), CAG, CBA, CBh, TRE, U6, H1, 7SK, ubiquitin C (UbiC), and any variant and/or combination thereof.

In some embodiment, a promoter is CAG, CMV, Synapsin, GFAP, or any combination thereof. In some embodiments, a promoter is a Pol II promoter. In some embodiments, a promoter is a Pol III promoter.

In some embodiments, a modified AAV genome further comprises a 3′ UTR element that enhances expression. In some embodiments, a 3′UTR element is a miRNA response element (MRE), AU-rich element (ARE), poly-A tail, Woodchuck Hepatitis Virus (WHV) Posttranscriptional Regulatory Element (WPRE), bovine growth hormone (bGH), human growth hormone (hGH), or any combination thereof. In some embodiments, a 3′UTR element is WPRE, bGH, hGH, p(A), or any combination thereof.

In some embodiments, an inhibitory nucleic acid provided herein does not comprise a WPRE. In some embodiments, an inhibitory nucleic acid comprises a polyadenylation (polyA) signal. In some embodiments, an inhibitory nucleic acid comprises a polyA signal selected from the group consisting of hGH polyA, bGH polyA, SV40 polyA, rb-Glob polyA, beta-Glob polyA, HSV TK polyA, and any combination thereof. In some embodiments, an inhibitory nucleic acid comprises a polyA signal having a nucleic acid sequence selected from any one of SEQ ID NOs. 45 or 58-64. In some embodiments, an inhibitory nucleic acid comprises a polyA signal having a nucleic acid sequence that is at least 70%, at least 75%, at least 80%, at least 85%, at least 90%, at least 95%, at least 98%, or at least 99% identical to a nucleic acid sequence selected from any one of SEQ ID NOs. 45 or 58-64. In some embodiments, a polyA signal blocks production of a minus strand transcribed from a 3′ITR.

In some embodiments, an AAV vector provides a guide strand to passenger strand ratio that is greater than 2.

In some embodiments, an AAV vector provides a guide strand production level of at least 0.01%, at least 0.1%, at least 1%, at least 2%, at least 3%, at least 4%, at least 5%, at least 10%, at least 15%, at least 20%, at least 25%, at least 30%, or at least 35%.

In some embodiments, an AAV vector provides a guide strand production level of at most 1%, at most 2%, at most 3%, at most 4%, at most 5%, at most 10%, at most 15%, at most 20%, at most 25%, at most 30%, or at most 35%.

In some embodiments, an AAV vector provides a guide strand potency that is greater than 50%.

In some embodiments, an AAV vector provides a guide strand accuracy of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, an AAV vector provides a guide strand accuracy of at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 99%.

In some embodiments, an AAV vector provides a guide strand accuracy that is greater than 80%.

In some embodiments, the present disclosure provides a pharmaceutical composition comprising an rAAV vector described in any one of the previous embodiments.

In some embodiments, the present disclosure provides a nucleic acid encoding an rAAV vector described in any one of the previous embodiments.

In some embodiments, the present disclosure provides a vector comprising a nucleic acid encoding an rAAV vector described in any one of the previous embodiments.

In some embodiments, the present disclosure provides a method of treating a subject with Amyotrophic Lateral Sclerosis (ALS), the method comprising a step of: administering a therapeutically effective amount of a composition that provides a recombinant adeno-associated virus (rAAV) vector that reduces SOD1 expression, wherein the rAAV vector is as described in any of one of the above embodiments.

In some embodiments, the present disclosure provides a method of treating a subject with Amyotrophic Lateral Sclerosis (ALS), the method comprising a step of: administering a therapeutically effective amount of a composition that provides a recombinant adeno-associated virus (rAAV) vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

In some embodiments, the present disclosure provides methods for simultaneously delivering two or more anti-SOD1 miRNAs to CNS tissue in a subject, the method comprising a step of: administering a therapeutically effective amount of a composition that provides a recombinant adeno-associated virus (rAAV) vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

In some embodiments, a therapeutically effective amount comprises an amount between a minimally effective amount and a maximally tolerable amount of a pharmaceutical composition. In some embodiments, a minimally effective amount comprises an amount of a pharmaceutical composition sufficient to reduce the level of SOD1 in a target tissue. In some embodiments, a minimally effective amount comprises an amount of a pharmaceutical composition sufficient to show a statistically significant improvement in one or more symptoms in a subject as compared to a subject not receiving treatment. In some embodiments, a maximally tolerable amount comprises an amount of a pharmaceutical composition at which toxicity or other effects of treatment results in one or more undesirable symptoms that are so severe that the benefit of treatment is outweighed.

In some embodiments, a composition is administered by intravenous administration, intrathecal administration, intracisternal administration, intramuscular administration, or combinations thereof.

In some embodiments, a capsid is of serotype AAV1, AAV2, AAV3, AAV4, AAV5, AAV6, AAV7, AAV8, AAV9, AAV10, AAV11, AAV12, or variants or combinations thereof.

In some embodiments, the present disclosure provides methods of inhibiting SOD1 expression in a cell, the method comprising a step of: administering a composition that provides a recombinant adeno-associated virus (rAAV) vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

In some embodiments, the present disclosure provides a recombinant adeno-associated virus (rAAV) vector comprising: a) a modified AAV genome comprising: (i) a promoter; and (ii) one or more miRNA sequences; and b) a capsid; wherein the one or more miRNA sequences comprise a guide strand sequence that targets SOD1, and a scaffold sequence and wherein the one or more miRNA sequences are operably linked to the promoter.

In some embodiments, one or more miRNA sequences comprise one or more guide strand sequences that share at least 80% sequence identity to a sequences selected from SEQ ID NOs: 1-12.

In some embodiments, the present disclosure provides methods of treating a subject with Amyotrophic Lateral Sclerosis (ALS), the method comprising co-administering: (i) a therapeutically effective amount of a composition that provides a rAAV particle provided herein; and (ii) one or more immunosuppressants. In some embodiments, an immunosuppressant is selected from the group consisting of Abrocitinib, Baricitinib, Cyclosporine, Dexamethoasone (Dex), intravenous immune globulin (IVIG), Mycophenolate Mofetil (MMF), Rituximab, Ruxolitinib, Sirolimus (Rapamycin), Tacrolimus (Tacro), Tofacitinib (Tofa), and Upadacitinib. In some embodiments, an immunosuppressant comprises or is an inhibitor of Janus Kinase (JAK). In some embodiments, an immunosuppressant comprises or is a steroid (e.g., Methylprednisolone or Prednisone). In some embodiments, an immunosuppressant is administered before administration of an rAAV particle provided herein. In some embodiments, an immunosuppressant is administered concurrently with an rAAV particle provided herein. In some embodiments, an immunosuppressant is administered following administration of an rAAV particle provided herein. In some embodiments, the period of time between administration of an rAAV particle provided herein and an immunosuppressant may be at least 1 day, at least 1 week, at least 2 weeks, at least 3 weeks, at least 4 weeks, at least 6 weeks, at least 8 weeks, or at least 12 weeks, at least 6 months, or at least 1 year or more. In some embodiments, an immunosuppressant is administered in multiple doses before and/or following administration of an rAAV particle provided herein. In some embodiments, an immunosuppressant is administered for a period of at least 1 day, at least 1 week, at least 2 weeks, at least 1 month, at least 2 months, at least 3 months, at least 6 months, or at least 1 year following administration of an rAAV particle provided herein. In some embodiments, an immunosuppressant is administered for a period of at least 1 day, at least 1 week, at least 2 weeks, at least 1 month, at least 2 months, at least 3 months, at least 6 months, or at least 1 year before administration of an rAAV particle provided herein. In some embodiments, an immunosuppressant may be administered before and after administration of an rAAV particle provided herein.

In some embodiments, the present disclosure provides a recombinant adeno-associated virus (rAAV) vector comprising a modified AAV genome comprising: (i) a promoter; and (ii) at least two or more different miRNA sequences, wherein each of the two or more miRNA sequences comprise a guide strand sequence that targets superoxide dismutase 1 (SOD1), and a scaffold sequence and wherein each of the two or more miRNA sequences are operably linked to the promoter.

In some embodiments, the present disclosure provides a recombinant adeno-associated virus (rAAV) vector comprising a modified AAV genome comprising: (i) a promoter; and (ii) at least one miRNA sequence, wherein at least one miRNA sequence comprises a guide strand sequence comprising SEQ ID NO: 2 and a miR-155 scaffold sequence, and wherein the miRNA sequence is operably linked to the promoter.

In some embodiments, the present disclosure provides a recombinant adeno-associated virus (rAAV) vector comprising a modified AAV genome comprising: (i) a promoter; and (ii) at least one miRNA sequence, wherein at least one miRNA sequence comprises a guide strand sequence comprising SEQ ID NO: 5 and a scaffold sequence, and wherein the miRNA sequence is operably linked to the promoter.

In some embodiments, the present disclosure provides a recombinant adeno-associated virus (rAAV) vector comprising a modified AAV genome comprising: (i) a promoter; and (ii) at least one miRNA sequence, wherein at least one miRNA sequence comprises a guide strand sequence comprising SEQ ID NO: 7 and a scaffold sequence and wherein the miRNA sequence is operably linked to the promoter.

In some embodiments, the present disclosure provides a method of treating a subject with Amyotrophic Lateral Sclerosis (ALS), the method comprising a step of: administering a therapeutically effective amount of a composition that provides a recombinant adeno-associated virus (rAAV) vector, wherein the rAAV vector comprises a modified AAV genome comprising: (i) a promoter; and (ii) two or more different miRNA sequences, wherein each of the two or more miRNA sequences comprise a guide strand that targets SOD1, and a scaffold sequence, and wherein each of the two or more miRNA sequences are operably linked to the promoter.

In some embodiments, the present disclosure provides a method for simultaneously delivering two or more anti-SOD1 miRNAs to CNS tissue in a subject, the method comprising a step of: administering a therapeutically effective amount of a composition that provides a recombinant adeno-associated virus (rAAV) vector, wherein the rAAV vector comprises a modified AAV genome comprising: (i) a promoter; and (ii) two or more different miRNA sequences, wherein each of the two or more miRNA sequences comprise a guide strand that targets SOD1, and a scaffold sequence, and wherein each of the two or more miRNA sequences are operably linked to the promoter.

In some embodiments, the present disclosure provides a method of inhibiting SOD1 expression in a cell, the method comprising a step of: administering a composition that provides a recombinant adeno-associated virus (rAAV) vector, wherein the rAAV vector comprises a modified AAV genome comprising: (i) a promoter; and (ii) two or more different miRNA sequences, wherein each of the two or more miRNA sequences comprise a guide strand that targets SOD1, and a scaffold sequence, and wherein each of the two or more miRNA sequences are operably linked to the promoter.

In some embodiments, the present disclosure provides recombinant adeno-associated virus (rAAV) vector comprising a modified AAV genome comprising: (i) a promoter; and (ii) one or more miRNA sequences, wherein the one or more miRNA sequences comprise a guide strand sequence that targets SOD1, and a scaffold sequence and wherein the one or more miRNA sequences are operably linked to the promoter.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adeno-associated virus (AAV): As used herein, the terms “Adeno-associated virus” and “AAV” refer to viral particles, in whole or in part, of family Parvoviridae and genus Dependoparvovirus. AAV is a small, replication-defective, non-enveloped virus. AAV may include, but is not limited to, AAV serotype 1, AAV serotype 2, AAV serotype 3 (including serotypes 3A and 3B), AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, AAV serotype rh10, AAV serotype rh74, AAV from the HSC 1-17 series, AAV from the CBr, CLv or CLg series, snake AAV, avian AAV, bovine AAV, canine AAV, equine AAV, ovine AAV, goat AAV, shrimp AAV, and any variant of any of the foregoing. AAV may also include engineered or chimeric versions of a wild-type AAV that include one or more insertions, deletions and/or substitutions within the Cap polypeptide(s) that affect one or more properties of the wild-type AAV serotype, including without limitation tropism and evasion of neutralizing antibodies (e.g., AAV-DJ, AAV-PHP.B, AAV-PHP.N, AAV.CAP-B1 to AAV.CAP-B25 and variants thereof). Wild-type AAV is replication deficient and requires co-infection of cells by a helper virus (e.g., adenovirus, herpes, or vaccinia virus) or supplementation of helper viral genes in order to replicate.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, vitreal, or any combination thereof. In some embodiments, administration maybe be intrathecal-lumbar puncture (LP). In some embodiments, administration may be intrathecal-intracisterna magna (ICM). In some embodiments, administration may be subpial injection, three-point injection of LP, ICM, and intracerebral ventricular (ICV), catheterized ICM, or any combination thereof. In some embodiments, a preferred method of administration will reduce or prevent an immune response from a subject receiving treatment.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, siRNAs, shRNAs, miRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Complementary: As used herein, the term “complementary” in the context of nucleic acid base-pairing refers to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

MicroRNA (miRNA): As used herein, the term “microRNA” or “miRNA” refers to a small, non-coding RNA molecule that can function in transcriptional and/or post-transcriptional regulation of target gene expression. The terms encompass a mature miRNA sequence or a precursor miRNA sequence, including a primary transcript (pri-miRNA) and a stem-loop precursor (pre-miRNA). The biogenesis of a naturally occurring miRNA initiates in the nucleus by RNA polymerase II transcription, generating a primary transcript (pri-miRNA). The primary transcript is cleaved by Drosha ribonuclease III enzyme to produce an approximately 70 nt stem-loop precursor miRNA (pre-miRNA). The pre-miRNA is then actively exported to the cytoplasm where it is cleaved by Dicer ribonuclease to form the mature miRNA, which includes an “antisense strand” or “guide strand” (that includes a region that is substantially complementary to a target sequence) and a “sense strand” or “passenger strand” (that includes a region that is substantially complementary to a region of the antisense strand). Those of ordinary skill in the art will appreciate that a guide strand may be perfectly complementary to a target region of a target RNA or may have less than perfect complementarity to a target region of a target RNA. The guide strand of this miRNA is incorporated into an RNA-induced silencing complex (RISC) that recognizes target mRNAs through base pairing with the miRNA, and commonly results in translational inhibition or destabilization of the target mRNA. As is understood in the field, for naturally occurring miRNAs, target mRNA recognition occurs through imperfect base pairing with the mRNA. In some embodiments, an miRNA is synthetic or engineered, and target mRNA recognition occurs through perfect base pairing with the mRNA. Typically, the target mRNA contains a sequence complementary to a “seed” sequence of the miRNA, which usually corresponds to nucleotides 2-8 of the miRNA. Information concerning miRNAs and associated pri-miRNA and pre-miRNA sequences is available in miRNA databases such as miRBase (Griffiths-Jones et al. 2008 Nucl Acids Res 36, (Database Issue: D154-D158) and the NCBI human genome database.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester scaffold. For example, in some embodiments, a nucleic acid is, comprises, or consists of one or more “peptide nucleic acids”, which are known in the art and have peptide bonds instead of phosphodiester bonds in the scaffold, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid can comprise or consist of one or more inhibitory nucleic acids (e.g., small RNA molecules). In some embodiments, an inhibitory nucleic acid comprises or consists of an RNA molecule (e.g., a small RNA molecule) that inhibits gene expression (e.g., via mRNA degradation) or inhibits translation (e.g., decreases the level of gene expression or translation of a transcript as compared to a relevant control). In some embodiments, an inhibitory nucleic acid comprises or consists of one or more siRNA, miRNA, shRNA, gRNA, or any combination thereof. In some embodiments, an inhibitory nucleic acid can be single stranded or double stranded.

Recombinant adeno-associated viral (rAAV) particle: A “recombinant adeno-associated viral (rAAV) particle”, or “rAAV particle,” as used herein, refers to an infectious, replication-defective viral particle comprising an AAV protein shell encapsulating at least one payload that is flanked on both sides by inverted terminal repeats (ITRs) in a vector. An rAAV particle can be produced in suitable host cells described herein (e.g., HEK293 cells, CHO-K cells, HeLa cells, or a variant thereof). For example, host cells are transfected with one or more vectors encoding: at least one payload flanked by an ITR on either side of the at least one payload, at least one Rep polypeptide, at least one Cap polypeptide, and at least one helper polypeptide, such that the host cells are capable of producing Rep, Cap and helper polypeptides necessary for packaging of rAAV particles. rAAV particles described herein may be used for subsequent gene delivery.

Subject: As used herein, the term “subject” or “patient” refers to any organism to which a provided composition is or may be administered, e.g., for experimental, diagnostic, prophylactic, cosmetic, and/or therapeutic purposes. In some embodiments, a subject is or comprises a cell or a tissue. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes a neurological disorder or condition. In some embodiments, such neurological disorder or condition is ALS.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, the term “vector” refers to an agent (e.g., an rAAV particle) capable of transporting a nucleic acid, wherein the agent comprises the nucleic acid. In some embodiments, a vector comprises or is an agent (e.g., a rAAV particle) capable of transporting a nucleic acid.

### BRIEF DESCRIPTION OF THE DRAWING

FIGS. 1A-1B show exemplary western blots showing miR-155-shRNA mediated knockdown of exogenous or endogenous human SOD1 in COS1 cells and HeLa cells.

FIG. 2 shows exemplary densitometry graphs showing remaining levels of human SOD1 protein normalized to GAPDH protein, after knockdown.

FIG. 3A-3C shows exemplary embedding rules for three miRNA scaffolds.

FIG. 4 shows an exemplary western blot showing miR-huSOD1 tested in three different miRNA scaffolds in AAV-transduced primary neuron culture expressing human SOD1 in vitro.

FIG. 5 shows an exemplary graph showing knockdown index of human SOD1 in primary neuronal cells treated with AAV-miR-huSOD1 vectors.

FIG. 6 shows exemplary RNA-seq results in human iPS-derived neuronal cells showing AAV-miR-huSOD1 vectors specifically target human SOD1 with minimal off target effects on the predicted hits based on sequence complementarity.

FIG. 7A-7B shows exemplary toxicity data based on serum neurofilament (pNFH) levels showing minimal toxicity in vivo for all miR-huSOD1 vectors except for miR-155-SOD1 #5.

FIG. 8 shows exemplary candidates of AAV9-miRNA-SOD1 assessed for their ability to block CMAP decline in SOD1-G93A mice, delivered in SOD1-G93A mice via ICV injection on P0, and monitored over time approximately every 4 weeks by CMAP recording of the tibialis muscle. Results represent the mean±SEM.

FIG. 9 shows exemplary mouse data showing increase in survival among mice treated with AAV-miR-SOD1 vectors.

FIG. 10 shows exemplary mice treated with four a-miR candidates showed lower levels of serum pNF-H compared with SOD1-G93A mice treated with control a-miR

FIG. 11 shows an exemplary AAV-miR-SOD1 duplex system.

FIG. 12 shows exemplary AAV-miR-SOD1 singlet and duplex systems.

FIG. 13 shows exemplary mouse data showing reduced serum pNFH levels in mice treated with AAV9-miRNA-SOD1 with weaker promoters, e.g., PGK, UbiC (Ubiquitin C), BActL (beta-actin long), or CBh, compared with mice treated with AAV9-miRNA-SOD1 with a CAG promoter.

FIG. 14 shows exemplary mouse data showing enhanced CMAP amplitude in mice treated with AAV9-miRNA-SOD1 with weaker promoters, e.g., PGK, UbiC (Ubiquitin C), BActL (beta-actin long), or CBh, compared with mice treated with ACSF.

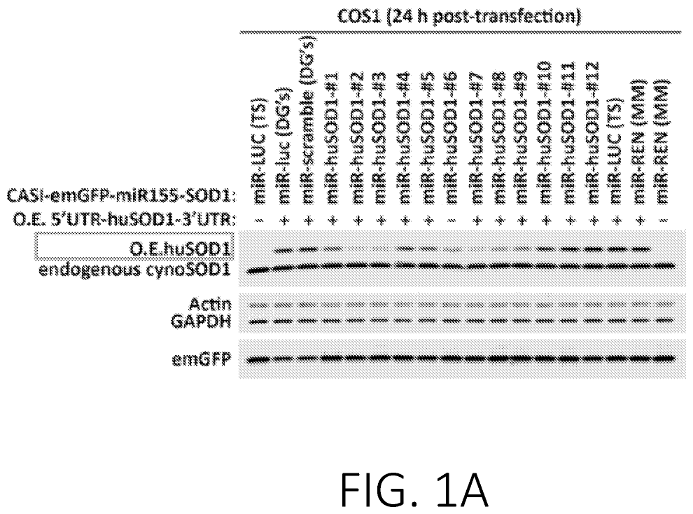

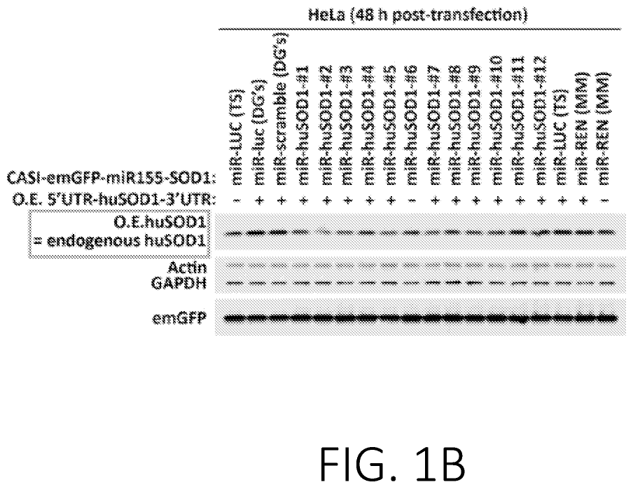

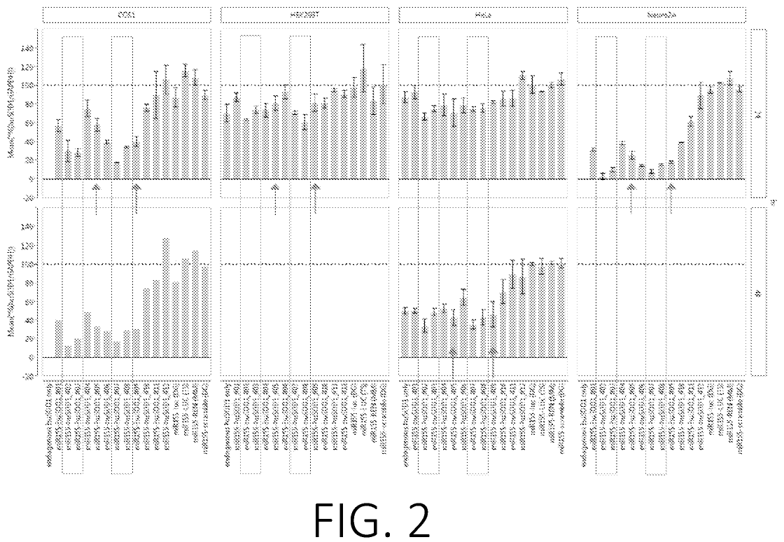

### DETAILED DESCRIPTION OF CERTAIN EMBODIMENTS

The present disclosure provides compositions and methods for treating amyotrophic lateral sclerosis (ALS). In some embodiments, the present disclosure provides inhibitory nucleic acids that inhibit the expression of genes that cause or are implicated in ALS pathogenesis. In some embodiments, the present disclosure provides recombinant adeno-associated virus (rAAV) vectors comprising inhibitory nucleic acids that inhibit the expression of genes that cause or are implicated in ALS pathogenesis. In some embodiments, the present disclosure provides compositions and methods for treating ALS that include rAAV vectors comprising one or more miRNAs that inhibit SOD1 expression. In some embodiments, the present disclosure provides compositions and methods for treating ALS that include rAAV vectors comprising at least two or more miRNAs that inhibit SOD1 expression. In some embodiments, miRNAs of the present disclosure are modified and/or engineered as compared to wild-type miRNAs. In some embodiments, inhibitory nucleic acids of the present disclosure target SOD1 mutants associated ALS disease pathogenesis.

The present disclosure further provides compositions and methods for treating ALS that exhibit reduced toxicity and/or immunoreactivity in a subject compared to compositions and methods known in the art. In some embodiments, methods that exhibit reduced toxicity and/or immunoreactivity in a subject comprise administration of rAAV vectors comprising inhibitory nucleic acids that inhibit expression of genes that cause or are implicated in ALS pathogenesis. Administration of compositions of the present disclosure may be by any method available to those skilled in the art. In some embodiments, the method of administration may be selected from the group of bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, vitreal administration, or any combination thereof. In some embodiments, administration may be intrathecal-lumbar puncture (LP). In some embodiments, administration may be intrathecal-intracisterna magna (ICM). In some embodiments, administration may be subpial injection, three-point injection of LP, ICM, and intracerebral ventricular (ICV), catheterized ICM, or any combination thereof. In some embodiments, administration may be conducted by any combination of administration methods described herein. In some preferred embodiments of the present disclosure, methods of treating ALS that exhibit reduced toxicity and/or immunoreactivity in a subject comprise administration of inhibitory nucleic acids (e.g., in the form of rAAV) by intrathecal injection.

### Amyotrophic Lateral Sclerosis

ALS is a fatal motor neuron disorder that is characterized by progressive loss of the upper and lower motor neurons (LMNs) at the spinal or bulbar level. ALS was first described in 1869 by French neurologist Jean-Martin Charcot. The disease became well known in the United States when baseball player Lou Gehrig was diagnosed with the disease in 1939. ALS is also known as Charcot disease in honor of the first person to describe the disease, Jean-Martin Charcot, and motor neuron disease (MND) as it is one of the five MNDs that affect motor neurons. There are four other known MNDs: Primary lateral sclerosis (PLS), progressive muscular atrophy (PMA), progressive bulbar palsy (PBP), and pseudobulbar palsy. The estimated prevalence of ALS in the United States from 2010 to 2011 was 12,187 persons (Mehta (2014)). Mutations within multiple genes (at least 10) are known to cause ALS (Renton (2014)), including mutations in the superoxide dismutase 1 (SOD1) gene (see below).

The present disclosure recognizes that ALS is categorized in two forms. The most common form is sporadic (90-95%) which has no obvious genetically inherited component. The remaining 5-10% of the cases are familial-type ALS (FALS) due to their associated genetic dominant inheritance factor. The first onset of symptoms is usually between the ages of 50 and 65. The most common symptoms that appear in both types of ALS are muscle weakness, twitching, and cramping, which eventually can lead to the impairment of muscles. In the most advanced stages, ALS patients will develop symptoms of dyspnea and dysphagia.

The present disclosure further recognizes that most common cause of ALS is a mutation of the gene encoding the antioxidant enzyme SOD1 (Dangoumau et al. (2014); De Vos et al. (2000); Jaiswal et al. (2014); Pasinelli et al. (2004); Vande Velde et al. (2008)). The frequency of SOD1 mutations is estimated to be 10% to 20% of familial ALS and 2% to 4% of apparently sporadic ALS, though regional variation likely exists (Akimoto (2011); Byrne (2011); Chiò (2012); Chiò (2008)). Mutant SOD1 has a structural instability that causes a misfold in the mutated enzyme, which can lead to aggregation in the motor neurons within the central nervous system (CNS) (Forsberg et al. (2011)). The present disclosure encompasses the recognition that several hypotheses have been proposed in regards to the mechanism underlying the mode of action of mutant SOD and the subsequent neurodegeneration seen in ALS. The most important proposed hypotheses for the pathogenesis of ALS includes glutamate excitotoxicity structural and functional abnormalities of mitochondria, impaired axonal structure or transport defects, and free radical-mediated oxidative stress (De Vos et al. (2000); Donnelly et al. (2013); Forsberg et al. (2011); Jaiswal et al. (2014); Magrane et al. (2009); Mitsumoto et al. (2014); Shi et al. (2010); Wang et al. (2015); Zhu et al. (2011)). Even though these mechanisms play a critical role in neurodegeneration, they all are considered as secondary events in the causes behind ALS onset (Vucic et al. (2007)).

Eukaryotic SOD1 is a 32-kDa homodimeric metalloenzyme, found predominantly in the cytosol, but also in the mitochondrial intermembrane space, nucleus, and peroxisomes. Each of the two subunits of SOD1 forms an eight-stranded Greek key beta-barrel and contains an active site that binds a catalytic copper ion (binding residues: His46, His48, His63 and His120) and a structural zinc ion (binding residues: His63, His71, His80 and Asp83). Its functional role is that of catalyzing the dismutation of superoxide radical to dioxygen and hydrogen peroxide (Fridovich et al. (1978); Bertini et al. (1998)). The mature, correctly folded and enzymatically active form of SOD1 is obtained in vivo through several post-translational modifications: acquisition of zinc and copper ions, disulfide bond formation, and dimerization (Valentine et al. (2005); Culotta et al. (2006); Arnesano et al. (2004)). The present disclosure encompasses the recognition that at least about 100 single point mutations of SOD1 are reported (http://alsod.iop. kcl.ac.uk/Als/index.aspx) to be related to the familial form of ALS. The present disclosure further recognizes that over 180 different mutations overall, including single point mutations, deletions, insertions, and truncation mutations, have been identified throughout the five exons of the SOD1 gene. In some embodiments, inhibitory nucleic acids as described herein may be designed to inhibit expression of any of the aforementioned SOD1 mutants that are associated with ALS. In some particular embodiments, inhibitory nucleic acids as described herein are designed to inhibit expression of SOD1 genes comprising point mutations F20C, E21G, G10V, C6S, K3E, L106V, L144F, D90A, A4V, G93A, or any combination thereof.

### Inhibitory Nucleic Acids

In some embodiments, the present disclosure provides inhibitory nucleic acids that inhibit the expression of genes that cause or are implicated in ALS pathogenesis. In some embodiments, the present disclosure provides inhibitory nucleic acids that target nucleic acids produced from genes that cause or are implicated in ALS pathogenesis. In some embodiments of the present disclosure, inhibitory nucleic acids comprise RNA molecules that inhibit gene expression by hybridizing to target nucleic acids produced by a gene of interest, e.g., RNA interference, CRISPR, etc. In some embodiments, inhibitory nucleic acids of the present disclosure include, but are not limited to, siRNA, shRNA, miRNA, gRNA, or any combination thereof. In some preferred embodiments, inhibitory nucleic acids of the present disclosure comprise one or more miRNAs. In some preferred embodiments, inhibitory nucleic acids of the present disclosure comprise two or more miRNAs. In some embodiments, miRNAs of the present disclosure comprise a guide strand sequence that targets a target nucleic acid of interest. In some embodiments, inhibitory nucleic acids are single stranded or double stranded. In some embodiments, inhibitory nucleic acids of the present disclosure are flanked by and/or operably linked to structural and/or regulatory nucleic acid sequences, for example those described herein. In some preferred embodiments, the present disclosure provides inhibitory nucleic acids that inhibit SOD1 expression. In some embodiments, the present disclosure provides inhibitory nucleic acids comprising one or more miRNAs that inhibit SOD1 expression. In some embodiments, the present disclosure provides inhibitory nucleic acids comprising at least two or more miRNAs that inhibit SOD1 expression. In some embodiments, the present disclosure provides inhibitory nucleic acids comprising at least two or more different miRNAs that inhibit SOD1 expression. In some embodiments, mutant variants of SOD1, such as those common in ALS and described herein, are preferentially targeted by inhibitory nucleic acids of the present disclosure.

In some embodiments, the present disclosure provides inhibitory nucleic acids between 19 and 30 bases in length. In some embodiments, provided inhibitory nucleic acids are between 15 and 20, between 20 and 25, or between 25 and 30 bases in length. In some embodiments, the present disclosure provides inhibitory nucleic acids that are at least 30, at least 29, at least 28, at least 27, at least 26, at least 25, at least 24, at least 23, at least 22, at least 21, at least 20, at least 19, at least 18, at least 17, at least 16, or at least 15 bases in length. In some embodiments, the present disclosure provides inhibitory nucleic acids that are at most 30, at most 29, at most 28, at most 27, at most 26, at most 25, at most 24, at most 23, at most 22, at most 21, at most 20, at most 19, at most 18, at most 17, at most 16, or at most 15 bases in length. In some embodiments, inhibitory nucleic acids can be single stranded or double stranded.

In some embodiments, inhibitory nucleic acids of the present disclosure comprise or consist of one or more inhibitory nucleic acid sequences that are complementary to one or more target nucleic acids (e.g., guide sequences). In some embodiments of the present disclosure, inhibitory nucleic acids comprise or consist of one or more inhibitory nucleic acid sequences that are complementary to at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, or at least 50% of bases in a target nucleic acid sequence. In some embodiments of the present disclosure, inhibitory nucleic acids comprise or consist of one or more inhibitory nucleic acid sequences that are complementary to at most 99%, at most 98%, at most 97%, at most 96%, at most 95%, at most 94%, at most 93%, at most 92%, at most 91%, at most 90%, at most 89%, at most 88%, at most 87%, at most 86%, at most 85%, at most 84%, at most 83%, at most 82%, at most 81%, at most 80%, at most 79%, at most 78%, at most 77%, at most 76%, at most 75%, at most 74%, at most 73%, at most 72%, at most 71%, at most 70%, at most 69%, at most 68%, at most 67%, at most 66%, at most 65%, at most 64%, at most 63%, at most 62%, at most 61%, at most 60%, at most 59%, at most 58%, at most 57%, at most 56%, at most 55%, at most 54%, at most 53%, at most 52%, at most 51%, or at most 50% of bases in a target nucleic acid sequence.

In some embodiments, the present disclosure provides inhibitory nucleic acids that comprise or consist of one or more inhibitory nucleic acid sequences that are complementary to at least 35, at least 34, at least 33, at least 32, at least 31, at least 30, at least 29, at least 28, at least 27, at least 26, at least 25, at least 24, at least 23, at least 22, at least 21, at least 20, at least 19, at least 18, at least 17, at least 16, at least 15, at least 14, at least 13, at least 12, at least 11, at least 10, at least 9, at least 8, at least 7, at least 6, or at least 5 bases in a target nucleic acid sequence. In some embodiments, the present disclosure provides inhibitory nucleic acids that comprise or consist of one or more inhibitory nucleic acid sequences that are complementary to at most 35, at most 34, at most 33, at most 32, at most 31, at most 30, at most 29, at most 28, at most 27, at most 26, at most 25, at most 24, at most 23, at most 22, at most 21, at most 20, at most 19, at most 18, at most 17, at most 16, at most 15, at most 14, at most 13, at most 12, at most 11, at most 10, at most 9, at most 8, at most 7, at most 6, or at most 5 bases in a target nucleic acid sequence.

In some embodiments, inhibitory nucleic acids of the present disclosure can contain contiguous and/or non-contiguous base mismatches within regions that are substantially complementarity to a target nucleic acid. In some embodiments of the present disclosure, inhibitory nucleic acids comprise one or more base mismatches within regions that are substantially complementary to a target nucleic acid. In some embodiments, inhibitory nucleic acids comprise at least 5, at least 4, at least 3, or at least 2 base mismatches that are contiguous within regions that are substantially complementarity to a target nucleic acid. In some embodiments, inhibitory nucleic acids comprise at most 5, at most 4, at most 3, or at most 2 base mismatches that are contiguous within regions that are substantially complementarity to a target nucleic acid. In some embodiments, the present disclosure provides inhibitory nucleic acids that comprise at least 10, at least 9, at least 8, at least 7, at least 6, at least 5, at least 4, at least 3, or at least 2 base mismatches that are non-contiguous within regions that are substantially complementarity to a target nucleic acid sequence. In some embodiments, the present disclosure provides inhibitory nucleic acids that comprise at most 10, at most 9, at most 8, at most 7, at most 6, at most 5, at most 4, at most 3, or at most 2 base mismatches that are non-contiguous within regions that are substantially complementarity to a target nucleic acid sequence

In some embodiments, the present disclosure provides inhibitory nucleic acids that comprise or consist of inhibitory nucleic acid sequences that are substantially complementary to a target nucleic acid sequence. In some embodiments, a target nucleic acid sequence is a SOD1 nucleic acid sequence. In some embodiments, inhibitory nucleic acid sequences comprise or consist of miRNA, siRNA, shRNA, gRNA, or any combination thereof. In some preferred embodiments, inhibitory nucleic acid sequences of the present disclosure comprise or consist of one or more miRNA. In some embodiments, miRNA of the present disclosure comprise guide strand sequences that are substantially complementary to one or more target nucleic acid sequences. In some embodiments of the present disclosure, a target nucleic acid sequence comprises a wild-type SOD1 nucleic acid sequence, or mutant or variant SOD1 nucleic acid sequence. In some embodiments, targeted SOD1 nucleic acid sequences include SOD1 mRNA sequences. In some embodiments, targeted SOD1 mRNA sequences comprise sequences from human SOD1 mRNA. In some embodiments, targeted SOD1 mRNA sequences comprise sequences from human SOD1 mRNA as set forth in SEQ ID NO: 46 (NM_00454.4). In some embodiments, inhibitory nucleic acid sequences of the present disclosure are at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, or at least 40% complementary to a wild type SOD1 nucleic acid sequence, or a mutant or variant SOD1 nucleic acid sequence, that is known in the art, including those that are described herein. In some embodiments, inhibitory nucleic acid sequences of the present disclosure are at most 99%, at most 98%, at most 97%, at most 96%, at most 95%, at most 94%, at most 93%, at most 92%, at most 91%, at most 90%, at most 89%, at most 88%, at most 87%, at most 86%, at most 85%, at most 84%, at most 83%, at most 82%, at most 81%, at most 80%, at most 79%, at most 78%, at most 77%, at most 76%, at most 75%, at most 74%, at most 73%, at most 72%, at most 71%, at most 70%, at most 69%, at most 68%, at most 67%, at most 66%, at most 65%, at most 64%, at most 63%, at most 62%, at most 61%, at most 60%, at most 59%, at most 58%, at most 57%, at most 56%, at most 55%, at most 54%, at most 53%, at most 52%, at most 51%, at most 50%, at most 49%, at most 48%, at most 47%, at most 46%, at most 45%, at most 44%, at most 43%, at most 42%, at most 41%, or at most 40% complementary to a wild type SOD1 nucleic acid sequence, or a mutant or variant SOD1 nucleic acid sequence, that is known in the art, including those that are described herein. In some embodiments, inhibitory nucleic acid sequences of the present disclosure comprise or consist of one or more of SEQ ID NOs: 1-12. In some embodiments, inhibitory nucleic acid sequences of the present disclosure comprise or consist of two or more of SEQ ID NOs: 1-12. In some embodiments, inhibitory nucleic acid sequences of the present disclosure comprise or consist of two of SEQ ID NOs: 1-12.

In some embodiments, inhibitory nucleic acid sequences of the present disclosure may be designed to have cross-reactivity with a non-target nucleic acid sequence. In some embodiments cross-reactivity means an inhibitory nucleic acid has competing affinity between a target nucleic acid sequence and a non-target nucleic acid sequence. In some embodiments, a target nucleic acid sequence and a non-target nucleic acid sequence are from different species. In some particular embodiments, a target nucleic acid sequence is a human target nucleic acid sequence and a non-target nucleic acid sequence is a non-human nucleic acid sequence. In some embodiments, a non-target nucleic acid sequence is a Mus musculus, Macaca fascicularis, Callithrix iachus, or Macaca mulatta nucleic acid sequence. In some embodiments, a target nucleic acid sequence and a non-target nucleic acid sequence are SOD1 nucleic acid sequences from different species. In some embodiments, a targeted nucleic acid sequence and a non-targeted nucleic acid sequence comprise sequences from SOD1 mRNA. In some embodiments, SOD1 mRNA sequences comprise sequences from SOD1 mRNA as set forth in SEQ ID NOs: 45-50.

In some embodiments, inhibitory nucleic acids of the present disclosure, as described herein, inhibit expression of genes that cause or are implicated in neurological diseases or disorders (e.g., ALS). In some embodiments of the present disclosure, inhibitory nucleic acids inhibit gene expression by hybridizing to target nucleic acids produced by a gene of interest, e.g., by RNA interference, CRISPR, etc. In some embodiments, a cell or tissue treated with inhibitory nucleic acids of the present disclosure exhibits a reduction in expression of a target nucleic acid of least 10%, at least 20%, at least 30%, at least 40%, at least 50%, at least 60% at least 70%, at least 80%, or at least 90% compared to expression of a target nucleic acid in a cell or tissue not treated with inhibitory nucleic acids of the present disclosure. In some embodiments, a cell or tissue treated with inhibitory nucleic acids of the present disclosure exhibits a reduction in expression of a target nucleic acid of most 20%, at most 30%, at most 40%, at most 50%, at most 60% at most 70%, at most 80%, or at most 90% compared to expression of a target nucleic acid in a cell or tissue not treated with inhibitory nucleic acids of the present disclosure.

In some embodiments, the present disclosure recognizes that guide strand to passenger strand ratio provided by an inhibitory nucleic acid (e.g., miRNA) plays a role in effective targeting of a target nucleic acid. In some embodiments, inhibitory nucleic acids provide a guide strand to passenger strand ratio of at least 2 or at least 3 when administered to a subject. In some embodiments, inhibitory nucleic acids provide a guide strand to passenger strand ratio greater than 2. In some embodiments, the present disclosure recognizes that guide strand production level plays a role in effective targeting of a target nucleic acid. Guide strand production level may be defined as percent of the sequencing reads that match a guide strand of a miRNA (e.g., artificial miRNA) relative to total number of sequencing reads matching all mature endogenous miRNAs in a sample. This is a proxy for the number of a-miR guide strand molecules relative to the number of endogenous miRNA molecules, expressed as a percentage. In some embodiments, inhibitory nucleic acids provide a guide strand production level of at least 0.01%, at least 0.1%, at least 1%, at least 2%, at least 3%, at least 4%, at least 5%, at least 10%, at least 15%, at least 20%, at least 25%, at least 30%, or at least 35%. In some embodiments, inhibitory nucleic acids provide a guide strand production level of at most 1%, at most 2%, at most 3%, at most 4%, at most 5%, at most 10%, at most 15%, at most 20%, at most 25%, at most 30%, or at most 35%. In some embodiments, the present disclosure recognizes that guide strand potency, which may be defined as the percent decrease of a target gene (e.g., human SOD1) expression levels, of certain inhibitory nucleic acids can be used to select an inhibitory nucleic acid to effectively target a target nucleic acid. In some embodiment, guide strand accuracy of certain inhibitory nucleic acids is recognized by the present disclosure to play a role in effective targeting of a target nucleic acid. Guide strand accuracy may be defined as the fraction of a-miR guide strands that match a designed sequence with maximum one nucleotide mismatch, and further, have the exact length of the designed sequence or are longer. In some embodiments, inhibitory nucleic acids of the present disclosure provide guide strand accuracy of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, inhibitory nucleic acids of the present disclosure provide guide strand accuracy of at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 99%. In some embodiments, inhibitory nucleic acids of the present disclosure provide guide strand accuracy greater than 80%.

In some embodiments, the present disclosure provides inhibitory nucleic acids that comprise or consist of one or more miRNAs that inhibit the expression of genes that cause or are implicated in ALS pathogenesis. In some embodiments, miRNAs of the present disclosure comprise scaffold sequences of wild type miRNAs. In some embodiment the present disclosure, wild type miRNA scaffold sequences include, but are not limited to, miR-155, miR-30a, mIR-122, miR-150, miR-21, miR-20a, miR-16-1, and combinations thereof. It is contemplated that any wild type miRNA scaffold known by those skilled in the art to facilitate inhibition of a target nucleic acid can be utilized in accordance with the present disclosure. In some embodiments, miRNAs of the present disclosure comprise modified and/or engineered miRNA scaffolds. Non-limiting examples of modified and engineered miRNA scaffolds include miR-E, miR-3G, miR-16-2, ultramiR, engineered variants of miR-155, or any combination thereof. In some embodiments of the present disclosure, miRNA scaffolds discussed herein comprise one or more of SEQ ID NOs: 1-12.

In some embodiments, the present disclosure provides inhibitory nucleic acids that comprise or consist of two or more miRNAs that inhibit the expression of genes that cause or are implicated in ALS pathogenesis. In some embodiments, two or more miRNAs of the present disclosure are directly linked, e.g., from 3′ of one miRNA to 5′ of a second miRNA. In some embodiments, two or more miRNAs of the present disclosure are linked by a spacer. In some embodiments, an exemplary spacer is or comprises nucleotide sequence GC. In some embodiments, an exemplary spacer is or comprises nucleotide sequence GGTACC.

In some embodiments, the present disclosure provides inhibitory nucleic acids that comprise or consist of multiple (e.g., at least two) miRNAs that inhibit expression of genes that cause or are implicated in ALS pathogenesis. In some embodiments, two miRNAs of an inhibitory nucleic acid provided herein are different miRNAs (e.g., a hetero-duplex design). In some embodiments, inhibitory nucleic acids having a hetero-duplex design provide enhanced efficacy in patients with one or more point mutations in one or more miR-targeted loci. In some embodiments, inhibitory nucleic acids having a hetero-duplex design provide broad efficacy in different cell types, species (e.g., primates), and/or disease states in which one a-miR backbone is not efficiently processed.

In some embodiments, inhibitory nucleic acids of the present disclosure are modified to include one or more chemically modified nucleotides to obtain one or more desirable qualities (e.g., enhanced silencing of a target gene, enhanced stability, or combinations thereof). In some embodiments, chemically modified nucleotides of the present disclosure include, but are not limited to, 2′-deoxy nucleotides, 2′-OMe nucleotides, thioate linked nucleotides, 2′-fluorouridine, 2′-fluorocytidine, N3-methyluridine, 5-bromouridine, 5-iodouridine, 2,6-diaminopurine, and combinations thereof.

Recombinant Adeno-Associated Virus (rAAV)

In some embodiments, the present disclosure provides recombinant AAV vectors comprising inhibitory nucleic acids that inhibit the expression of genes that cause or are implicated in ALS pathogenesis. In some embodiments of the present disclosure, inhibitory nucleic acids comprise RNA molecules that inhibit gene expression by hybridizing to target nucleic acids produced by a gene of interest, e.g., RNA interference, CRISPR, etc. In some embodiments, inhibitory nucleic acids of the present disclosure include, but are not limited to, siRNA, shRNA, miRNA, gRNA, or combinations thereof. In some preferred embodiments, inhibitory nucleic acids of the present disclosure comprise miRNAs. In some embodiments, inhibitory nucleic acids are single stranded or double stranded. In some embodiments, inhibitory nucleic acids of the present disclosure are flanked by and/or operably linked to structural and/or regulatory nucleic acid sequences, for example those described herein. In some preferred embodiments, the present disclosure provides recombinant AAV vectors comprising inhibitory nucleic acids that inhibit SOD1 expression. In some embodiments, the present disclosure provides recombinant AAV vectors comprising inhibitory nucleic acids comprising one or more miRNAs that inhibit SOD1 expression. In some embodiments, the present disclosure provides recombinant AAV vectors comprising inhibitory nucleic acids comprising at least two or more miRNAs that inhibit SOD1 expression. In some embodiments, the present disclosure provides recombinant AAV vectors comprising inhibitory nucleic acids comprising at least two or more different miRNAs that inhibit SOD1 expression.

Structure

AAV is a small, non-enveloped virus that packages a single-stranded linear DNA genome, approximately 5 kb long. A member of the family Parvoviridae, AAV was discovered in 1965 as a contaminant of Ad isolates. AAV has not been associated with any human or animal disease, even though most humans (>70%) are seropositive for one or more serotypes (Calcedo et al. (2011); Calcedo et al. (2009)). Both positive and negative DNA strands are packaged equally well, and infection can be initiated with particles containing either strand. The virus has a T=1 icosahedral capsid, 25 nm in diameter, that is extraordinarily stable. It resists brief exposure to heat, acidic pH, and proteases. The viral genome consists of three open reading frames (ORFs) that code for eight proteins (Rep78, Rep68, Rep52, Rep40, VP1, VP2, VP3, and AAP) expressed from three promoters (p5, p19, and p40). The mature capsid consists of the amino acid sequence of only one ORF (cap) and the packaged DNA. Thus, recombinant AAV (rAAV) vectors present a very small target for the host immune system.

The present disclosure recognizes that the coding regions of AAV are flanked by inverted terminal repeats (ITRs) that are 145 bases long and have a complex T-shaped structure. These repeats are the origins for DNA replication and serve as the primary packaging signal (McLaughlin et al. (1988); Hauswirth et al. (1977)). The present disclosure further recognizes that ITRs are the only cis-active sequences required for making rAAV vectors and the only AAV-encoded sequences present in AAV vectors (McLaughlin et al. (1988); Samulski et al. (1989)). Although the AAV ITRs have enhancer activity in the presence of Rep protein, they have minimal promoter or enhancer activity in the absence of Rep protein. Thus, transgenes cloned into an AAV vector must be engineered with appropriate enhancer, promoter, poly(A), and splice signals to ensure correct gene expression.

In some embodiments, inhibitory nucleic acids of the present disclosure are flanked by and/or operably linked to structural and/or regulatory nucleic acid sequences including ITR sequences, promoters, enhancers, 5′ regulatory elements, 3′ regulatory elements, and any combinations thereof. In some embodiments, structural and/or regulatory nucleic acid sequences described herein are operably linked to the inhibitory nucleic acids of the present disclosure in order to facilitate or aid in the transcription of said inhibitory nucleic acids.

In some embodiments, ITR sequences of the present disclosure can include ITR sequences from any AAV serotype. In some embodiments, ITR sequences of the present disclosure can include ITR sequences from the group consisting of AAV1, AAV2, AAV3, AAV4, AAV5, AAV6, AAV7, AAV8, AAV9, AAV10, AAV11, AAV12, or any combinations thereof. In some embodiments, ITR sequences of the present disclosure may comprise engineered or modified ITR sequences using methods known in the art.

The present disclosure provides, among other things, inhibitory nucleic acids that can be operably linked to any promoter that facilitates transcription of the inhibitory nucleic acid. In some embodiments, inhibitory nucleic acids of the present disclosure are operably linked to a constitutive or inducible promoter. In some embodiments, inhibitory nucleic acids of the present disclosure are operably linked to promoters selected from the group consisting of CMV, EF1a, SV40, PGK, PGK1, Ubc, human beta-actin, beta-actin long (BActL), CAG, CBA, CBh, TRE, U6, H1, 7SK, ubiquitin C (UbiC) and any combinations thereof. In some embodiments, inhibitory nucleic acids of the present disclosure are operably linked to promoters selected from the group consisting of PGK, beta-actin long (BActL), CBh, ubiquitin C (UbiC) and any combinations thereof. In some embodiments, inhibitory nucleic acids of the present disclosure are operably linked to promoters that are chosen for their reduced transcriptional efficiency relative to CAG. In some embodiments, inhibitory nucleic acids of the present disclosure are operably linked to a modified or engineered promoter. In some embodiments inhibitory nucleic acids of the present disclosure are operably linked to tissue or cell specific promoters to enable targeting of a subset of tissues or cells that are particularly affected in a disease or disorder of interest (e.g., ALS). In some embodiments, inhibitory nucleic acids of the present disclosure are operably linked to one or more (e.g., one or more, two or more, three or more, four or more, etc.) promoters as described herein.

In some embodiments of the present disclosure, inhibitory nucleic acids may be operably linked to 5′ regulatory elements and/or 3′ regulatory elements. In some embodiments, of the present disclosure, inhibitory nucleic acids may also comprise intronic sequences. In some embodiments, inhibitory nucleic acids may comprise 5′ untranslated and 3′ untranslated regions as required. In some embodiments, the present disclosure provides inhibitory nucleic acids comprising sequences involved with transcription such as TATA box, capping sequences, CAAT sequences, enhancer elements, IRES, and combinations thereof. In some embodiments of the present disclosure, 3′ regulatory elements may be selected from the group consisting of poly-A tails, AU-rich elements, and combinations thereof. In some embodiments, sequences involved in transcription include Woodchuck Hepatitis Virus (WHV) Posttranscriptional Regulatory Element (WPRE) and P2A. In some embodiments, an inhibitory nucleic acid provided herein does not comprise a WPRE. In some embodiments, an inhibitory nucleic acid comprises a polyadenylation (polyA) signal. In some embodiments, an inhibitory nucleic acid comprises a polyA signal selected from the group consisting of hGH polyA, bGH polyA, SV40 polyA, rb-Glob polyA, beta-Glob polyA, HSV TK polyA, and any combination thereof. In some embodiments, an inhibitory nucleic acid comprises a polyA signal having a nucleic acid sequence selected from any one of SEQ ID NOs. 45 or 58-64. In some embodiments, an inhibitory nucleic acid comprises a polyA signal having a nucleic acid sequence that is at least 70%, at least 75%, at least 80%, at least 85%, at least 90%, at least 95%, at least 98%, at least 99% identical to a nucleic acid sequence selected from any one of SEQ ID NOs. 45 or 58-64. In some embodiments, a polyA signal blocks production of a minus strand transcribed from a 3′ITR.

In some additional embodiments of the present disclosure, recombinant AAV may comprise reporter protein sequences that are operably linked to a promoter. In some embodiments, reporter protein sequences may be green fluorescent protein (GFP) or any variants thereof. In some embodiments, report protein sequences may be a luciferase protein or any variants thereof.

rAAV Inhibitory Nucleic Acids

The present disclosure provides, among other things, recombinant AAV vectors comprising a modified AAV genome comprising inhibitory nucleic acids that inhibit the expression of genes that cause or are implicated in ALS pathogenesis. In some embodiments, inhibitory nucleic acids of the present disclosure comprise one or more miRNAs. In some embodiments, inhibitory nucleic acids of the present disclosure comprise at least two or more miRNAs. In many embodiments, miRNAs of the present disclosure comprise guide strand sequences that target a target nucleic acid of interest. In some embodiments, miRNAs of the present disclosure comprise a guide strand sequence that is substantially complementary to a target nucleic acid of interest. In some embodiments, miRNAs of the present disclosure comprise a guide strand sequence that targets a target nucleic acid of interest. In some embodiments, miRNAs of the present disclosure comprise one or more guide strand sequences that comprise or consist of one or more sequences as set forth in SEQ ID NOs: 1-12. In some embodiments, miRNAs of the present disclosure comprise guide strand sequences that comprise or consist of SEQ ID NO: 5 and SEQ ID NO: 7. In some embodiments, miRNAs of the present disclosure comprise scaffold sequences of wild type and/or modified and engineered miRNAs as described herein.

rAAV Capsids

The present disclosure encompasses the recognition that more than 110 distinct primate AAV capsid sequences have been isolated. Each of those AAV capsids that have unique serological profiles has been named as a particular AAV serotype. The present disclosure further appreciates that at least 12 primate serotypes (AAV1-12) have been described. In some embodiments of the present disclosure, a capsid from any serotype can be used. In some embodiments, a modified or engineered capsid including, but not limited to those described herein, can be used in accordance with the present disclosure.

The present disclosure recognizes that numerous studies have evaluated and compared serotypes with regard to their transduction efficiency in tissues in vivo. For example, in striated muscle, studies achieved high transduction efficiency with AAV1, AAV6, and AAV7. Similarly, AAV8 and AAV9 have been found to transduce striated muscle with efficiencies at least as high. rAAV8 and rAAV9 are considered to have the highest level of hepatocyte transduction. In the pulmonary system, rAAV6 and rAAV9 transduce much of the entire airway epithelium, while rAAV5 transduction is limited to lung alveolar cells. With respect to transduction of the central nervous system, rAAV serotypes 1, 4, 5, 7, and 8 have been found to be efficient transducers of neurons in various regions of the brain. rAAV1 and rAAV5 have also been reported to transduce ependymal and glial cells. In the eye, rAAV serotypes 1, 4, 5, 7, 8, and 9 efficiently transduce retinal pigmented epithelium, while rAAV5, rAAV7, and rAAV8 transduce photoreceptors as well. rAAV1, rAAV8, and rAAV9 have shown the highest reported transduction in pancreas tissue, primarily in acinar cells. The kidney appears to be a relatively difficult organ to transduce, although proximal tubule cells have been transduced by rAAV2 at low levels, as have glomeruli by rAAV9. Additionally, rAAV1 has been shown to transduce adipose tissue, albeit with the aid of a nonionic surfactant.

The present disclosure additionally encompasses the recognition that it may be advantageous to modify wild type AAV capsids, or engineer AAV capsids, to achieve designer tissue tropism and/or immune system evasion. One method of achieving this is to produce vector in the presence of cap genes for multiple serotypes. Depending on the ratio of capsid proteins from each serotype, the resulting “mosaic” virions can exhibit a combined tropism for cell type or, in some cases, can acquire tropism not exhibited by either serotype individually. Some studies have involved attaching exogenous molecules to the capsid. One example utilizes a bi-specific antibody obtained by fusing Fc regions of two different antibodies: an anti-capsid antibody and an anti-cell marker antibody, thereby conferring rAAV2 tropism to transduction-resistant megakaryocyte cell lines. Another example adopted the approach of biotinylating the capsid and subsequently binding it to a streptavidin conjugate carrying epidermal growth factor or fibroblast growth factor. This approach was shown to produce at least a tenfold increase in the transduction of cells that highly express the epidermal growth factor or fibroblast growth factor receptor, respectively.

The present disclosure also appreciates that as an alternative to attaching molecules to the capsid surface, it may be advantageous to engineer a modification directly into the cap gene. As one non-limiting example, green fluorescent protein (GFP) (238 amino acids) can be inserted into AAV2 VP1 and VP2. Although the transduction efficiencies of the VP1-GFP and VP2-GFP vectors were 3 and 5 orders of magnitude lower, respectively, than the efficiency of wild-type capsid, the transduction in HeLa cells did occur, suggesting a tolerance for inserted sequences in capsid proteins. As another non-limiting example, for modifying cap genes for tissue targeting, a number of researchers have inserted peptide sequences on the basis of known ligand-receptor interactions, or have selected for peptides in phage-display libraries. Another strategy has been to insert random sequences of amino acids, followed by in vitro selection of the best performing capsids. Instead of introducing target-specific peptides, some experiments modified the capsids generically, pending subsequent modification toward targets of choice. For example, a binding site for the Fc portion of antibodies was inserted into the capsid, followed by binding of different antibodies specific for receptors of various cell lines. Another such modification is to insert a biotin-binding site into the capsid, thereby facilitating metabolic biotinylation and allowing flexible targeting with any avidin-conjugated ligands. Some experiments have taken advantage of peptide insertion as well as mosaic capsids with a virion containing both wild-type capsid proteins and engineered capsid proteins, or a virion containing a combination of multiple different modified capsid proteins. Other techniques are under investigation with a view to evading the immune system, and these include coating capsids with polymer.

rAAV Production

Methods of producing and isolating rAAV with a desired inhibitory nucleic acid, or transgene, and capsid are well known in the art. rAAV of the present disclosure can be produced and isolated according to any appropriate method, e.g., methods described in Clément and Grieger (2016), Grieger et al. (2016), and Martin et al. (2013), the contents of which are incorporated herein by reference in their entirety. Without wishing to be bound by any particular theory or process, the methods typically involve culturing a host cell which contains a nucleic acid sequence encoding an AAV capsid protein or fragment thereof; a functional rep gene; a recombinant AAV vector composed of AAV ITRs, and an inhibitory nucleic acid or transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component or components under the control of an inducible promoter. However, the required component or components may be under the control of a constitutive promoter. Examples of suitable inducible and constitutive promoters are provided herein. In still another alternative, a selected stable host cell may contain a selected component or components under the control of a constitutive promoter and other selected component or components under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene and/or inhibitory nucleic acid) to be packaged into AAV particles, an AAV helper function vector, and an accessory function vector. An AAV helper function vector encodes the “AAV helper function” sequences (e.g., rep and cap), which function in trans for productive AAV replication and encapsidation. In some embodiments, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Recombinant Viral Vector Particles

The present disclosure, among other things, provides methods, compositions, and systems for producing recombinant viral vector particles (e.g., recombinant adeno-associated viral (AAV) particles, or rAAV particles). In some embodiments, an rAAV particle may comprise an AAV genome and a capsid. In some embodiments, an rAAV particle may comprise a modified AAV genome comprising (i) a promoter, and (ii) at least one miRNA sequence; and a capsid. In some embodiments, an rAAV particle may comprise a modified AAV genome comprising (i) a promoter, and (ii) at least two or more different miRNA sequences; and a capsid. Recombinant viral vectors have become widely used for inserting genes into mammalian cells (e.g., human cells). Many forms of viral vectors can be used to deliver a payload (e.g., a payload described herein) to a cell, tissue, or organism.

Non-limiting examples of recombinant viral vectors include, but are not limited to, adeno-associated virus (AAV), retrovirus (e.g., Moloney murine leukemia virus (MMLV), Harvey murine sarcoma virus, murine mammary tumor virus, or Rous sarcoma virus), adenovirus, SV40-type virus, polyomavirus, Epstein-Barr virus, papilloma virus, herpes virus, vaccinia virus, or polio virus.

In some embodiments, a recombinant viral vector comprises or is a retroviral vector. Retroviruses are enveloped viruses that belong to viral family Retroviridae. Protocols for production of replication-deficient retroviruses are known in the art (See, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991), each of which is hereby incorporated by reference in its entirety). A number of retroviral systems are known in the art (See, e.g., U.S. Pat. Nos. 5,994,136, 6,165,782, and 6,428,953, each of which is hereby incorporated by reference in its entirety). In some embodiments, a retrovirus comprises or is a lentivirus of Retroviridae family. In some embodiments, a lentivirus comprises or is human immunodeficiency viruses (e.g., HIV-1 or HIV-2), simian immunodeficiency virus (S1V), feline immunodeficiency virus (FIV), equine infections anemia (EIA), or visna virus.

In some embodiments, a recombinant viral vector comprises or is an adenovirus vector. An adenovirus vector may be from any origin, subgroup, subtype, serotype, or mixture thereof. For instance, an adenovirus can be of subgroup A (e.g., serotypes 12, 18, or 31), subgroup B (e.g., serotypes 3, 7, 11, 14, 16, 21, 34, 35, or 50), subgroup C (e.g., serotypes 1, 2, 5, or 6), subgroup D (e.g., serotypes 8, 9, 10, 13, 15, 17, 19, 20, 22-30, 32, 33, 36-39, or 42-48), subgroup E (e.g., serotype 4), subgroup F (e.g., serotypes 40 or 41), an unclassified serogroup (e.g., serotypes 49 or 51), or any other adenoviral serotype. Adenoviral serotypes 1 through 51 are available from the American Type Culture Collection (ATCC, Manassas, Va.).

Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087, each of which is hereby incorporated by reference in its entirety. Further examples of adenoviral vectors can be found in U.S. Publication Nos. 20150093831, 20140248305, 20120283318, 20100008889, 20090175897 and 20090088398, each of which is hereby incorporated by reference in its entirety.

In some embodiments, a recombinant viral vector comprises or is an alphavirus. Exemplary alphaviruses include, but are not limited to, Sindbis virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, a genome of such viruses encodes nonstructural (e.g., replicon) and structural proteins (e.g., capsid and envelope) that can be translated in host cell cytoplasm. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819, each of which is incorporated herein by reference in their entirety

In some embodiments, a recombinant viral vector comprises or is an AAV vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is hereby incorporated by reference in its entirety). Methods for generating and using AAV vectors are described, for example, in U.S. Pat. Nos. 5,139,941 and 4,797,368, each of which is hereby incorporated by reference in its entirety.

Generally, AAV vectors for use in methods, compositions, and systems described herein may be of any AAV serotype. AAV serotypes generally have different tropisms to infect different tissues. In some embodiments, an AAV serotype is selected based on a tropism. Several AAV serotypes have been characterized including, but not limited to, AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, as well as variants or hybrids thereof. For example, in some embodiments, an AAV vector comprises or is an AAV2/5, AAV2/6, AAV2/8 or AAV2/9 vector (e.g., AAV6, AAV8 or AAV9 serotype having AAV2 ITR).

In some embodiments, an AAV vector is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, an AAV serotype may have or comprise a mutation in an AAV9 sequence (e.g., as described in N Pulicherla et al. Molecular Therapy 19(6): 1070-1078 (2011), which is hereby incorporated by reference in its entirety). AAV9 serotypes may include, but not limited to, AAV9.68, AAV9.9, AAV9.11, AAV9.13, AAV9.16, AAV9.24, AAV9.45, AAV9.47, AAV9.61, and AAV9.84. In certain embodiments, an AAV9 variant comprises or is AAVhu68 or a variant thereof (e.g., as described in WO 2018/160585, which is hereby incorporated by reference in its entirety). Other AAV vectors are described in, e.g., Sharma et al., Brain Res Bull. 2010 Feb. 15; 81(2-3): 273, which is hereby incorporated by reference in its entirety.

In some embodiments, an AAV vector comprises or is a naturally occurring AAV. In some embodiments, an AAV vector is a modified AAV or a variant of a naturally occurring AAV. In some embodiments, an AAV vector may be generated by directed evolution, e.g., by DNA shuffling, peptide insertion, or random mutagenesis, in order to introduce modifications into the AAV sequence to improve one or more properties for gene therapy. In some embodiments, such modifications avoid or lessen an immune response or recognition by neutralizing antibodies and/or allow for more efficient and/or targeted transduction (See, e.g., Asuri et al., Molecular Therapy 20.2 (2012): 329-338, which is hereby incorporated by reference in its entirety). Methods of using directed evolution to engineer an AAV vector can be found, e.g., in U.S. Pat. No. 8,632,764, which is hereby incorporated by reference in its entirety. In some embodiments, a modified AAV is modified to include a specific tropism.

In some embodiments, an AAV vector may be a dual or triple AAV vector, e.g., for the delivery of large payloads (e.g., payloads of greater than approximately 5 kb) and/or to address safety concerns associated with administration of single AAV vectors. In some embodiments, a dual AAV vector may include two separate AAV vectors, each including a fragment of a full sequence of a large payload of interest, and when recombined, the fragments form the full sequence of the large payload of interest or a functional portion thereof. In some embodiments, a triple AAV vector may include three separate AAV vectors, each including a fragment of a sequence of a large payload of interest, and when recombined, the fragments form the full sequence of the large payload of interest or a functional portion thereof.

Multiple AAV (e.g., dual or triple AAV vectors) can be delivered to and co-transduced into the same cell, where fragments of a payload of interest recombine and generate a single mRNA transcript of the entire payload of interest. In some embodiments, fragmented payloads include a non-overlapping sequences. In some embodiments, fragmented payloads include a specified overlapping sequences. In some embodiments, multiple AAV vectors for dual or triple transfection may be the same type of AAV vector (e.g., same serotype and/or same construct). In some embodiments, multiple AAV vectors of dual or triple may transfection be different types of AAV vector (e.g., different serotype or different construct).

In some embodiments, an AAV vector comprises a single-stranded (ss) or self-complementary (sc) AAV nucleic acid vector. In some embodiments, an AAV vector comprises an expression construct and one or more regions comprising ITR sequences (e.g., wild-type ITR sequences or engineered ITR sequences) flanking an expression construct. In some embodiments, an AAV vector is encapsidated by a viral capsid. In some embodiments, a viral capsid comprises 60 capsid protein subunits. In some embodiments, a viral capsid comprises VP1, VP2, and VP3. In some embodiments, VP1, VP2, and VP3 subunits are present in a capsid at a ratio of about 1:1:10, respectively.

ITR sequences of an AAV vector can be derived from any AAV serotype (e.g., AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, or variants or hybrids thereof). In some embodiments, ITR sequences are derived from one or more other serotypes, e.g., as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

ITR sequences and plasmids containing ITR sequences are known in the art and are commercially available (See, e.g., products and services available from Vector Biolabs, Philadelphia, PA; Cellbiolabs, San Diego, CA; Agilent Technologies, Santa Clara, Ca; and Addgene, Cambridge, MA; and described in Kessler et al. PNAS. 1996 Nov. 26; 93(24): 14082-7; Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus; and U.S. Pat. Nos. 5,139,941 and 5,962,313; each of which is hereby incorporated by reference in its entirety).

An AAV vector may comprise or be based on a serotype selected from any following serotypes or variants thereof including, but not limited to, AAV9.68, AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and AAV SM 10-8.

An AAV serotype may be from any number of species. For example, an AAV may be or comprise an avian AAV (AAAV), e.g., as described in U.S. Pat. No. 9,238,800, which is hereby incorporated by reference in its entirety. An AAV serotype may be or comprise a bovine AAV (BAAV), e.g., as described in U.S. Pat. No. 9,193,769 or 7,427,396, each of which is hereby incorporated by reference in its entirety. An AAV may be or comprise a caprine AAV, e.g., as described in U.S. Pat. No. 7,427,396, which is hereby incorporated by reference in its entirety. An AAV serotype may also be a variant or hybrid of any of the foregoing.

In some embodiments, an AAV may be or comprise a serotype generated from an AAV9 capsid library with mutations in amino acids 390 to 627 (VP1 numbering), e.g., as described in Pulicherla et al. (Molecular Therapy 19(6): 1070-1078 (2011), which is hereby incorporated by reference in its entirety. An AAV serotype (with corresponding nucleotide and amino acid substitutions) may include, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (CI 531 A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A, G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L), and AAV9.95 (T1605A; F535L).

In some embodiments, an AAV vector comprises a capsid including modified capsid proteins (e.g., capsid proteins comprising a modified VP3 region). Methods of producing modified capsid proteins are known in the art (See, e.g., US20130310443, which is hereby incorporated by reference in its entirety). In some embodiments, an AAV vector comprises a modified capsid protein comprising at least one non-native amino acid substitution at a position that corresponds to a surface-exposed amino acid (e.g., a surface exposed tyrosine) in a wild-type capsid protein. In some embodiments, an AAV vector comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in a wild-type capsid protein, a non-lysine amino acid (e.g., a glutamic acid) at a position that corresponds to a surface-exposed lysine amino acid in a wild-type capsid protein, a non-serine amino acid (e.g., a valine) at a position that corresponds to a surface-exposed serine amino acid in a wild-type capsid protein, or a combination thereof. In some embodiments, an AAV vector comprises a capsid that includes modified capsid proteins having at least 1, 2, 3, 4, 5, 6, 7, 8, 9, 10, or more amino acid substitutions.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which are hereby incorporated by reference in their entirety.

### Methods of Use

The present disclosure provides, among other things, methods of treating a subject with ALS comprising a step of administering a therapeutically effective amount of inhibitory nucleic acids to said subject to inhibit expression of a gene that causes or is implicated in ALS pathogenesis. In some embodiments, the present disclosure provides methods of administering a therapeutically effective amount of one or more inhibitory nucleic acids that inhibit expression of SOD1. In some embodiments, methods of the present disclosure include methods of administering a therapeutically effective amount of two or more inhibitory nucleic acids that inhibit expression of SOD1. In some embodiments, the two or more inhibitory nucleic acids administered to a subject comprise or consist of different sequences. In some embodiments, inhibitory nucleic acids of the present disclosure are administered via recombinant AAV vectors. In some embodiments, methods of the present disclosure include methods of administering a therapeutically effective amount of a composition that provides a recombinant AAV vector that inhibits expression of a target nucleic acid. In some embodiments, methods of the present disclosure include methods of administering a therapeutically effective amount of a composition that provides a recombinant AAV vector that inhibits expression of SOD1. In some embodiments, inhibitory nucleic acids of the present disclosure comprise or consist of one or more RNA molecules that comprise one or more guide sequences that are complementary to a target nucleic acid (e.g., SOD1 mRNA) thereby facilitating inhibition of said target nucleic acid. In some embodiments, inhibitory nucleic acids of the present disclosure comprise or consist of one or more miRNAs. In some embodiments, inhibitory nucleic acids of the present disclosure comprise or consist of two or more miRNAs. In some preferred embodiments, methods of the present disclosure comprise a step of administering a recombinant AAV comprising a modified AAV genome comprising one or more miRNAs that target SOD1. In some preferred embodiments, methods of the present disclosure comprise a step of administering a recombinant AAV comprising a modified AAV genome comprising two or more miRNAs that target SOD1.

In some embodiments, methods of the present disclosure comprise recombinant AAV vectors comprising a modified AAV genome comprising a transgene or inhibitory nucleic acid flanked by ITR sequences, where ITR sequences can be from any AAV serotype. In some embodiments, ITR sequences of the present disclosure can include ITR sequences from the group consisting of AAV1, AAV2, AAV3, AAV4, AAV5, AAV6, AAV7, AAV8, AAV9, AAV10, AAV11, AAV12, or any combinations thereof. In some embodiments, ITR sequences of the present disclosure may comprise engineered or modified ITR sequences using methods known in the art.

The present disclosure provides methods comprising a step of administering inhibitory nucleic acids for treatment of ALS, where said inhibitory nucleic acids can be operably linked to any promoter that facilitates transcription of the inhibitory nucleic acid. In some embodiments, inhibitory nucleic acids of the present disclosure are operably linked to a constitutive or inducible promoter. In some embodiments, inhibitory nucleic acids of the present disclosure are operably linked to promoters selected from the group consisting of CMV, EF1a, SV40, PGK, PGK1, Ubc, human beta-actin, beta-actin long (BActL), CAG, CBA, CBh, TRE, U6, H1, 7SK, ubiquitin C (UbiC), and any combinations thereof. In some embodiments, inhibitory nucleic acids of the present disclosure are operably linked to a modified or engineered promoter. In some embodiments inhibitory nucleic acids of the present disclosure are operably linked to tissue or cell specific promoters to enable targeting of a subset of tissues or cells that are particularly affected in a disease or disorder of interest (e.g., ALS). In some embodiments, inhibitory nucleic acids of the present disclosure are operably linked to one or more promoters as described herein.

In some embodiments of the present disclosure, inhibitory nucleic acids may be operably linked to 5′ regulatory elements and/or 3′ regulatory elements. In some embodiments, of the present disclosure, inhibitory nucleic acids may also comprise intronic sequences. In some embodiments, inhibitory nucleic acids may comprise 5′ untranslated and 3′ untranslated regions as required. In some embodiments, the present disclosure provides inhibitory nucleic acids comprising sequences involved with transcription such as TATA box, capping sequences, CAAT sequences, enhancer elements, IRES, and combinations thereof. In some embodiments of the present disclosure, 3′ regulatory elements may be selected from the group consisting of poly-A tails, AU-rich elements, and combinations thereof. In some embodiments, sequences involved with transcription include WPRE and P2A.

In some additional embodiments of the present disclosure, recombinant AAV may comprise reporter protein sequences that are operably linked to a promoter. In some embodiments, reporter protein sequences may be green fluorescent protein (GFP) or any variants thereof. In some embodiments, report protein sequences may be a luciferase protein or any variants thereof.

The present disclosure provides methods of treating a subject with Amyotrophic Lateral Sclerosis (ALS), the method comprising a step of: administering a therapeutically effective amount of a composition that provides a rAAV vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

The present disclosure further provides methods for simultaneously delivering two or more anti-SOD1 miRNAs to CNS tissue in a subject, the method comprising a step of: administering a therapeutically effective amount of a composition that provides a recombinant adeno-associated virus (rAAV) vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

The present disclosure provides method of inhibiting SOD1 expression in a cell, the method comprising a step of: administering a composition that provides a recombinant adeno-associated virus (rAAV) vector, wherein the rAAV vector comprises: (a) a modified AAV genome comprising: (i) a promoter; and (ii) two or more different miRNA sequences; and (b) a capsid; wherein each of the two or more miRNA sequences comprise a guide strand that targets SOD1, and a scaffold sequence, and wherein each of the two or more miRNA sequences are operably linked to the promoter.

The present disclosure provides methods of treating a subject with Amyotrophic Lateral Sclerosis (ALS), the method comprising co-administering: (i) a therapeutically effective amount of a composition that provides a rAAV particle provided herein; and (ii) one or more immunosuppressants. In some embodiments, an immunosuppressant may be selected from the group consisting of Abrocitinib, Baricitinib, Cyclosporine, Dexamethoasone (Dex), intravenous immune globulin (IVIG), Methylprednisolone, Mycophenolate Mofetil (MMF), Prednisone, Rituximab, Ruxolitinib, Sirolimus (Rapamycin), Steroid, Tacrolimus (Tacro), Tofacitinib (Tofa), and Upadacitinib. In some embodiments, an immunosuppressant may be an inhibitor of Janus Kinase (JAK). In some embodiments, an immunosuppressant may be administered before administration of an rAAV particle provided herein. In some embodiments, an immunosuppressant may be administered concurrently with an rAAV particle provided herein. In some embodiments, an immunosuppressant may be administered following administration of an rAAV particle provided herein. In some embodiments, the period of time between administration of an rAAV particle provided herein and an immunosuppressant may be at least 1 day, at least 1 week, at least 2 weeks, at least 3 weeks, at least 4 weeks, at least 6 weeks, at least 8 weeks, or at least 12 weeks, at least 6 months, or at least 1 year or more. In some embodiments, an immunosuppressant may be administered in multiple doses before and/or following administration of an rAAV particle provided herein. In some embodiments, an immunosuppressant may be administered for a period of at least 1 day, at least 1 week, at least 2 weeks, at least 1 month, at least 2 months, at least 3 months, at least 6 months, or at least 1 year following administration of an rAAV particle provided herein. In some embodiments, an immunosuppressant is administered for a period of at least 1 day, at least 1 week, at least 2 weeks, at least 1 month, at least 2 months, at least 3 months, at least 6 months, or at least 1 year before administration of an rAAV particle provided herein. In some embodiments, an immunosuppressant may be administered before and after administration of an rAAV particle provided herein.

Pharmaceutical Compositions

In general, compositions of the present disclosure may be administered in any form, including tablet, powder, or liquid, formulated into a pharmaceutically acceptable carrier or excipient, depending on the condition of the patient. Additionally, non-active ingredients well known in the art, such as binders, fillers, coatings, preservatives, coloring agents, flavoring agents and other additives may optionally be formulated with one or more administered agents, or left out completely if there is a risk of negative side effects to the patient such as increased the risk of intestinal inflammation or interference with the absorption of particular compounds.

Compositions of the present disclosure may be delivered to a subject according to any appropriate methods known in the art. In some embodiments, rAAV is administered to a subject at a dose of at least 10²⁰, at least 10¹⁸, at least 10¹⁶, at least 10¹⁴, at least 10¹², at least 10¹⁰, or at least 10⁸ genome copies per subject. In some embodiments, rAAV is administered to a subject at a dose of at most 10²⁰, at most 10¹⁸, at most 10¹⁶, at most 10¹⁴, at most 10¹², at most 10¹⁰, or at most 10⁸ genome copies per subject. In some embodiments, rAAV is administered to a subject at a dose within a range of about 10¹¹ to about 10¹⁶, 10¹¹ to about 10¹⁵, 10¹¹ to about 10¹⁴, 10¹¹ to about 10¹³, or 10¹¹ to about 10¹² genome copies per subject. In some embodiments, rAAV is administered to a subject at a dose within a range of about 10¹¹ to about 10¹³ genome copies per subject. In some embodiments, rAAV is administered to a subject at a dose within a range of about 10¹³ to about 10¹⁴ genome copies per subject. In some embodiments, rAAV is administered to a subject at a dose within a range of about 10¹³ to about 10¹⁵ genome copies per subject. In some embodiments, rAAV is administered to a subject at a dose within a range of about 10¹³ to about 10¹⁶ genome copies per subject.

Compositions of the present disclosure may be delivered to a subject according to any appropriate methods known in the art. In some embodiments, rAAV is administered to a subject at a dose of at least 10²⁰, at least 10¹⁸, at least 10¹⁶, at least 10¹⁴, at least 10¹², at least 10¹⁰, or at least 10⁸ genome copies per kg. In some embodiments, rAAV is administered to a subject at a dose of at most 10²⁰, at most 10¹⁸, at most 10¹⁶, at most 10¹⁴, at most 10¹², at most 10¹⁰, or at most 10⁸ genome copies per kg.

### Routes of Administration

Administration of compositions of the present disclosure may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal. In some embodiments, a preferred method of administration will reduce or prevent an immune response from a subject receiving treatment. In some embodiments, a preferred method of administration will reduce or prevent toxicity in a subject receiving treatment.

The present disclosure provides methods for treating ALS that exhibit reduced toxicity and/or immunoreactivity compared to compositions and methods known in the art. In some preferred embodiments of the present disclosure, methods of treating ALS that exhibit reduced toxicity and/or immunoreactivity comprise administration of inhibitory nucleic acids (e.g., in the form of rAAV) by intrathecal injection. In some embodiments, serum neurofilament (pNFH) measurement, and/or histopathological analysis of CNS tissues as well as peripheral organs, is used to assess the degree of toxicity of compositions and methods of the present disclosure and compositions and methods known in the art so they may be compared.

Formulations and compositions of the present disclosure may be prepared by any method known in the art of pharmacology. In general, such preparatory methods include the step of bringing the active ingredient into association with an excipient and/or one or more other accessory ingredients, or vehicles, and then, if necessary and/or desirable, dividing, shaping and/or packaging the product into a desired single- or multi-dose unit. In some embodiments, formulations and compositions of the present disclosure may be administered in a buffer (e.g., PBS). In some embodiments, formulations and compositions of the present disclosure may be administered in artificial cerebrospinal fluid (aCSF).

Sequence Listing
huSOD1-1
SEQ ID NO: 1
TCTGCTCGAAATTGATGATGC
huSOD1-2
SEQ ID NO: 2
ATTACTTTCCTTCTGCTCGAA
huSOD1-3
SEQ ID NO: 3
ATGAACATGGAATCCATGCAG
huSOD1-4
SEQ ID NO: 4
TTCAATAGACACATCGGCCAC
huSOD1-5
SEQ ID NO: 5
TACTTTCTTCATTTCCACCTT
huSOD1-6
SEQ ID NO: 6
TTTGTACTTTCTTCATTTCCA
huSOD1-7
SEQ ID NO: 7
TCAGGATACATTTCTACAGCT
huSOD1-8
SEQ ID NO: 8
TTATCAGGATACATTTCTACA
huSOD1-9
SEQ ID NO: 9
TTACAGTGTTTAATGTTTATC
huSOD1-10
SEQ ID NO: 10
TACACTTTTAAGATTACAGTG
huSOD1-11
SEQ ID NO: 11
AATGACAAAGAAATTCTGACA
huSOD1-12
SEQ ID NO: 12
TTTAGTTTGAATTTGGATTCT
LUC control target sequence
SEQ ID NO: 13
CCGGCTGAAGAGCCTGATCAA
REN control target sequence
SEQ ID NO: 14
AGGAATTATAATGCTTATCTA
CASI-emGFP-[inhibitory nucleic acid sequence]-WPRE
SEQ ID NO: 15
TTTAATTAAGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGTA
TCATATGCCAAGTCCGCCCCCTATTGACGTCAATGACGGTAAATGGCCCGCCTGGCATTATGC
CCAGTACATGACCTTACGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACC
CCCAATTTTGTATTTATTTATTTTTTAATTATTTTGTGCAGCGATGGGGGCGGGGGGGGGGGG
GGGGCGCGCGCCAGGCGGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGGCGAGGCGGCGGCGGCG
GCGGCCCTATAAAAAGCGAAGCGCGCGGCGGGCGGGAGTCGCTGCGACGCTGCCTTCGCCCCG
TGCCCCGCTCCGCCGCCGCCTCGCGCCGCCCGCCCCGGCTCTGACTGACCGCGTTACTAAAAC
AGGTAAGTCCGGCCTCCGCGCCGGGTTTTGGCGCCTCCCGCGGGCGCCCCCCTCCTCACGGCG
AGCGCTGCCACGTCAGACGAAGGGCGCAGGAGCGTTCCTGATCCTTCCGCCCGGACGCTCAGG
ACAGCGGCCCGCTGCTCATAAGACTCGGCCTTAGAACCCCAGTATCAGCAGAAGGACATTTTA
GGACGGGACTTGGGTGACTCTAGGGCACTGGTTTTCTTTCCAGAGAGCGGAACAGGCGAGGAA
AAGTAGTCCCTTCTCGGCGATTCTGCGGAGGGATCTCCGTGGGGCGGTGAACGCCGATGATGC
CTCTACTAACCATGTTCATGTTTTCTTTTTTTTTCTACAGGTCCTGGGTGACGAACAGACCGG
GAGCTCTCTGGCTAACTAGAGAACCCACTGCTTACTGGCTTATCGAAATTAATACGACTCACT
ATAGGGAGTCCCAAGCTGGCTAGTTAAGCTATCAACAAGTTTGTACAAAAAAGCAGGCTTTAA
AACCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCTGGCCCACCCTCGT
GACCACCTTCACCTACGGCGTGCAGTGCTTCGCCCGCTACCCCGACCACATGAAGCAGCACGA
CTTCTTCAAGTCCGCCATGCCCGAAGGCTACGTCCAGGAGCGCACCATCTTCTTCAAGGACGA
CGGCAACTACAAGACCCGCGCCGAGGTGAAGTTCGAGGGCGACACCCTGGTGAACCGCATCGA
GCTGAAGGGCATCGACTTCAAGGAGGACGGCAACATCCTGGGGCACAAGCTGGAGTACAACTA
CAACAGCCACAAGGTCTATATCACCGCCGACAAGCAGAAGAACGGCATCAAGGTGAACTTCAA
GACCCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCTGAG
CAAAGACCCCAACGAGAAGCGCGATCACATGGTCCTGCTGGAGTTCGTGACCGCCGCCGGGAT
CACTCTCGGCATGGACGAGCTGTACAAGTAAGCTAAGCACTTCGTGGCCGTCGATCGTTTAAA
GGGAGGTAGTGA-[inhibitory nucleic acid sequence]-AGCTCGCTGATCATA
ATCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGTTG
TCAGGCAACGTGGCGTGGTGTGCACTGTGTTTGCTGACGCAACCCCCACTGGTTGGGGCATTG
CCACCACCTGTCAGCTCCTTTCCGGGACTTTCGCTTTCCCCCTCCCTATTGCCACGGCGGAAC
TCATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCACCTGGATTC
TGCGCGGGACGTCCTTCTGCTACGTCCCTTCGGCCCTCAATCCAGCGGACCTTCCTTCCCGCG
GCCTGCTGCCGGCTCTGCGGCCTCTTCCGCGTCTTCGCCTTCGCCCTCAGACGAGTCGG
ATCTCCCTTTGGGCCGCCTCCCCGCTGATC
miR-155
SEQ ID NO: 16
CTGGAGGCTTGCTGAAGGCTGTATGCTG-[guide/passenger strand ~15-30bp]-
GTTTTGGCCACTGACTGAC-[guide/passenger strand ~15-30bp]-CAGGACACA
AGGCCTGTTACTAGCACTCACATGGAACAAATGGCC
miR-E
SEQ ID NO: 17
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CG-[guide/passenger strand ~15-30bp]-TAGTGAAGCCACAGATGTA-
[guide/passenger strand ~15-30bp]-ATGCCTACTGCCTCGGACTTCAAGGGGCT
AGAATTCGAGCAATTATCTTGTTTACTAAAACTGAATACCTTGCTATCTCTTTGATACATTTT
TACAAAGCTGAATTAAAATGGTATAAATTAAATCACTTT
ultramiR
SEQ ID NO: 18
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGC-[guide/passenger strand ~15-30bp]-TAGTGAAGCCACAGATGTA-
[guide/passenger strand ~15-30bp]-TTGCCTACTGCCTCGGACTTCAAGGGGCT
ACTTTAGGAGCAATTATCTTGTTTACTAAAACTGAATACCTTGCTATCTCTTTGATACATTTT
TACAAAGCTGAATTAAAATGGTATAAATTAAATCACTTTA
ultramiR + miR-155
SEQ ID NO: 19
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGC-[guide/passenger strand ~15-30bp]-TAGTGAAGCCACAGATGTA-
[guide/passenger strand ~15-30bp]-TTGCCTACTGCCTCGGACTTCAAGGGGCT
ACTTTAGGAGCAATTATCTTGTTTACTAAAACTGAATACCTTGCTATCTCTTTGATACATTTT
TACAAAGCTGAATTAAAATGGTATAAATTAAATCACTTTA-[optional spacer]-CTGG
AGGCTTGCTGAAGGCTGTATGCTG-[guide/passenger strand ~15-30bp]-GTTT
TGGCCACTGACTGAC-[guide/passenger strand ~15-30bp]-CAGGACACAAGGC
CTGTTACTAGCACTCACATGGAACAAATGGCC
miR-155 + ultramiR
SEQ ID NO: 20
CTGGAGGCTTGCTGAAGGCTGTATGCTG-[guide/passenger strand ~15-30bp]-
GTTTTGGCCACTGACTGAC-[guide/passenger strand ~15-30bp]-CAGGACACA
AGGCCTGTTACTAGCACTCACATGGAACAAATGGCC-[optional spacer]-TGTTTGAA
TGAGGCTTCAGTACTTTACAGAATCGTTGCCTGCACATCTTGGAAACACTTGCTGGGATTACT
TCTTCAGGTTAACCCAACAGAAGGCTAAAGAAGGTATATTGCTGTTGACAGTGAGCGC-
[guide/passenger strand ~15-30bp]-TAGTGAAGCCACAGATGTA-[guide/
passenger strand ~15-30bp]-TTGCCTACTGCCTCGGACTTCAAGGGGCTACTTTAG
GAGCAATTATCTTGTTTACTAAAACTGAATACCTTGCTATCTCTTTGATACATTTTTACAAAG
CTGAATTAAAATGGTATAAATTAAATCACTTTA
miR-155 + miR-E
SEQ ID NO: 21
CTGGAGGCTTGCTGAAGGCTGTATGCTG-[guide/passenger strand ~15-30bp]-
GTTTTGGCCACTGACTGAC-[guide/passenger strand ~15-30bp]-CAGGACACA
AGGCCTGTTACTAGCACTCACATGGAACAAATGGCC-[optional spacer]-TGTTTGAA
TGAGGCTTCAGTACTTTACAGAATCGTTGCCTGCACATCTTGGAAACACTTGCTGGGATTACT
TCGACTTCTTAACCCAACAGAAGGCTCGAGAAGGTATATTGCTGTTGACAGTGAGCG-
[guide/passenger strand ~15-30bp]-TAGTGAAGCCACAGATGTA-[guide/
passenger strand ~15-30bp]-ATGCCTACTGCCTCGGACTTCAAGGGGCTAGAATTC
GAGCAATTATCTTGTTTACTAAAACTGAATACCTTGCTATCTCTTTGATACATTTTTACAAAG
CTGAATTAAAATGGTATAAATTAAATCACTTT
miR-155 huSOD1-2
SEQ ID NO: 22
CTGGAGGCTTGCTGAAGGCTGTATGCTGATTACTTTCCTTCTGCTCGAAGTTTTGGCCACTGA
CTGACTTCGAGCAAGGAAAGTAATCAGGACACAAGGCCTGTTACTAGCACTCACATGGAACAA
ATGGCC
miR-155 huSOD1-3
SEQ ID NO: 23
CTGGAGGCTTGCTGAAGGCTGTATGCTGATGAACATGGAATCCATGCAGGTTTTGGCCACTGA
CTGACCTGCATGGTCCATGTTCATCAGGACACAAGGCCTGTTACTAGCACTCACATGGAACAA
ATGGCC
miR-155 huSOD1-5
SEQ ID NO: 24
CTGGAGGCTTGCTGAAGGCTGTATGCTGTACTTTCTTCATTTCCACCTTGTTTTGGCCACTGA
CTGACAAGGTGGATGAAGAAAGTACAGGACACAAGGCCTGTTACTAGCACTCACATGGAACAA
ATGGCC
miR-155 huSOD1-7
SEQ ID NO: 25
CTGGAGGCTTGCTGAAGGCTGTATGCTGTCAGGATACATTTCTACAGCTGTTTTGGCCACTGA
CTGACAGCTGTAGATGTATCCTGACAGGACACAAGGCCTGTTACTAGCACTCACATGGAACAA
ATGGCC
miR-155 huSOD1-8
SEQ ID NO: 26
CTGGAGGCTTGCTGAAGGCTGTATGCTGTTATCAGGATACATTTCTACAGTTTTGGCCACTGA
CTGACTGTAGAAATATCCTGATAACAGGACACAAGGCCTGTTACTAGCACTCACATGGAACAA
ATGGCC
miR-155 huSOD1-9
SEQ ID NO: 27
CTGGAGGCTTGCTGAAGGCTGTATGCTGTTACAGTGTTTAATGTTTATCGTTTTGGCCACTGA
CTGACGATAAACAAAACACTGTAACAGGACACAAGGCCTGTTACTAGCACTCACATGGAACAA
ATGGCC
miR-E huSOD1-2
SEQ ID NO: 28
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGCTTCGAGCAGAAGGAAAGTAATTAGTGAAGCCACAGATGTAATTACTTTCCTTCTGCTCGA
aATGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
miR-E huSOD1-3
SEQ ID NO: 29
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGACTGCATGGATTCCATGTTCATTAGTGAAGCCACAGATGTAATGAACATGGAATCCATGCA
GGTGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
miR-E huSOD1-5
SEQ ID NO: 30
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGCAAGGTGGAAATGAAGAAAGTATAGTGAAGCCACAGATGTATACTTTCTTCATTTCCACCT
tTTGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
miR-E huSOD1-7
SEQ ID NO: 31
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGCAGCTGTAGAAATGTATCCTGATAGTGAAGCCACAGATGTATCAGGATACATTTCTACAGC
TATGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
miR-E huSOD1-8
SEQ ID NO: 32
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGATGTAGAAATGTATCCTGATAATAGTGAAGCCACAGATGTATTATCAGGATACATTTCTAC
AGTGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
miR-E huSOD1-9
SEQ ID NO: 33
TGTTTGAATGAGGCTTCAGTACTTTACAGAATCGTTGCCTGCACATCTTGGAAACACTTGCTG
GGATTACTTCGACTTCTTAACCCAACAGAAGGCTCGAGAAGGTATATTGCTGTTGACAGTGAG
CGCGATAAACATTAAACACTGTAATAGTGAAGCCACAGATGTATTACAGTGTTTAATGTTTAT
CATGCCTACTGCCTCGGACTTCAAGGGGCTAGAATTCGAGCAATTATCTTGTTTACTAAAACT
GAATACCTTGCTATCTCTTTGATACATTTTTACAAAGCTGAATTAAAATGGTATAAATTAAAT
CACTTT
ultramiR huSOD1-2
SEQ ID NO: 34
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGCTTCGAGCAGAAGGAAAGTAAATAGTGAAGCCACAGATGTATTTACTTTCCTTCTGCTCGA
AATGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
ultramiR huSOD1-3
SEQ ID NO: 35
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGACTGCATGGATTCCATGTTCATTAGTGAAGCCACAGATGTAATGAACATGGAATCCATGCA
GGTGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
ultramiR huSOD1-5
SEQ ID NO: 36
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGCAAGGTGGAAATGAAGAAAGTATAGTGAAGCCACAGATGTATACTTTCTTCATTTCCACCT
TTTGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
ultramiR huSOD1-7
SEQ ID NO: 37
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGCAGCTGTAGAAATGTATCCTGATAGTGAAGCCACAGATGTATCAGGATACATTTCTACAGC
TATGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
ultramiR huSOD1-8
SEQ ID NO: 38
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGATGTAGAAATGTATCCTGATAATAGTGAAGCCACAGATGTATTATCAGGATACATTTCTAC
AGTGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
ultramiR huSOD1-9
SEQ ID NO: 39
TGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAG
CGCGATAAACATTAAACACTGTAATAGTGAAGCCACAGATGTATTACAGTGTTTAATGTTTAT
CATGCCTACTGCCTCGGACTTCAAGGGGCTACTTTAGGAGCAATTATCTTGTTTACTAAAACT
GAATACCTTGCTATCTCTTTGATACATTTTTACAAAGCTGAATTAAAATGGTATAAATTAAAT
CACTTTA
CAGG promoter full sequence
SEQ ID NO: 40
GACATTGATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAA
TTTTGTATTTATTTATTTTTTAATTATTTTGTGCAGCGATGGGGGCGGGGGGGGGGGGGGGGC
GCGCGCCAGGCGGGGCGGGGCGGGGCGAGGGGCGGGGCGGGGCGAGGCGGAGAGGTGCGGCGG
CAGCCAATCAGAGCGGCGCGCTCCGAAAGTTTCCTTTTATGGCGAGGCGGCGGCGGCGGCGGC
CCTATAAAAAGCGAAGCGCGCGGCGGGCGGGAGTCGCTGCGCGCTGCCTTCGCCCCGTGCCCC
GCTCCGCCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCGCTTGGTTTAATGACGGCTTGTTT
CTTTTCTGTGGCTGCGTGAAAGCCTTGAGGGGCTCCGGGAGGGCCCTTTGTGCGGGGGGAGCG
GCTCGGGGGGTGCGTGCGTGTGTGTGTGCGTGGGGAGCGCCGCGTGCGGCTCCGCGCTGCCCG
GCGGCTGTGAGCGCTGCGGGCGCGGCGCGGGGCTTTGTGCGCTCCGCAGTGTGCGCGAGGGGA
GCGCGGCCGGGGGCGGTGCCCCGCGGTGCGGGGGGGGCTGCGAGGGGAACAAAGGCTGCGTGC
GGGGTGTGTGCGTGGGGGGGTGAGCAGGGGGTGTGGGCGCGTCGGTCGGGCTGCAACCCCCCC
TGCACCCCCCTCCCCGAGTTGCTGAGCACGGCCCGGCTTCGGGTGCGGGGCTCCGTACGGGGC
GTGGCGCGGGGCTCGCCGTGCCGGGCGGGGGGTGGCGGCAGGTGGGGGTGCCGGGCGGGGCGG
GGCCGCCTCGGGCCGGGGAGGGCTCGGGGGAGGGGCGCGGCGGCCCCCGGAGCGCCGGCGGCT
GTCGAGGCGCGGCGAGCCGCAGCCATTGCCTTTTATGGTAATCGTGCGAGAGGGCGCAGGGAC
TTCCTTTGTCCCAAATCTGTGCGGAGCCGAAATCTGGGAGGCGCCGCCGCACCCCCTCTAGCG
GGCGCGGGGCGAAGCGGTGCGGCGCCGGCAGGAAGGAAATGGGCGGGGAGGGCCTTCGTGCGT
CGCCGCGCCGCCGTCCCCTTCTCCCTCTCCAGCCTCGGGGCTGTCCGCGGGGGGACGGCTGCC
TTCGGGGGGGACGGGGCAGGGCGGGGTTCGGCTTCTGGCGTGTGACCGGCGGCTCTAGAGCCT
CTGCTAACCATGTTCATGCCTTCTTCTTTTTCCTACAG
CMV enhancer
SEQ ID NO: 41
GACATTGATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCA
TG
Chicken Beta Actin promoter
SEQ ID NO: 42
TCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCG
CGCCAGGCGGGGCGGGGCGGGGCGAGGGGCGGGGCGGGGCGAGGCGGAGAGGTGCGGCGGCAG
CCAATCAGAGCGGCGCGCTCCGAAAGTTTCCTTTTATGGCGAGGCGGCGGCGGCGGCGGCCCT
ATAAAAAGCGAAGCGCGCGGCGGGCG
Chimeric Intron
SEQ ID NO: 43
GGAGTCGCTGCGCGCTGCCTTCGCCCCGTGCCCCGCTCCGCCGCCGCCTCGCGCCGCCCGCCC
CGGCTCTGACTGACCGCGTTACTCCCACAGGTGAGCGGGCGGGACGGCCCTTCTCCTCCGGGC
TGTAATTAGCGCTTGGTTTAATGACGGCTTGTTTCTTTTCTGTGGCTGCGTGAAAGCCTTGAG
GGGCTCCGGGAGGGCCCTTTGTGCGGGGGGAGCGGCTCGGGGGGTGCGTGCGTGTGTGTGTGC
GTGGGGAGCGCCGCGTGCGGCTCCGCGCTGCCCGGCGGCTGTGAGCGCTGCGGGCGCGGCGCG
GGGCTTTGTGCGCTCCGCAGTGTGCGCGAGGGGAGCGCGGCCGGGGGCGGTGCCCCGCGGTGC
GGGGGGGGCTGCGAGGGGAACAAAGGCTGCGTGCGGGGTGTGTGCGTGGGGGGGTGAGCAGGG
GGTGTGGGCGCGTCGGTCGGGCTGCAACCCCCCCTGCACCCCCCTCCCCGAGTTGCTGAGCAC
GGCCCGGCTTCGGGTGCGGGGCTCCGTACGGGGCGTGGCGCGGGGCTCGCCGTGCCGGGCGGG
GGGTGGCGGCAGGTGGGGGTGCCGGGCGGGGCGGGGCCGCCTCGGGCCGGGGAGGGCTCGGGG
GAGGGGCGCGGCGGCCCCCGGAGCGCCGGCGGCTGTCGAGGCGCGGCGAGCCGCAGCCATTGC
CTTTTATGGTAATCGTGCGAGAGGGCGCAGGGACTTCCTTTGTCCCAAATCTGTGCGGAGCCG
AAATCTGGGAGGCGCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCCCCTTCTCCCTCTC
CAGCCTCGGGGCTGTCCGCGGGGGGACGGCTGCCTTCGGGGGGGACGGGGCAGGGCGGGGTTC
GGCTTCTGGCGTGTGACCGGCGGCTCTAGAGCCTCTGCTAACCATGTTCATGCCTTCTTCTTT
TTCCTACAG
WPRE sequence
SEQ ID NO: 44
AATCAACCTCTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTT
GTCAGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTCC
GTGGTGTTGTCGGGGAAATCATCGTCCTTTCCTTGGCTGCTCGCCTGTGTTGCCACCTGGATT
CTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATC
TCCCTTTGGGCCGCCTCCCCGC
Human growth hormone polyA sequence
SEQ ID NO: 45
GGGTGGCATCCCTGTGACCCCTCCCCAGTGCCTCTCCTGGCCCTGGAAGTTGCCACTCCAGTG
CCCACCAGCCTTGTCCTAATAAAATTAAGTTGCATCATTTTGTCTGACTAGGTGTCCTTCTAT
AATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATC
TCCGCCTCCTGGGTTCAAGCGATTCTCCTGCCTCAGCCTCCCGAGTTGTTGGGATTCCAGGCA
TGCATGACCAGGCTCAGCTAATTTTTGTTTTTTTGGTAGAGACGGGGTTTCACCATATTGGCC
AGGCTGGTCTCCAACTCCTAATCTCAGGTGATCTACCCACCTTGGCCTCCCAAATTGCTGGGA
TTACAGGCGTGAACCACTGCTCCCTTCCCTGTCCTT
Homo sapiens superoxide dismutase 1 (SOD1),
mRNA (NM_000454.4)
SEQ ID NO: 46
GTTTGGGGCCAGAGTGGGCGAGGCGCGGAGGTCTGGCCTATAAAGTAGTCGCGGAGACGGGGT
GCTGGTTTGCGTCGTAGTCTCCTGCAGCGTCTGGGGTTTCCGTTGCAGTCCTCGGAACCAGGA
CCTCGGCGTGGCCTAGCGAGTTATGGCGACGAAGGCCGTGTGCGTGCTGAAGGGCGACGGCCC
AGTGCAGGGCATCATCAATTTCGAGCAGAAGGAAAGTAATGGACCAGTGAAGGTGTGGGGAAG
CATTAAAGGACTGACTGAAGGCCTGCATGGATTCCATGTTCATGAGTTTGGAGATAATACAGC
AGGCTGTACCAGTGCAGGTCCTCACTTTAATCCTCTATCCAGAAAACACGGTGGGCCAAAGGA
TGAAGAGAGGCATGTTGGAGACTTGGGCAATGTGACTGCTGACAAAGATGGTGTGGCCGATGT
GTCTATTGAAGATTCTGTGATCTCACTCTCAGGAGACCATTGCATCATTGGCCGCACACTGGT
GGTCCATGAAAAAGCAGATGACTTGGGCAAAGGTGGAAATGAAGAAAGTACAAAGACAGGAAA
CGCTGGAAGTCGTTTGGCTTGTGGTGTAATTGGGATCGCCCAATAAACATTCCCTTGGATGTA
GTCTGAGGCCCCTTAACTCATCTGTTATCCTGCTAGCTGTAGAAATGTATCCTGATAAACATT
AAACACTGTAATCTTAAAAGTGTAATTGTGTGACTTTTTCAGAGTTGCTTTAAAGTACCTGTA
GTGAGAAACTGATTTATGATCACTTGGAAGATTTGTATAGTTTTATAAAACTCAGTTAAAATG
TCTGTTTCAATGACCTGTATTTTGCCAGACTTAAATCACAGATGGGTATTAAACTTGTCAGAA
TTTCTTTGTCATTCAAGCCTGTGAATAAAAACCCTGTATGGCACTTATTATGAGGCTATTAAA
AGAATCCAAATTCAAACTAAAAAAAAAAAAAAAAA
Mus musculus superoxide dismutase 1,
soluble (Sod1), mRNA (NM_011434.2)
SEQ ID NO: 47
CGCGGTCCTTTCCTGCGGCGCCTTCCGTCCGTCGGCTTCTCGTCTTGCTCTCTCTGGTCCCTC
CGGAGGAGGCCGCCGCGCGTCTCCCGGGGAAGCATGGCGATGAAAGCGGTGTGCGTGCTGAAG
GGCGACGGTCCGGTGCAGGGAACCATCCACTTCGAGCAGAAGGCAAGCGGTGAACCAGTTGTG
TTGTCAGGACAAATTACAGGATTAACTGAAGGCCAGCATGGGTTCCACGTCCATCAGTATGGG
GACAATACACAAGGCTGTACCAGTGCAGGACCTCATTTTAATCCTCACTCTAAGAAACATGGT
GGCCCGGCGGATGAAGAGAGGCATGTTGGAGACCTGGGCAATGTGACTGCTGGAAAGGACGGT
GTGGCCAATGTGTCCATTGAAGATCGTGTGATCTCACTCTCAGGAGAGCATTCCATCATTGGC
CGTACAATGGTGGTCCATGAGAAACAAGATGACTTGGGCAAAGGTGGAAATGAAGAAAGTACA
AAGACTGGAAATGCTGGGAGCCGCTTGGCCTGTGGAGTGATTGGGATTGCGCAGTAAACATTC
CCTGTGTGGTCTGAGTCTCAGACTCATCTGCTACCCTCAAACCATTAAACTGTAATCTGAAGA
GTIGTAAAAAAAAAAAAAAAA
Macaca fascicularis mRNA, clone QmoA-14762
(similar to Homo sapiens superoxide
dismutase 1 (SOD1) (NM_000454.4))
SEQ ID NO: 48
TTTTGCGGCATAGTCTCCTGCAGCGTTTGCGGTCAGTCTCGCAATATTCGGAAGCAGGACCGC
GGCGTGGCCTAGCAAGTCATGGCGATGAAGGCCGTGTGCGTGTTGAAGGGCGACAGCCCAGTG
CAGGGCACCATCAATTTCGAGCAGAAGGAAAGTAATGGACCAGTGAAGGTGTGGGGAAGCATT
ACAGGATTGACTGAAGGCCTGCATGGATACCATGTTCATCAGTTTGGAGATAATACACAAGGC
TGTACCAGTGCAGGTCCTCACTTTAATCCTCTATCCAGACAACACGGTGGGCCAAAGGATGAA
GAGAGGCATGTTGGAGACCTGGGCAATGTGACTGCTGGCAAAGATGGTGTGGCCAAGGTGTCT
TTCGAAGATTCTGTGATCTCGCTCTCAGGAGACCATTCCATCATTGGCCGCACATTGGTGGTC
CATGAAAAAGCAGATGACTTGGGCAAAGGTGGAAATGAAGAAAGTAAAAAGACAGGAAACGCT
GGAGGTCGTCTGGCTTGTGGTGTAATTGGGATCGCCCATTAAACATTCCCTTGGATGTAGTCT
GAGGCCCATTAACTCATCTGTTATCCTGCTAGCTGTAGAAATGTATCTTGATAAACATTAAAC
ACTGTAATCTTAAGAGTGTAATTGTGTGACGTTTGCTTAGTACCTGTAATGAGAAACTGGTTG
ATGATCACTTGGAAGATTTGTATAGTTTTATAAAACTCAATTAAAATGTCTGTTTCAATGACC
TGTATTTTGCCAGACTTAATCACAGATGGGTATTAAACTTGTCGGACACATCTTCCTCCTCCC
CACCCGAGCCTGGAGCACTCTAACCCTTGGAGACCCCCTAAGCCCTGTTCCTCCAGAGACCGA
GGCCCTCCAGAAGGGCTGAGCGGGGATAGGCTTGCCTGAGCCTGGAGCTGGGCTTTGGGGCAG
CCTGCGACCCTCCCCACTTGTGCCCCTTCTCCTGGGATCTCTGTGTCTTCCCTTTTCTTTCTG
GGGCCAGGAAGTCAGCGTCAACTCCTAGGCCCCAGATGCAGGGGCCCGGAAACACCTGCTCTC
CCCTGAGCCCCAAATGCAGGGGCCTGGGAACACCGTGCTGTCACCTGAGCCTGGGGGTCCCAT
CCCAGGAAGAGGGGCTGTCTCAGGACCTGAGTCCTCAGGGGCCCCGCACATTCAATCTGAAGG
TGACCCTGGCCTGGCCGAAGCTGGAAGAGCCGTGGGGACGCAGCCAGTAAACAGAGCGTAAGG
CTCAGGTGCTGGTTGGTTAATCCGTTTCTGGAGGAAGAGTATGACCCCCACCTGTGATGGGGT
CCTTGTGTGGTGGGGACCGGGGCCAGTGGGCTCCAGACCGCATGCTTAACCCGTGGATGTGAA
ACCTGCAGCAGAGAAGGAAGGTCGCATGAGTCAGATCCCAGTCCAGTAGTCAGTGGAGGGTGA
GGGTGACCCCATCTGCTATTTTTGTGCCCATCCTCAGACAGCCATTTGGGGATGTGCCTATTA
GGGCTCCCTAAGAACTCAGATGCCCAGGAAGCCCAGCCCCTCAGGACGTACCCACACGCAGCC
TTCCCTTGACGCCTACGTTTCTGGGCACATGAGGCATCTTTCCTGGAACCCCGAGCCAGCCCT
GTCCCGCCCCAACGCAGCATGGCACTCAGGAGATACAGGCTGGATGTGGGGCGGTCCTTCTGG
GGAGGCCTGGCCTAGCAGCCTGCCCTCTGCACGCTGCCCACCTGAGCCCTCCCTGCCAGGCTT
CATGCTGGGGTGGGCCACATGCCAGGACAAGAGGACCCCAGCAGAAAGCCAGCCCCGGACTCA
CTTGGGTGTGTTAAAATGGCTTCTACCTACATACAACATGGTAAAAGGTGTGGAACGTTTGCT
TGAAAATAATTGGGGGTGGGGGAGTGGTGAGAGGGTGGGGATGGGAGGGTTCCTGGAATTGGT
TCTTTATCCTGATTAGATGTGAAGGCACTAATGCTGATTTCTAGTAGTAAAAAGAGCACCAAT
AGTCAAAAAAAAAAAAAAAAAA
Callithrix jacchus superoxide dismutase 1,
soluble (SOD1), mRNA (XM_002761360.4)
SEQ ID NO: 49
GAGCGCGCGCAGGGCGATTGGCTCCGGGCCAGAGTGGGTGGTGCACGTAGGTCCGGCCTATAA
AGTGCCCGCGGCGCTCTCGCTTGGGTTTGCGCCGTTCTCTTCTGCAGCGTCTGTGGTTTCTCT
GGCAGTCGTTGGAACCCGGATCCAGGCGTGGCCTCGCGAGTGATGGCGATGAAGGCGGTGTGC
GTGTTGAAGGGCGACGGCCCGGTGCAGGGCACCATCAATTTCGAGCAGAAGGAAAGTAATGGA
CCAGTTAAGGTGTGGGGAAGCATTACAGGATTGGCTGAAGGCCTGCATGGATTCCATGTTCAT
CAGTTTGGAGACAACACACAAGGCTGTACCAGTGCAGGTCCTCACTTTAATCCTCTATCCAGA
AAACATGGTGGGCCAGAGGATGAAGAGAGGCATGTTGGAGACCTGGGCAATGTGACTGCTGGT
AAAGATGGTGTGGCCAGTGTGTCAATTGAAGATTCTGTGATCTCACTCTCAGGAGTCCATTCC
ATCATTGGCCGCACGTTGGTGGTCCATGAAAAAGCAGATGACTTGGGCAAAGGTGGAAATGAA
GAAAGTACAAAGACAGGAAACGCTGGAAGTCGTTTGGCTTGTGGTGTCATTGGGATCGCCCAG
TAAACATTGCCCTGGATGTAGTCTGAGTCCCATTAACTCATCTGTTATCCTGGCTAGCTGTAG
AAATGTAACTTGACATTAAACACTGTAATCTTAAAAGCGTCATTTTAAGTGTGATTTTGAAAA
AAAAAGTTGCTTTAAAGTACCTCTAATGAGAAACTGGTTTATGATCACTTGGAAGATTTGTAT
AGTTTTATAAACCTCACATTAAAATGTTTCAGTGACCTGTA
Macaca mulatta superoxide dismutase 1 (SOD1),
mRNA (NM_001032804.1)
SEQ ID NO: 50
ATGGCGATGAAGGCCGTGTGCGTGTTGAAGGGCGACAGCCCAGTGCAGGGCACCATCAATTTC
GAGCAGAAGGAAAGTAATGGACCAGTGAAGGTGTGGGGAAGCATTACAGGATTGACTGAAGGC
CTGCATGGATTCCATGTTCATCAGTTTGGAGATAATACACAAGGCTGTACCAGTGCAGGTCCT
CACTTTAATCCTCTATCCAGACAACACGGTGGGCCAAAGGATGAAGAGAGGCATGTTGGAGAC
CTGGGCAATGTGACTGCTGGCAAAGATGGTGTGGCCAAGGTGTCTTTCGAAGATTCTGTGATC
TCGCTCTCAGGAGACCATTCCATCATTGGCCGCACATTGGTGGTCCATGAAAAAGCAGATGAC
TTGGGCAAAGGTGGAAATGAAGAAAGTAAAAAGACAGGAAACGCTGGAGGTCGTCTGGCTTGT
GGTGTAATTGGGATCGCCCAATAA
ultramiR huSOD1#5 + miR-155 huSOD1#7
(from ITR to ITR)
SEQ ID NO: 51
GCGGCCGGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCATC
GCTATTACCATGGTCGAGGTGAGCCCCACGTTCTGCTTCACTCTCCCCATCTCCCCCCCCTCC
CCACCCCCAATTTTGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGGGGCGAGGCGGAGA
GGTGCGGCGGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGCGCTGCCTTCGC
CCCGTGCCCCGCTCCGCCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCTTTGTGC
GGGGGGAGCGGCTCGGGGGGTGCGTGCGTGTGTGTGTGCGTGGGGAGCGCCGCGTGCGGCTCC
GCGCTGCCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGGGGAACAAA
GGCTGCGTGCGGGGTGTGTGCGTGGGGGGGTGAGCAGGGGGTGTGGGCGCGTCGGTCGGGCTG
CAACCCCCCCTGCACCCCCCTCCCCGAGTTGCTGAGCACGGCCCGGCTTCGGGTGCGGGGCTC
CGTACGGGGCGTGGCGCGGGGCTCGCCGTGCCGGGCGGGGGGTGGCGGCAGGTGGGGGTGCCG
GGCGGGGCGGGGCCGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTAATCGTGCGAGAGG
GCGCAGGGACTTCCTTTGTCCCAAATCTGTGCGGAGCCGAAATCTGGGAGGCGCCGCCGCACC
CCCTCTAGCGGGCGCGGGGCGAAGCGGTGCGGCGCCGGCAGGAAGGAAATGGGCGGGGAGGGC
CTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGGCTAGCGGTACCTGT
TTGAATGAGGCTTCAGTACTTTACAGAATCGTTGCCTGCACATCTTGGAAACACTTGCTGGGA
TTACTTCTTCAGGTTAACCCAACAGAAGGCTAAAGAAGGTATATTGCTGTTGACAGTGAGCGC
AAGGTGGAAATGAAGAAAGTATAGTGAAGCCACAGATGTATACTTTCTTCATTTCCACCTTTT
GCCTACTGCCTCGGACTTCAAGGGGCTACTTTAGGAGCAATTATCTTGTTTACTAAAACTGAA
TACCTTGCTATCTCTTTGATACATTTTTACAAAGCTGAATTAAAATGGTATAAATTAAATCAC
TTTAGCCTGGAGGCTTGCTGAAGGCTGTATGCTGTCAGGATACATTTCTACAGCTGTTTTGGC
CACTGACTGACAGCTGTAGATGTATCCTGACAGGACACAAGGCCTGTTACTAGCACTCACATG
GAACAAATGGCCGAGCTCAATCAACCTCTGGATTACAAAATTTGTGAAAGATTGACTGGTATT
CTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTAT
GAGGAGTTGTGGCCCGTTGTCAGGCAACGTGGCGTGGTGTGCACTGTGTTTGCTGACGCAACC
CCCACTGGTTGGGGCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGACAGGGGCTCGGCTG
TTGGGCACTGACAATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTCGGCCCTCAATCCA
GCGGACCTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCTGATCACGCCTAGGACGGGTGGC
ATCCCTGTGACCCCTCCCCAGTGCCTCTCCTGGCCCTGGAAGTTGCCACTCCAGTGCCCACCA
GCCTTGTCCTAATAAAATTAAGTTGCATCATTTTGTCTGACTAGGTGTCCTTCTATAATATTA
TGGGGTGGAGGGGGGTGGTATGGAGCAAGGGGCAAGTTGGGAAGACAACCTGTAGGGCCTGCG
GGGTCTATTGGGAACCAAGCTGGAGTGCAGTGGCACAATCTTGGCTCACTGCAATCTCCGCCT
CCTGGGTTCAAGCGATTCTCCTGCCTCAGCCTCCCGAGTTGTTGGGATTCCAGGCATGCATGA
CCAGGCTCAGCTAATTTTTGTTTTTTTGGTAGAGACGGGGTTTCACCATATTGGCCAGGCTGG
TCTCCAACTCCTAATCTCAGGTGATCTACCCACCTTGGCCTCCCAAATTGCTGGGATTACAGG
CGTGAACCACTGCTCCCTTCCCTGTCCTTACTAGTCGGCCGC
miR-155 huSOD1#2 + ultramiR huSOD1#5
(from ITR to ITR)
SEQ ID NO: 52
GCGGCCGGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCATC
GCTATTACCATGGTCGAGGTGAGCCCCACGTTCTGCTTCACTCTCCCCATCTCCCCCCCCTCC
CCACCCCCAATTTTGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGGGGCGAGGCGGAGA
GGTGCGGCGGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGCGCTGCCTTCGC
CCCGTGCCCCGCTCCGCCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCTTTGTGC
GGGGGGAGCGGCTCGGGGGGTGCGTGCGTGTGTGTGTGCGTGGGGAGCGCCGCGTGCGGCTCC
GCGCTGCCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGGGGAACAAA
GGCTGCGTGCGGGGTGTGTGCGTGGGGGGGTGAGCAGGGGGTGTGGGCGCGTCGGTCGGGCTG
CAACCCCCCCTGCACCCCCCTCCCCGAGTTGCTGAGCACGGCCCGGCTTCGGGTGCGGGGCTC
CGTACGGGGCGTGGCGCGGGGCTCGCCGTGCCGGGCGGGGGGTGGCGGCAGGTGGGGGTGCCG
GGCGGGGCGGGGCCGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTAATCGTGCGAGAGG
GCGCAGGGACTTCCTTTGTCCCAAATCTGTGCGGAGCCGAAATCTGGGAGGCGCCGCCGCACC
CCCTCTAGCGGGCGCGGGGCGAAGCGGTGCGGCGCCGGCAGGAAGGAAATGGGCGGGGAGGGC
CTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGGCTAGCGGTACCCTG
GAGGCTTGCTGAAGGCTGTATGCTGATTACTTTCCTTCTGCTCGAAGTTTTGGCCACTGACTG
ACTTCGAGCAAGGAAAGTAATCAGGACACAAGGCCTGTTACTAGCACTCACATGGAACAAATG
GCCGGTACCTGTTTGAATGAGGCTTCAGTACTTTACAGAATCGTTGCCTGCACATCTTGGAAA
CACTTGCTGGGATTACTTCTTCAGGTTAACCCAACAGAAGGCTAAAGAAGGTATATTGCTGTT
GACAGTGAGCGCAAGGTGGAAATGAAGAAAGTATAGTGAAGCCACAGATGTATACTTTCTTCA
TTTCCACCTTTTGCCTACTGCCTCGGACTTCAAGGGGCTACTTTAGGAGCAATTATCTTGTTT
ACTAAAACTGAATACCTTGCTATCTCTTTGATACATTTTTACAAAGCTGAATTAAAATGGTAT
AAATTAAATCACTTTAGAGCTCAATCAACCTCTGGATTACAAAATTTGTGAAAGATTGACTGG
TATTCTTAACTATGTTGCTCCTTTTACGCTATGTGGATACGCTGCTTTAATGCCTTTGTATCA
TGCTATTGCTTCCCGTATGGCTTTCATTTTCTCCTCCTTGTATAAATCCTGGTTGCTGTCTCT
TTATGAGGAGTTGTGGCCCGTTGTCAGGCAACGTGGCGTGGTGTGCACTGTGTTTGCTGACGC
AACCCCCACTGGTTGGGGCATTGCCACCACCTGTCAGCTCCTTTCCGGGACTTTCGCTTTCCC
CCTCCCTATTGCCACGGCGGAACTCATCGCCGCCTGCCTTGCCCGCTGCTGGACAGGGGCTCG
GCTGTTGGGCACTGACAATTCCGTGGTGTTGTCGGGGAAATCATCGTCCTTTCCTTGGCTGCT
CGCCTGTGTTGCCACCTGGATTCTGCGCGGGACGTCCTTCTGCTACGTCCCTTCGGCCCTCAA
TCCAGCGGACCTTCCTTCCCGCGGCCTGCTGCCGGCTCTGCGGCCTCTTCCGCGTCTTCGCCT
TCGCCCTCAGACGAGTCGGATCTCCCTTTGGGCCGCCTCCCCGCTGATCACGCCTAGGACGGG
TGGCATCCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCTATAAT
ATTATGGGGTGGAGGGGGGTGGTATGGAGCAAGGGGCAAGTTGGGAAGACAACCTGTAGGGCC
TGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGC
ATGACCAGGCTCAGCTAATTTTTGTTTTTTTGGTAGAGACGGGGTTTCACCATATTGGCCAGG
CTGGTCTCCAACTCCTAATCTCAGGTGATCTACCCACCTTGGCCTCCCAAATTGCTGGGATTA
CAGGCGTGAACCACTGCTCCCTTCCCTGTCCTTACTAGTCGGCCGC
miR-155 huSOD1#2 + miR-E huSOD1#7
(from ITR to ITR)
SEQ ID NO: 53
GCGGCCGGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCATC
GCTATTACCATGGTCGAGGTGAGCCCCACGTTCTGCTTCACTCTCCCCATCTCCCCCCCCTCC
CCACCCCCAATTTTGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGGGGCGAGGCGGAGA
GGTGCGGCGGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGCGCTGCCTTCGC
CCCGTGCCCCGCTCCGCCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCTTTGTGC
GGGGGGAGCGGCTCGGGGGGTGCGTGCGTGTGTGTGTGCGTGGGGAGCGCCGCGTGCGGCTCC
GCGCTGCCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGGGGAACAAA
GGCTGCGTGCGGGGTGTGTGCGTGGGGGGGTGAGCAGGGGGTGTGGGCGCGTCGGTCGGGCTG
CAACCCCCCCTGCACCCCCCTCCCCGAGTTGCTGAGCACGGCCCGGCTTCGGGTGCGGGGCTC
CGTACGGGGCGTGGCGCGGGGCTCGCCGTGCCGGGCGGGGGGTGGCGGCAGGTGGGGGTGCCG
GGCGGGGCGGGGCCGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTAATCGTGCGAGAGG
GCGCAGGGACTTCCTTTGTCCCAAATCTGTGCGGAGCCGAAATCTGGGAGGCGCCGCCGCACC
CCCTCTAGCGGGCGCGGGGCGAAGCGGTGCGGCGCCGGCAGGAAGGAAATGGGCGGGGAGGGC
CTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGGCTAGCGGTACCCTG
GAGGCTTGCTGAAGGCTGTATGCTGATTACTTTCCTTCTGCTCGAAGTTTTGGCCACTGACTG
ACTTCGAGCAAGGAAAGTAATCAGGACACAAGGCCTGTTACTAGCACTCACATGGAACAAATG
GCCGGTACCTGTTTGAATGAGGCTTCAGTACTTTACAGAATCGTTGCCTGCACATCTTGGAAA
CACTTGCTGGGATTACTTCGACTTCTTAACCCAACAGAAGGCTCGAGAAGGTATATTGCTGTT
GACAGTGAGCGCAGCTGTAGAAATGTATCCTGATAGTGAAGCCACAGATGTATCAGGATACAT
TTCTACAGCTATGCCTACTGCCTCGGACTTCAAGGGGCTAGAATTCGAGCAATTATCTTGTTT
ACTAAAACTGAATACCTTGCTATCTCTTTGATACATTTTTACAAAGCTGAATTAAAATGGTAT
AAATTAAATCACTTTGAGCTCAATCAACCTCTGGATTACAAAATTTGTGAAAGATTGACTGGT
ATTCTTAACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCTCAAT
CCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCTGATCACGCGCTAGGACGGG
TGGCATCCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCTATAAT
ATTATGGGGTGGAGGGGGGTGGTATGGAGCAAGGGGCAAGTTGGGAAGACAACCTGTAGGGCC
TGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGC
ATGACCAGGCTCAGCTAATTTTTGTTTTTTTGGTAGAGACGGGGTTTCACCATATTGGCCAGG
CTGGTCTCCAACTCCTAATCTCAGGTGATCTACCCACCTTGGCCTCCCAAATTGCTGGGATTA
CAGGCGTGAACCACTGCTCCCTTCCCTGTCCTTACTAGTCGGCCGC
mCherry
SEQ ID NO: 54
ATGGTGAGCAAGGGCGAGGAGGATAACATGGCCATCATCAAGGAGTTCATGCGCTTCAAGGTG
CACATGGAGGGCTCCGTGAACGGCCACGAGTTCGAGATCGAGGGCGAGGGCGAGGGCCGCCCC
TACGAGGGCACCCAGACCGCCAAGCTGAAGGTGACCAAGGGTGGCCCCCTGCCCTTCGCCTGG
GACATCCTGTCCCCTCAGTTCATGTACGGCTCCAAGGCCTACGTGAAGCACCCCGCCGACATC
CCCGACTACTTGAAGCTGTCCTTCCCCGAGGGCTTCAAGTGGGAGCGCGTGATGAACTTCGAG
GACGGCGGCGTGGTGACCGTGACCCAGGACTCCTCCCTGCAGGACGGCGAGTTCATCTACAAG
GTGAAGCTGCGCGGCACCAACTTCCCCTCCGACGGCCCCGTAATGCAGAAGAAGACCATGGGC
TGGGAGGCCTCCTCCGAGCGGATGTACCCCGAGGACGGCGCCCTGAAGGGCGAGATCAAGCAG
AGGCTGAAGCTGAAGGACGGCGGCCACTACGACGCTGAGGTCAAGACCACCTACAAGGCCAAG
AAGCCCGTGCAGCTGCCCGGCGCCTACAACGTCAACATCAAGTTGGACATCACCTCCCACAAC
GAGGACTACACCATCGTGGAACAGTACGAACGCGCCGAGGGCCGCCACTCCACCGGCGGCATG
GACGAGCTGTACAAGTAA
ultramiR huSOD1#5 + miR-155 huSOD1#7
(plasmidsequence)
SEQ ID NO: 55
cctgcaggcagctgcgcgctcgctcgctcactgaggccgcccgggcgtcgggcgacctttggt
cgcccggcctcagtgagcgagcgagcgcgcagagagggagtggccaactccatcactaggggt
tcctgcggccggtcgacattgattattgactagttattaatagtaatcaattacggggtcatt
agttcatagcccatatatggagttccgcgttacataacttacggtaaatggcccgcctggctg
accgcccaacgacccccgcccattgacgtcaataatgacgtatgttcccatagtaacgccaat
agggactttccattgacgtcaatgggtggagtatttacggtaaactgcccacttggcagtaca
tcaagtgtatcatatgccaagtacgccccctattgacgtcaatgacggtaaatggcccgcctg
gcattatgcccagtacatgaccttatgggactttcctacttggcagtacatctacgtattagt
catcgctattaccatggtcgaggtgagccccacgttctgcttcactctccccatctccccccc
ctccccacccccaattttgtatttatttattttttaattattttgtgcagcgatgggggcggg
gggggggggggggcgcgcgccaggcggggcggggggggcgaggggggggcggggcgaggcgga
gaggtgcggcggcagccaatcagagcggcgcgctccgaaagtttccttttatggcgaggcggc
ggcggcggcggccctataaaaagcgaagcgcgcggggggggagtcgctgcgcgctgccttcgc
cccgtgccccgctccgccgccgcctcgcgccgcccgccccggctctgactgaccgcgttactc
ccacaggtgagcgggcgggacggcccttctcctccgggctgtaattagcgcttggtttaatga
cggcttgtttcttttctgtggctgcgtgaaagccttgaggggctccgggagggccctttgtgc
ggggggagcggctcggggggtgcgtgcgtgtgtgtgtgcgtggggagcgccgcgtgcggctcc
gcgctgcccggcggctgtgagcgctgcgggcgcggcgcggggctttgtgcgctccgcagtgtg
cgcgaggggagcgcggccgggggcggtgccccgcggtgcgcaaccccccctgcacccccctcc
ccgagttgctgagcacggcccggcttcgggtgcggggctccgtacggggcgtggcgcggggct
cgccgtgccgggcggggggtggcggcaggtgggggtgccgggcggggggggccgcctcgggcc
ggggagggctcgggggaggggcgcggcggcccccggagcgccggcggctgtcgaggcgcggcg
agccgcagccattgccttttatggtaatcgtgcgagagggcgcagggacttcctttgtcccaa
atctgtgcggagccgaaatctgggaggcgccgccgcaccccctctagcgggcgcggggcgaag
cggtgcggcgccggcaggaaggaaatgggcggggagggccttcgtgcgtcgccgcgccgccgt
ccccttctccctctccagcctcggggctgtccgcggggggacggctgccttcgggggggacgg
ggcagggcggggttcggcttctggcgtgtgaccggcggctctagagcctctgctaaccatgtt
catgccttcttctttttcctacaggctagcggtaccTGTTTGAATGAGGCTTCAGTACTTTAC
AGAATCGTTGCCTGCACATCTTGGAAACACTTGCTGGGATTACTTCTTCAGGTTAACCCAACA
GAAGGCTAAAGAAGGTATATTGCTGTTGACAGTGAGCGCAAGGTGGAAATGAAGAAAGTATAG
TGAAGCCACAGATGTATACTTTCTTCATTTCCACCTTTTGCCTACTGCCTCGGACTTCAAGGG
GCTACTTTAGGAGCAATTATCTTGTTTACTAAAACTGAATACCTTGCTATCTCTTTGATACAT
TTTTACAAAGCTGAATTAAAATGGTATAAATTAAATCACTTTAgcctggaggcttgctgaagg
ctgtatgctgTCAGGATACATTTCTACAGCTgttttggccactgactgacAGCTGTAGATGTA
TCCTGAcaggacacaaggcctgttactagcactcacatggaacaaatggccgagctCAATCAA
CCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGTG
TTGTCGGGGAAATCATCGTCCTTTCCTTGGCTGCTCGCCTGTGTTGCCACCTGGATTCTGCGC
GGGACGTCCTTCTGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGTCGGATCTCCCTT
TGGGCCGCCTCCCCGCTGATCACGCCTAGGACGGGTGGCATCCCTGTGACCCCTCCCCAGTGC
CTCTCCTGGCCCTGGAAGTTGCCACTCCAGTGCCCACCAGCCTTGTCCTAATAAAATTAAGTT
GCATCATTTTGTCTGACTAGGTGTCCTTCTATAATATTATGGGGTGGAGGGGGGTGGTATGGA
GCAAGGGGCAAGTTGGGAAGACAACCTGTAGGGCCTGCGGGGTCTATTGGGAACCAAGCTGGA
GTGCAGTGGCACAATCTTGGCTCACTGCAATCTCCGCCTCCTGGGTTCAAGCGATTCTCCTGC
CTCAGCCTCCCGAGTTGTTGGGATTCCAGGCATGCATGACCAGGCTCAGCTAATTTTTGTTTT
TTTGGTAGAGACGGGGTTTCACCATATTGGCCAGGCTGGTCTCCAACTCCTAATCTCAGGTGA
TCTACCCACCTTGGCCTCCCAAATTGCTGGGATTACAGGCGTGAACCACTGCTCCCTTCCCTG
TCCTTActagtcggccgcaggaacccctagtgatggagttggccactccctctctgcgcgctc
gctcgctcactgaggccgggcgaccaaaggtcgcccgacgcccgggctttgcccgggcggcct
cagtgagcgagcgagcgcgcagctgcctgcaggggcgcctgatgcggtattttctccttacgc
atctgtgcggtatttcacaccgcatacgtcaaagcaaccatagtacgcgccctgtagcggcgc
attaagcgcggcgggtgtggtggttacgcgcagcgtgaccgctacacttgccagcgccttagc
gcccgctcctttcgctttcttcccttcctttctcgccacgttcgccggctttccccgtcaagc
tctaaatcgggggctccctttagggttccgatttagtgctttacggcacctcgaccccaaaaa
acttgatttgggtgatggttcacgtagtgggccatcgccctgatagacggtttttcgcccttt
gacgttggagtccacgttctttaatagtggactcttgttccaaactggaacaacactcaaccc
tatctcggtctattcttttgatttataagggattttgccgatttcggcctattggttaaaaaa
tgagctgatttaacaaaaatttaacgcgaattttaacaaaatattaacgcttacaatttaggt
ggcacttttcggggaaatgtgcgcggaacccctatttgtttatttttctaaatacattcaaat
atgtatccgctcatgagacaataaccctgataaatgcttcaataatattgaaaaaggaagagt
atgagtattcaacatttccgtgtcgcccttattcccttttttgcggcattttgccttcctgtt
tttgctcacccagaaacgctggtgaaagtaaaagatgctgaagatcagttgggtgcagtgtct
caaaatctctgatgttacattgcacaagataaaaatatatcatcatgaacaataaaactgtct
gcttacataaacagtaatacaaggggtgttatgagccatattcaacgggaaacgtcttgctcg
aggccgcgattaaattccaacatggatgctgatttatatgggtataaatgggctcgcgataat
gtcgggcaatcaggtgcgacaatctatcgattgtatgggaagcccgatgcgccagagttgttt
ctgaaacatggcaaaggtagcgttgccaatgatgttacagatgagatggtcagactaaactgg
ctgacggaatttatgcctcttccgaccatcaagcattttatccgtactcctgatgatgcatgg
ttactcaccactgcgatccccgggaaaacagcattccaggtattagaagaatatcctgattca
ggtgaaaatattgttgatgcgctggcagtgttcctgcgccggttgcattcgattcctgtttgt
aattgtccttttaacagcgatcgcgtatttcgtctcgctcaggcgcaatcacgaatgaataac
ggtttggttgatgcgagtgattttgatgacgagcgtaatggctggcctgttgaacaagtctgg
aaagaaatgcataagcttttgccattctcaccggattcagtcgtcactcatggtgatttctca
cttgataaccttatttttgacgaggggaaattaataggttgtattgatgttggacgagtcgga
atcgcagaccgataccaggatcttgccatcctatggaactgcctcggtgagttttctccttca
ttacagaaacggctttttcaaaaatatggtattgataatcctgatatgaataaattgcagttt
catttgatgctcgatgagtttttctaatcagaattggttaattggttgtaacactggcagagc
attacgctgacttgacgggacggcggctttgttgaataaatcgaacttttgctgagttgaagg
atcagatcacgcatcttcccgacaacgcagaccgttccgtggcaaagcaaaagttcaaaatca
ccaactggtccacctacaacaaagctctcatcaaccgtggctccctcactttctggctggatg
atggggcgattcaggcctggtatgagtcagcaacaccttcttcacgaggcagacctcagcgct
caaagatgcaggggtaaaagctaaccgcatctttaccgacaaggcatccggcagttcaacaga
tcgggaagggctggatttgctgaggatgaaggtggaggaaggtgatgtcattctggtgaagaa
gctcgaccgtcttggccgcgacaccgccgacatgatccaactgataaaagagtttgatgctca
gggtgtagcggttcggtttattgacgacgggatcagtaccgacggtgatatggggcaaatggt
ggtcaccaaggcctgctggtaatcaattgcctttttatttgggggagagggaagtcatgaaaa
aactaacctttgaaattcgatctccagcacatcagcaaaacgctattcacgcagtacagcaaa
tccttccagacccaaccaaaccaatcgtagtaaccattcaggaacgcaaccgcagcttagacc
aaaacaggaagctatgggcctgcttaggtgacgtctctcgtcaggttgaatggcatggtcgct
ggctggatgcagaaagctggaagtgtgtgtttaccgcagcattaaagcagcaggatgttgttc
ctaaccttgccgggaatggctttgtggtaataggccagtcaaccagcaggatgcgtgtaggcg
aatttgcggagctattagagcttatacaggcattcggtacagagcgtggcgttaagtggtcag
acgaagcgagactggctctggagtggaaagcgagatggggagacagggctgcatgataaatgt
cgttagtttctccggtggcaggacgtcagcatatttgctctggctaatggagcaaaagcgacg
ggcaggtaaagacgtgcattacgttttcatggatacaggttgtgaacatccaatgacatatcg
gtttgtcagggaagttgtgaagttctgggatataccgctcaccgtattgcaggttgatatcaa
cccggagcttggacagccaaatggttatacggtatgggaaccaaaggatattcagacgcgaat
gcctgttctgaagccatttatcgatatggtaaagaaatatggcactccatacgtcggcggcgc
gttctgcactgacagattaaaactcgttcccttcaccaaatactgtgatgaccatttcgggcg
agggaattacaccacgtggattggcatcagagctgatgaaccgaagcggctaaagccaaagcc
tggaatcagatatcttgctgaactgtcagactttgagaaggaagatatcctcgcatggtggaa
gcaacaaccattcgatttgcaaataccggaacatctcggtaactgcatattctgcattaaaaa
atcaacgcaaaaaatcggacttgcctgcaaagatgaggagggattgcagcgtgtttttaatga
ggtcatcacgggatcccatgtgcgtgacggacatcgggaaacgccaaaggagattatgtaccg
aggaagaatgtcgctggacggtatcgcgaaaatgtattcagaaaatgattatcaagccctgta
tcaggacatggtacgagctaaaagattcgataccggctcttgttctgagtcatgcgaaatatt
tggagggcagcttgatttcgacttcgggagggaagctgcatgatgcgatgttatcggtgcggt
gaatgcaaagaagataaccgcttccgaccaaatcaaccttactggaatcgatggtgtctccgg
tgtgaaagaacaccaacaggggtgttaccactaccgcaggaaaaggaggacgtgtggcgagac
agcgacgaagtatcaccgacataatctgcgaaaactgcaaataccttccaacgaaacgcacca
gaaataaacccaagccaatcccaaaagaatctgacgtaaaaaccttcaactacacggctcacc
tgtgggatatccggtggctaagacgtcgtgcgaggaaaacaaggtgattgaccaaaatcgaag
ttacgaacaagaaagcgtcgagcgagctttaacgtgcgctaactgcggtcagaagctgcatgt
gctggaagttcacgtgtgtgagcactgctgcgcagaactgatgagcgatccgaatagctcgat
gcacgaggaagaagatgatggctaaaccagcgcgaagacgatgtaaaaacgatgaatgccggg
aatggtttcaccctgcattcgctaatcagtggtggtgctctccagagtgtggaaccaagatag
cactcgaacgacgaagtaaagaacgcgaaaaagcggaaaaagcagcagagaagaaacgacgac
gagaggagcagaaacagaaagataaacttaagattcgaaaactcgccttaaagccccgcagtt
actggattaaacaagcccaacaagccgtaaacgccttcatcagagaaagagaccgcgacttac
catgtatctcgtgcggaacgctcacgtctgctcagtgggatgccggacattaccggacaactg
ctgcggcacctcaactccgatttaatgaacgcaatattcacaagcaatgcgtggtgtgcaacc
agcacaaaagcggaaatctcgttccgtatcgcgtcgaactgattagccgcatcgggcaggaag
cagtagacgaaatcgaatcaaaccataaccgccatcgctggactatcgaagagtgcaaggcga
tcaaggcagagtaccaacagaaactcaaagacctgcgaaatagcagaagtgaggccgcatgac
gttctcagtaaaaaccattccagacatgctcgttgaagcatacggaaatcagacagaagtagc
acgcagactgaaatgtagtcgcggtacggtcagaaaatacgttgatgataaagacgggaaaat
gcacgccatcgtcaacgacgttctcatggttcatcgcggatggagtgaaagagatgcgctatt
acgaaaaaattgatggcagcaaataccgaaatatttgggtagttggcgatctgcacggatgct
acacgaacctgatgaacaaactggatacgattggattcgacaacaaaaaagacctgcttatct
cggtgggcgatttggttgatcgtggtgcagagaacgttgaatgcctggaattaatcacattcc
cctggttcagagctgtacgtggaaaccatgagcaaatgatgattgatggcttatcagagcgtg
gaaacgttaatcactggctgcttaatggcggtggctggttctttaatctcgattacgacaaag
aaattctggctaaagctcttgcccataaagcagatgaacttccgttaatcatcgaactggtga
gcaaagataaaaaatatgttatctgccacgccgattatccctttgacgaatacgagtttggaa
agccagttgatcatcagcaggtaatctggaaccgcgaacgaatcagcaactcacaaaacggga
tcgtgaaagaaatcaaaggcgcggacacgttcatctttggtcatacgccagcagtgaaaccac
tcaagtttgccaaccaaatgtatatcgataccggcgcagtgttctgcggaaacctaacattga
ttcaggtacagggagaaggcgcatgagactcgaaagcgtagctaaatttcattcgccaaaaag
cccgatgatgagcgactcaccacgggccacggcttctgactctctttccggtactgatgtgat
ggctgctatggggatggcgcaatcacaagccggattcggtatggctgcattctgcggtaagca
cgaactcagccagaacgacaaacaaaaggctatcaactatctgatgcaatttgcacacaaggt
atcggggaaataccgtggtgtggcaaagcttgaaggaaatactaaggcaaaggtactgcaagt
gctcgcaacattcgcttatgcggattattgccgtagtgccgcgacgccgggggcaagatgcag
agattgccatggtacaggccgtgcggttgatattgccaaaacagagctgtgggggagagttgt
cgagaaagagtgcggaagatgcaaaggcgtcggctattcaaggatgccagcaagcgcagcata
tcgcgctgtgacgatgctaatcccaaaccttacccaacccacctggtcacgcactgttaagcc
gctgtatgacgctctggtggtgcaatgccacaaagaagagtcaatcgcagacaacattttgaa
tgcggtcacacgttagcagcatgattgccacggatggcaacatattaacggcatgatattgac
ttattgaataaaattgggtaaatttgactcaacgatgggttaattcgctcgttgtggtagtga
gatgaaaagaggcggcgcttactaccgattccgcctagttggtcacttcgacgtatcgtctgg
aactccaaccatcgcaggcagagaggtctgcaaaatgcaatcccgaaacagttcgcaggtaat
agttagagcctgcataacggtttcgggattttttatatctgcacaacaggtaagagcattgag
tcgataatcgtgaagagtcggcgagcctggttagccagtgctctttccgttgtgctgaattaa
gcgaataccggaagcagaaccggatcaccaaatgcgtacaggcgtcatcgccgcccagcaaca
gcacaacccaaactgagccgtagccactgtctgtcctgaattcattagtaatagttacgctgc
ggccttttacacatgaccttcgtgaaagcgggtggcaggaggtcgcgctaacaacctcctgcc
gttttgcccgtgcatatcggtcacgaacaaatctgattactaaacacagtagcctggatttgt
tctatcagtaatcgaccttattcctaattaaatagagcaaatccccttattgggggtaagaca
tgaagatgccagaaaaacatgacctgttggccgccattctcgcggcaaaggaacaaggcatcg
gggcaatccttgcgtttgcaatggcgtaccttcgcggcagatataatggcggtgcgtttacaa
aaacagtaatcgacgcaacgatgtgcgccattatcgcctggttcattcgtgaccttctcgact
tcgccggactaagtagcaatctcgcttatataacgagcgtgtttatcggctacatcggtactg
actcgattggttcgcttatcaaacgcttcgctgctaaaaaagccggagtagaagatggtagaa
atcaataatcaacgtaaggcgttcctcgatatgctggcgtggtcggagggaactgataacgga
cgtcagaaaaccagaaatcatggttatgacgtcattgtaggcggagagctatttactgattac
tccgatcaccctcgcaaacttgtcacgctaaacccaaaactcaaatcaacaggcgccaattgc
tggtcaccatcctgtcggctgtggcacaggctgaacgccggaggatcaaaaggatctaggtga
agatcctttttgataatctcatgaccaaaatcccttaacgtgagttttcgttccactgagcgt
cagaccccgtagaaaagatcaaaggatcttcttgagatcctttttttctgcgcgtaatctgct
gcttgcaaacaaaaaaaccaccgctaccagcggtggtttgtttgccggatcaagagctaccaa
ctctttttccgaaggtaactggcttcagcagagcgcagataccaaatactgttcttctagtgt
agccgtagttaggccaccacttcaagaactctgtagcaccgcctacatacctcgctctgctaa
tcctgttaccagtggctgctgccagtggcgataagtcgtgtcttaccgggttggactcaagac
gatagttaccggataaggcgcagcggtcgggctgaacggggggttcgtgcacacagcccagct
tggagcgaacgacctacaccgaactgagatacctacagcgtgagctatgagaaagcgccacgc
ttcccgaagggagaaaggcggacaggtatccggtaagcggcagggtcggaacaggagagcgca
cgagggagcttccagggggaaacgcctggtatctttatagtcctgtcgggtttcgccacctct
gacttgagcgtcgatttttgtgatgctcgtcaggggggcggagcctatggaaaaacgccagca
acgcggcctttttacggttcctggccttttgctggccttttgctcacatgt
miR-155 huSOD1#2 + ultramiR huSOD1#5
(plasmid sequence)
SEQ ID NO: 56
cctgcaggcagctgcgcgctcgctcgctcactgaggccgcccgggcgtcgggcgacctttggt
cgcccggcctcagtgagcgagcgagcgcgcagagagggagtggccaactccatcactaggggt
tcctgcggccggtcgacattgattattgactagttattaatagtaatcaattacggggtcatt
agttcatagcccatatatggagttccgcgttacataacttacggtaaatggcccgcctggctg
accgcccaacgacccccgcccattgacgtcaataatgacgtatgttcccatagtaacgccaat
agggactttccattgacgtcaatgggtggagtatttacggtaaactgcccacttggcagtaca
tcaagtgtatcatatgccaagtacgccccctattgacgtcaatgacggtaaatggcccgcctg
gcattatgcccagtacatgaccttatgggactttcctacttggcagtacatctacgtattagt
catcgctattaccatggtcgaggtgagccccacgttctgcttcactctccccatctccccccc
ctccccacccccaattttgtatttatttattttttaattattttgtgcagcgatgggggcggg
gggggggggggggcgcgcgccaggcggggggggcggggcgaggggggggcggggcgaggcgga
gaggtgcggcggcagccaatcagagcggcgcgctccgaaagtttccttttatggcgaggcggc
ggcggcggcggccctataaaaagcgaagcgcgcggggggggagtcgctgcgcgctgccttcgc
cccgtgccccgctccgccgccgcctcgcgccgcccgccccggctctgactgaccgcgttactc
ccacaggtgagcgggcgggacggcccttctcctccgggctgtaattagcgcttggtttaatga
cggcttgtttcttttctgtggctgcgtgaaagccttgaggggctccgggagggccctttgtgc
ggggggagcggctcggggggtgcgtgcgtgtgtgtgtgcgtggggagcgccgcgtgcggctcc
gcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaa
ggctgcgtgcggggtgtgtgcgtgggggggtgagcagggggtgggcgcgtcggtcgggctgca
accccccctgcacccccctccccgagttgctgagcacggcccggcttcgggtgcggggctccg
tacggggcgtggcgcggggctcgccgtgccgggcggggggtggcggcaggtgggggtgccggg
cggggggggccgcctcgggccggggagggctcgggggaggggcgcggcggcccccggagcgcc
ggcggctgtcgaggcgcggcgagccgcagccattgccttttatggtaatcgtgcgagagggcg
cagggacttcctttgtcccaaatctgtgcggagccgaaatctgggaggcgccgccgcaccccc
tctagcgggcgcggggcgaagcggtgcggcgccggcaggaaggaaatgggcggggagggcctt
cgtgcgtcgccgcgccgccgtccccttctccctctccagcctcggggctgtccgcggggggac
ggctgccttcgggggggacggggcagggcggggttcggcttctggcgtgtgaccggcggctct
agagcctctgctaaccatgttcatgccttcttctttttcctacaggctagcggtaccctggag
gcttgctgaaggctgtatgctgATTACTTTCCTTCTGCTCGAAgttttggccactgactgacT
TCGAGCAAGGAAAGTAATcaggacacaaggcctgttactagcactcacatggaacaaatggcc
ggtaccTGTTTGAATGAGGCTTCAGTACTTTACAGAATCGTTGCCTGCACATCTTGGAAACAC
TTGCTGGGATTACTTCTTCAGGTTAACCCAACAGAAGGCTAAAGAAGGTATATTGCTGTTGAC
AGTGAGCGCAAGGTGGAAATGAAGAAAGTATAGTGAAGCCACAGATGTATACTTTCTTCATTT
CCACCTTTTGCCTACTGCCTCGGACTTCAAGGGGCTACTTTAGGAGCAATTATCTTGTTTACT
AAAACTGAATACCTTGCTATCTCTTTGATACATTTTTACAAAGCTGAATTAAAATGGTATAAA
TTAAATCACTTTAgagctCAATCAACCTCTGGATTACAAAATTTGTGAAAGATTGACTGGTAT
TCTTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTTA
TGAGGAGTTGTGGCCCGTTGTCAGGCAACGTGGCGTGGTGTGCACTGTGTTTGCTGACGCAAC
CCCCACTGGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGGGCTCGGCT
GTTGGGCACTGACAATTCCGTGGTGTTGTCGGGGAAATCATCGTCCTTTCCTTGGCTGCTCGC
CTGTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTCG
CCCTCAGACGAGTCGGATCTCCCTTTGGGCCGCCTCCCCGCTGATCACGCCTAGGACGGGTGG
CATCCCTGTGACCCCTCCCCAGTGCCTCTCCTGGCCCTGGAAGTTGCCACTCCAGTGCCCACC
AGCCTTGTCCTAATAAAATTAAGTTGCATCATTTTGTCTGACTAGGTGTCCTTCTATAATATT
ATGGGGTGGAGGGGGGTGGTATGGAGCAAGGGGCAAGTTGGGAAGACAACCTGTAGGGCCTGC
GGGGTCTATTGGGAACCAAGCTGGAGTGCAGTGGCACAATCTTGGCTCACTGCAATCTCCGCC
TCCTGGGTTCAAGCGATTCTCCTGCCTCAGCCTCCCGAGTTGTTGGGATTCCAGGCATGCATG
ACCAGGCTCAGCTAATTTTTGTTTTTTTGGTAGAGACGGGGTTTCACCATATTGGCCAGGCTG
GTCTCCAACTCCTAATCTCAGGTGATCTACCCACCTTGGCCTCCCAAATTGCTGGGATTACAG
GCGTGAACCACTGCTCCCTTCCCTGTCCTTActagtcggccgcaggaacccctagtgatggag
ttggccactccctctctgcgcgctcgctcgctcactgaggccgggcgaccaaaggtcgcccga
cgcccgggctttgcccgggoggcctcagtgagcgagcgagcgcgcagctgcctgcaggggcgc
ctgatgcggtattttctccttacgcatctgtgcggtatttcacaccgcatacgtcaaagcaac
catagtacgcgccctgtagcggcgcattaagcgcggcgggtgtggtggttacgcgcagcgtga
ccgctacacttgccagcgccttagcgcccgctcctttcgctttcttcccttcctttctcgcca
cgttcgccggctttccccgtcaagctctaaatcgggggctccctttagggttccgatttagtg
ctttacggcacctcgaccccaaaaaacttgatttgggtgatggttcacgtagtgggccatcgc
cctgatagacggtttttcgccctttgacgttggagtccacgttctttaatagtggactcttgt
tccaaactggaacaacactcaaccctatctcggtctattcttttgatttataagggattttgc
cgatttcggcctattggttaaaaaatgagctgatttaacaaaaatttaacgcgaattttaaca
aaatattaacgcttacaatttaggtggcacttttcggggaaatgtgcgcggaacccctatttg
tttatttttctaaatacattcaaatatgtatccgctcatgagacaataaccctgataaatgct
tcaataatattgaaaaaggaagagtatgagtattcaacatttccgtgtcgcccttattccctt
ttttgcggcattttgccttcctgtttttgctcacccagaaacgctggtgaaagtaaaagatgc
tgaagatcagttgggtgcagtgtctcaaaatctctgatgttacattgcacaagataaaaatat
atcatcatgaacaataaaactgtctgcttacataaacagtaatacaaggggtgttatgagcca
tattcaacgggaaacgtcttgctcgaggccgcgattaaattccaacatggatgctgatttata
tgggtataaatgggctcgcgataatgtcgggcaatcaggtgcgacaatctatcgattgtatgg
gaagcccgatgcgccagagttgtttctgaaacatggcaaaggtagcgttgccaatgatgttac
agatgagatggtcagactaaactggctgacggaatttatgcctcttccgaccatcaagcattt
tatccgtactcctgatgatgcatggttactcaccactgcgatccccgggaaaacagcattcca
ggtattagaagaatatcctgattcaggtgaaaatattgttgatgcgctggcagtgttcctgcg
ccggttgcattcgattcctgtttgtaattgtccttttaacagcgatcgcgtatttcgtctcgc
tcaggcgcaatcacgaatgaataacggtttggttgatgcgagtgattttgatgacgagcgtaa
tggctggcctgttgaacaagtctggaaagaaatgcataagcttttgccattctcaccggattc
agtcgtcactcatggtgatttctcacttgataaccttatttttgacgaggggaaattaatagg
ttgtattgatgttggacgagtcggaatcgcagaccgataccaggatcttgccatcctatggaa
ctgcctcggtgagttttctccttcattacagaaacggctttttcaaaaatatggtattgataa
tcctgatatgaataaattgcagtttcatttgatgctcgatgagtttttctaatcagaattggt
taattggttgtaacactggcagagcattacgctgacttgacgggacggcggctttgttgaata
aatcgaacttttgctgagttgaaggatcagatcacgcatcttcccgacaacgcagaccgttcc
gtggcaaagcaaaagttcaaaatcaccaactggtccacctacaacaaagctctcatcaaccgt
ggctccctcactttctggctggatgatggggcgattcaggcctggtatgagtcagcaacacct
tcttcacgaggcagacctcagcgctcaaagatgcaggggtaaaagctaaccgcatctttaccg
acaaggcatccggcagttcaacagatcgggaagggctggatttgctgaggatgaaggtggagg
aaggtgatgtcattctggtgaagaagctcgaccgtcttggccgcgacaccgccgacatgatcc
aactgataaaagagtttgatgctcagggtgtagcggttcggtttattgacgacgggatcagta
ccgacggtgatatggggcaaatggtggtcaccaaggcctgctggtaatcaattgcctttttat
ttgggggagagggaagtcatgaaaaaactaacctttgaaattcgatctccagcacatcagcaa
aacgctattcacgcagtacagcaaatccttccagacccaaccaaaccaatcgtagtaaccatt
caggaacgcaaccgcagcttagaccaaaacaggaagctatgggcctgcttaggtgacgtctct
cgtcaggttgaatggcatggtcgctggctggatgcagaaagctggaagtgtgtgtttaccgca
gcattaaagcagcaggatgttgttcctaaccttgccgggaatggctttgtggtaataggccag
tcaaccagcaggatgcgtgtaggcgaatttgcggagctattagagcttatacaggcattcggt
acagagcgtggcgttaagtggtcagacgaagcgagactggctctggagtggaaagcgagatgg
ggagacagggctgcatgataaatgtcgttagtttctccggtggcaggacgtcagcatatttgc
tctggctaatggagcaaaagcgacgggcaggtaaagacgtgcattacgttttcatggatacag
gttgtgaacatccaatgacatatcggtttgtcagggaagttgtgaagttctgggatataccgc
tcaccgtattgcaggttgatatcaacccggagcttggacagccaaatggttatacggtatggg
aaccaaaggatattcagacgcgaatgcctgttctgaagccatttatcgatatggtaaagaaat
atggcactccatacgtcggcggcgcgttctgcactgacagattaaaactcgttcccttcacca
aatactgtgatgaccatttcgggcgagggaattacaccacgtggattggcatcagagctgatg
aaccgaagcggctaaagccaaagcctggaatcagatatcttgctgaactgtcagactttgaga
aggaagatatcctcgcatggtggaagcaacaaccattcgatttgcaaataccggaacatctcg
gtaactgcatattctgcattaaaaaatcaacgcaaaaaatcggacttgcctgcaaagatgagg
agggattgcagcgtgtttttaatgaggtcatcacgggatcccatgtgcgtgacggacatcggg
aaacgccaaaggagattatgtaccgaggaagaatgtcgctggacggtatcgcgaaaatgtatt
cagaaaatgattatcaagccctgtatcaggacatggtacgagctaaaagattcgataccggct
cttgttctgagtcatgcgaaatatttggagggcagcttgatttcgacttcgggagggaagctg
catgatgcgatgttatcggtgcggtgaatgcaaagaagataaccgcttccgaccaaatcaacc
ttactggaatcgatggtgtctccggtgtgaaagaacaccaacaggggtgttaccactaccgca
ggaaaaggaggacgtgtggcgagacagcgacgaagtatcaccgacataatctgcgaaaactgc
aaataccttccaacgaaacgcaccagaaataaacccaagccaatcccaaaagaatctgacgta
aaaaccttcaactacacggctcacctgtgggatatccggtggctaagacgtcgtgcgaggaaa
acaaggtgattgaccaaaatcgaagttacgaacaagaaagcgtcgagcgagctttaacgtgcg
ctaactgcggtcagaagctgcatgtgctggaagttcacgtgtgtgagcactgctgcgcagaac
tgatgagcgatccgaatagctcgatgcacgaggaagaagatgatggctaaaccagcgcgaaga
cgatgtaaaaacgatgaatgccgggaatggtttcaccctgcattcgctaatcagtggtggtgc
tctccagagtgtggaaccaagatagcactcgaacgacgaagtaaagaacgcgaaaaagcggaa
aaagcagcagagaagaaacgacgacgagaggagcagaaacagaaagataaacttaagattcga
aaactcgccttaaagccccgcagttactggattaaacaagcccaacaagccgtaaacgccttc
atcagagaaagagaccgcgacttaccatgtatctcgtgcggaacgctcacgtctgctcagtgg
gatgccggacattaccggacaactgctgcggcacctcaactccgatttaatgaacgcaatatt
cacaagcaatgcgtggtgtgcaaccagcacaaaagcggaaatctcgttccgtatcgcgtcgaa
ctgattagccgcatcgggcaggaagcagtagacgaaatcgaatcaaaccataaccgccatcgc
tggactatcgaagagtgcaaggcgatcaaggcagagtaccaacagaaactcaaagacctgcga
aatagcagaagtgaggccgcatgacgttctcagtaaaaaccattccagacatgctcgttgaag
catacggaaatcagacagaagtagcacgcagactgaaatgtagtcgcggtacggtcagaaaat
acgttgatgataaagacgggaaaatgcacgccatcgtcaacgacgttctcatggttcatcgcg
gatggagtgaaagagatgcgctattacgaaaaaattgatggcagcaaataccgaaatatttgg
gtagttggcgatctgcacggatgctacacgaacctgatgaacaaactggatacgattggattc
gacaacaaaaaagacctgcttatctcggtgggcgatttggttgatcgtggtgcagagaacgtt
gaatgcctggaattaatcacattcccctggttcagagctgtacgtggaaaccatgagcaaatg
atgattgatggcttatcagagcgtggaaacgttaatcactggctgcttaatggcggtggctgg
ttctttaatctcgattacgacaaagaaattctggctaaagctcttgcccataaagcagatgaa
cttccgttaatcatcgaactggtgagcaaagataaaaaatatgttatctgccacgccgattat
ccctttgacgaatacgagtttggaaagccagttgatcatcagcaggtaatctggaaccgcgaa
cgaatcagcaactcacaaaacgggatcgtgaaagaaatcaaaggcgcggacacgttcatcttt
ggtcatacgccagcagtgaaaccactcaagtttgccaaccaaatgtatatcgataccggcgca
gtgttctgcggaaacctaacattgattcaggtacagggagaaggcgcatgagactcgaaagcg
tagctaaatttcattcgccaaaaagcccgatgatgagcgactcaccacgggccacggcttctg
actctctttccggtactgatgtgatggctgctatggggatggcgcaatcacaagccggattcg
gtatggctgcattctgcggtaagcacgaactcagccagaacgacaaacaaaaggctatcaact
atctgatgcaatttgcacacaaggtatcggggaaataccgtggtgtggcaaagcttgaaggaa
atactaaggcaaaggtactgcaagtgctcgcaacattcgcttatgcggattattgccgtagtg
ccgcgacgccgggggcaagatgcagagattgccatggtacaggccgtgcggttgatattgcca
aaacagagctgtgggggagagttgtcgagaaagagtgcggaagatgcaaaggcgtcggctatt
caaggatgccagcaagcgcagcatatcgcgctgtgacgatgctaatcccaaaccttacccaac
ccacctggtcacgcactgttaagccgctgtatgacgctctggtggtgcaatgccacaaagaag
agtcaatcgcagacaacattttgaatgcggtcacacgttagcagcatgattgccacggatggc
aacatattaacggcatgatattgacttattgaataaaattgggtaaatttgactcaacgatgg
gttaattcgctcgttgtggtagtgagatgaaaagaggcggcgcttactaccgattccgcctag
ttggtcacttcgacgtatcgtctggaactccaaccatcgcaggcagagaggtctgcaaaatgc
aatcccgaaacagttcgcaggtaatagttagagcctgcataacggtttcgggattttttatat
ctgcacaacaggtaagagcattgagtcgataatcgtgaagagtcggcgagcctggttagccag
tgctctttccgttgtgctgaattaagcgaataccggaagcagaaccggatcaccaaatgcgta
caggcgtcatcgccgcccagcaacagcacaacccaaactgagccgtagccactgtctgtcctg
aattcattagtaatagttacgctgcggccttttacacatgaccttcgtgaaagcgggtggcag
gaggtcgcgctaacaacctcctgccgttttgcccgtgcatatcggtcacgaacaaatctgatt
actaaacacagtagcctggatttgttctatcagtaatcgaccttattcctaattaaatagagc
aaatccccttattgggggtaagacatgaagatgccagaaaaacatgacctgttggccgccatt
ctcgcggcaaaggaacaaggcatcggggcaatccttgcgtttgcaatggcgtaccttcgcggc
agatataatggcggtgcgtttacaaaaacagtaatcgacgcaacgatgtgcgccattatcgcc
tggttcattcgtgaccttctcgacttcgccggactaagtagcaatctcgcttatataacgagc
gtgtttatcggctacatcggtactgactcgattggttcgcttatcaaacgcttcgctgctaaa
aaagccggagtagaagatggtagaaatcaataatcaacgtaaggcgttcctcgatatgctggc
gtggtcggagggaactgataacggacgtcagaaaaccagaaatcatggttatgacgtcattgt
aggcggagagctatttactgattactccgatcaccctcgcaaacttgtcacgctaaacccaaa
actcaaatcaacaggcgccaattgctggtcaccatcctgtcggctgtggcacaggctgaacgc
cggaggatcaaaaggatctaggtgaagatcctttttgataatctcatgaccaaaatcccttaa
cgtgagttttcgttccactgagcgtcagaccccgtagaaaagatcaaaggatcttcttgagat
cctttttttctgcgcgtaatctgctgcttgcaaacaaaaaaaccaccgctaccagcggtggtt
tgtttgccggatcaagagctaccaactctttttccgaaggtaactggcttcagcagagcgcag
ataccaaatactgttcttctagtgtagccgtagttaggccaccacttcaagaactctgtagca
ccgcctacatacctcgctctgctaatcctgttaccagtggctgctgccagtggcgataagtcg
tgtcttaccgggttggactcaagacgatagttaccggataaggcgcagcggtcgggctgaacg
gggggttcgtgcacacagcccagcttggagcgaacgacctacaccgaactgagatacctacag
cgtgagctatgagaaagcgccacgcttcccgaagggagaaaggcggacaggtatccggtaagc
ggcagggtcggaacaggagagcgcacgagggagcttccagggggaaacgcctggtatctttat
agtcctgtcgggtttcgccacctctgacttgagcgtcgatttttgtgatgctcgtcagggggg
cggagcctatggaaaaacgccagcaacgcggcctttttacggttcctggccttttgctggcct
tttgctcacatgt
miR-155 huSOD1#2 + miR-E huSOD1#7
(plasmid sequence)
SEQ ID NO: 57
cctgcaggcagctgcgcgctcgctcgctcactgaggccgcccgggcgtcgggcgacctttggt
cgcccggcctcagtgagcgagcgagcgcgcagagagggagtggccaactccatcactaggggt
tcctgcggccggtcgacattgattattgactagttattaatagtaatcaattacggggtcatt
agttcatagcccatatatggagttccgcgttacataacttacggtaaatggcccgcctggctg
accgcccaacgacccccgcccattgacgtcaataatgacgtatgttcccatagtaacgccaat
agggactttccattgacgtcaatgggtggagtatttacggtaaactgcccacttggcagtaca
tcaagtgtatcatatgccaagtacgccccctattgacgtcaatgacggtaaatggcccgcctg
gcattatgcccagtacatgaccttatgggactttcctacttggcagtacatctacgtattagt
catcgctattaccatggtcgaggtgagccccacgttctgcttcactctccccatctccccccc
ctccccacccccaattttgtatttatttattttttaattattttgtgcagcgatgggggcggg
gggggggggggggcgcgcgccaggcggggggggcggggcgaggggggggcggggcgaggcgga
gaggtgcggcggcagccaatcagagcggcgcgctccgaaagtttccttttatggcgaggcggc
ggcggcggcggccctataaaaagcgaagcgcgcggggggggagtcgctgcgcgctgccttcgc
cccgtgccccgctccgccgccgcctcgcgccgcccgccccggctctgactgaccgcgttactc
ccacaggtgagcgggcgggacggcccttctcctccgggctgtaattagcgcttggtttaatga
cggcttgtttcttttctgtggctgcgtgaaagccttgaggggctccgggagggccctttgtgc
ggggggagcggctcggggggtgcgtgcgtgtgtgtgtgcgtggggagcgccgcgtgcggctcc
gcgctgcccggcggctgtgagcgctgcgggcgcggcgcggggctttgtgcgctccgcagtgtg
cgcgaggggagcgcggccgggggcggtgccccgcggtgcgcaaccccccctgcacccccctcc
ccgagttgctgagcacggcccggcttcgggtgcggggctccgtacggggcgtggcgcggggct
cgccgtgccgggggggggtggcggcaggtgggggtgccgggcggggggggccgcctcgggccg
gggagggctcgggggaggggcgcggcggcccccggagcgccggcggctgtcgaggcgcggcga
gccgcagccattgccttttatggtaatcgtgcgagagggcgcagggacttcctttgtcccaaa
tctgtgcggagccgaaatctgggaggcgccgccgcaccccctctagcgggcgcggggcgaagc
ggtgcggcgccggcaggaaggaaatgggcggggagggccttcgtgcgtcgccgcgccgccgtc
cccttctccctctccagcctcggggctgtccgcggggggacggctgccttcgggggggacggg
gcagggcggggttcggcttctggcgtgtgaccggcggctctagagcctctgctaaccatgttc
atgccttcttctttttcctacaggctagcggtaccctggaggcttgctgaaggctgtatgctg
ATTACTTTCCTTCTGCTCGAAgttttggccactgactgacTTCGAGCAAGGAAAGTAATcagg
acacaaggcctgttactagcactcacatggaacaaatggccggtacctgtttgaatgaggctt
cagtactttacagaatcgttgcctgcacatcttggaaacacttgctgggattacttcgacttc
ttaacccaacagaaggctcgagAAGGTATATTGCTGTTGACAGTGAGCGCAGCTGTAGAAATG
TATCCTGATAGTGAAGCCACAGATGTATCAGGATACATTTCTACAGCTATGCCTACTGCCTCG
GACTTCAAGGGGCTAgaattcgagcaattatcttgtttactaaaactgaataccttgctatct
ctttgatacatttttacaaagctgaattaaaatggtataaattaaatcactttgagctCAATC
AACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACTGACAATTCCGTGG
TGTTGTCGGGGAAATCATCGTCCTTTCCTTGGCTGCTCGCCTGTGTTGCCACCTGGATTCTGC
GCGGGACGTCCTTCTGCTACGTCCCTTCGGCCCTCAATCCAGCGGACCTTCCTTCCCGCGGCC
TGCTGCCGGCTCTGCGGCCTCTTCCGCGTCTTCGCCTTCGCCCTCAGACGAGTCGGATCTCCC
TTTGGGCCGCCTCCCCGCTGATCACGCCTAGGACGGGTGGCATCCCTGTGACCCCTCCCCAGT
GCCTCTCCTGGCCCTGGAAGTTGCCACTCCAGTGCCCACCAGCCTTGTCCTAATAAAATTAAG
TTGCATCATTTTGTCTGACTAGGTGTCCTTCTATAATATTATGGGGTGGAGGGGGGTGGTATG
GAGCAAGGGGCAAGTTGGGAAGACAACCTGTAGGGCCTGCGGGGTCTATTGGGAACCAAGCTG
GAGTGCAGTGGCACAATCTTGGCTCACTGCAATCTCCGCCTCCTGGGTTCAAGCGATTCTCCT
GCCTCAGCCTCCCGAGTTGTTGGGATTCCAGGCATGCATGACCAGGCTCAGCTAATTTTTGTT
TTTTTGGTAGAGACGGGGTTTCACCATATTGGCCAGGCTGGTCTCCAACTCCTAATCTCAGGT
GATCTACCCACCTTGGCCTCCCAAATTGCTGGGATTACAGGCGTGAACCACTGCTCCCTTCCC
TGTCCTTActagtcggccgcaggaacccctagtgatggagttggccactccctctctgcgcgc
tcgctcgctcactgaggccgggcgaccaaaggtcgcccgacgcccgggctttgcccgggcggc
ctcagtgagcgagcgagcgcgcagctgcctgcaggggcgcctgatgcggtattttctccttac
gcatctgtgcggtatttcacaccgcatacgtcaaagcaaccatagtacgcgccctgtagcggc
gcattaagcgcggcgggtgtggtggttacgcgcagcgtgaccgctacacttgccagcgcctta
gcgcccgctcctttcgctttcttcccttcctttctcgccacgttcgccggctttccccgtcaa
gctctaaatcgggggctccctttagggttccgatttagtgctttacggcacctcgaccccaaa
aaacttgatttgggtgatggttcacgtagtgggccatcgccctgatagacggtttttcgccct
ttgacgttggagtccacgttctttaatagtggactcttgttccaaactggaacaacactcaac
cctatctcggtctattcttttgatttataagggattttgccgatttcggcctattggttaaaa
aatgagctgatttaacaaaaatttaacgcgaattttaacaaaatattaacgcttacaatttag
gtggcacttttcggggaaatgtgcgcggaacccctatttgtttatttttctaaatacattcaa
atatgtatccgctcatgagacaataaccctgataaatgcttcaataatattgaaaaaggaaga
gtatgagtattcaacatttccgtgtcgcccttattcccttttttgcggcattttgccttcctg
tttttgctcacccagaaacgctggtgaaagtaaaagatgctgaagatcagttgggtgcagtgt
ctcaaaatctctgatgttacattgcacaagataaaaatatatcatcatgaacaataaaactgt
ctgcttacataaacagtaatacaaggggtgttatgagccatattcaacgggaaacgtcttgct
cgaggccgcgattaaattccaacatggatgctgatttatatgggtataaatgggctcgcgata
atgtcgggcaatcaggtgcgacaatctatcgattgtatgggaagcccgatgcgccagagttgt
ttctgaaacatggcaaaggtagcgttgccaatgatgttacagatgagatggtcagactaaact
ggctgacggaatttatgcctcttccgaccatcaagcattttatccgtactcctgatgatgcat
ggttactcaccactgcgatccccgggaaaacagcattccaggtattagaagaatatcctgatt
caggtgaaaatattgttgatgcgctggcagtgttcctgcgccggttgcattcgattcctgttt
gtaattgtccttttaacagcgatcgcgtatttcgtctcgctcaggcgcaatcacgaatgaata
acggtttggttgatgcgagtgattttgatgacgagcgtaatggctggcctgttgaacaagtct
ggaaagaaatgcataagcttttgccattctcaccggattcagtcgtcactcatggtgatttct
cacttgataaccttatttttgacgaggggaaattaataggttgtattgatgttggacgagtcg
gaatcgcagaccgataccaggatcttgccatcctatggaactgcctcggtgagttttctcctt
cattacagaaacggctttttcaaaaatatggtattgataatcctgatatgaataaattgcagt
ttcatttgatgctcgatgagtttttctaatcagaattggttaattggttgtaacactggcaga
gcattacgctgacttgacgggacggcggctttgttgaataaatcgaacttttgctgagttgaa
ggatcagatcacgcatcttcccgacaacgcagaccgttccgtggcaaagcaaaagttcaaaat
caccaactggtccacctacaacaaagctctcatcaaccgtggctccctcactttctggctgga
tgatggggcgattcaggcctggtatgagtcagcaacaccttcttcacgaggcagacctcagcg
ctcaaagatgcaggggtaaaagctaaccgcatctttaccgacaaggcatccggcagttcaaca
gatcgggaagggctggatttgctgaggatgaaggtggaggaaggtgatgtcattctggtgaag
aagctcgaccgtcttggccgcgacaccgccgacatgatccaactgataaaagagtttgatgct
cagggtgtagcggttcggtttattgacgacgggatcagtaccgacggtgatatggggcaaatg
gtggtcaccaaggcctgctggtaatcaattgcctttttatttgggggagagggaagtcatgaa
aaaactaacctttgaaattcgatctccagcacatcagcaaaacgctattcacgcagtacagca
aatccttccagacccaaccaaaccaatcgtagtaaccattcaggaacgcaaccgcagcttaga
ccaaaacaggaagctatgggcctgcttaggtgacgtctctcgtcaggttgaatggcatggtcg
ctggctggatgcagaaagctggaagtgtgtgtttaccgcagcattaaagcagcaggatgttgt
tcctaaccttgccgggaatggctttgtggtaataggccagtcaaccagcaggatgcgtgtagg
cgaatttgcggagctattagagcttatacaggcattcggtacagagcgtggcgttaagtggtc
agacgaagcgagactggctctggagtggaaagcgagatggggagacagggctgcatgataaat
gtcgttagtttctccggtggcaggacgtcagcatatttgctctggctaatggagcaaaagcga
cgggcaggtaaagacgtgcattacgttttcatggatacaggttgtgaacatccaatgacatat
cggtttgtcagggaagttgtgaagttctgggatataccgctcaccgtattgcaggttgatatc
aacccggagcttggacagccaaatggttatacggtatgggaaccaaaggatattcagacgcga
atgcctgttctgaagccatttatcgatatggtaaagaaatatggcactccatacgtcggcggc
gcgttctgcactgacagattaaaactcgttcccttcaccaaatactgtgatgaccatttcggg
cgagggaattacaccacgtggattggcatcagagctgatgaaccgaagcggctaaagccaaag
cctggaatcagatatcttgctgaactgtcagactttgagaaggaagatatcctcgcatggtgg
aagcaacaaccattcgatttgcaaataccggaacatctcggtaactgcatattctgcattaaa
aaatcaacgcaaaaaatcggacttgcctgcaaagatgaggagggattgcagcgtgtttttaat
gaggtcatcacgggatcccatgtgcgtgacggacatcgggaaacgccaaaggagattatgtac
cgaggaagaatgtcgctggacggtatcgcgaaaatgtattcagaaaatgattatcaagccctg
tatcaggacatggtacgagctaaaagattcgataccggctcttgttctgagtcatgcgaaata
tttggagggcagcttgatttcgacttcgggagggaagctgcatgatgcgatgttatcggtgcg
gtgaatgcaaagaagataaccgcttccgaccaaatcaaccttactggaatcgatggtgtctcc
ggtgtgaaagaacaccaacaggggtgttaccactaccgcaggaaaaggaggacgtgtggcgag
acagcgacgaagtatcaccgacataatctgcgaaaactgcaaataccttccaacgaaacgcac
cagaaataaacccaagccaatcccaaaagaatctgacgtaaaaaccttcaactacacggctca
cctgtgggatatccggtggctaagacgtcgtgcgaggaaaacaaggtgattgaccaaaatcga
agttacgaacaagaaagcgtcgagcgagctttaacgtgcgctaactgcggtcagaagctgcat
gtgctggaagttcacgtgtgtgagcactgctgcgcagaactgatgagcgatccgaatagctcg
atgcacgaggaagaagatgatggctaaaccagcgcgaagacgatgtaaaaacgatgaatgccg
ggaatggtttcaccctgcattcgctaatcagtggtggtgctctccagagtgtggaaccaagat
agcactcgaacgacgaagtaaagaacgcgaaaaagcggaaaaagcagcagagaagaaacgacg
acgagaggagcagaaacagaaagataaacttaagattcgaaaactcgccttaaagccccgcag
ttactggattaaacaagcccaacaagccgtaaacgccttcatcagagaaagagaccgcgactt
accatgtatctcgtgcggaacgctcacgtctgctcagtgggatgccggacattaccggacaac
tgctgcggcacctcaactccgatttaatgaacgcaatattcacaagcaatgcgtggtgtgcaa
ccagcacaaaagcggaaatctcgttccgtatcgcgtcgaactgattagccgcatcgggcagga
agcagtagacgaaatcgaatcaaaccataaccgccatcgctggactatcgaagagtgcaaggc
gatcaaggcagagtaccaacagaaactcaaagacctgcgaaatagcagaagtgaggccgcatg
acgttctcagtaaaaaccattccagacatgctcgttgaagcatacggaaatcagacagaagta
gcacgcagactgaaatgtagtcgcggtacggtcagaaaatacgttgatgataaagacgggaaa
atgcacgccatcgtcaacgacgttctcatggttcatcgcggatggagtgaaagagatgcgcta
ttacgaaaaaattgatggcagcaaataccgaaatatttgggtagttggcgatctgcacggatg
ctacacgaacctgatgaacaaactggatacgattggattcgacaacaaaaaagacctgcttat
ctcggtgggogatttggttgatcgtggtgcagagaacgttgaatgcctggaattaatcacatt
cccctggttcagagctgtacgtggaaaccatgagcaaatgatgattgatggcttatcagagcg
tggaaacgttaatcactggctgcttaatggcggtggctggttctttaatctcgattacgacaa
agaaattctggctaaagctcttgcccataaagcagatgaacttccgttaatcatcgaactggt
gagcaaagataaaaaatatgttatctgccacgccgattatccctttgacgaatacgagtttgg
aaagccagttgatcatcagcaggtaatctggaaccgcgaacgaatcagcaactcacaaaacgg
gatcgtgaaagaaatcaaaggcgcggacacgttcatctttggtcatacgccagcagtgaaacc
actcaagtttgccaaccaaatgtatatcgataccggcgcagtgttctgcggaaacctaacatt
gattcaggtacagggagaaggcgcatgagactcgaaagcgtagctaaatttcattcgccaaaa
agcccgatgatgagcgactcaccacgggccacggcttctgactctctttccggtactgatgtg
atggctgctatggggatggcgcaatcacaagccggattcggtatggctgcattctgcggtaag
cacgaactcagccagaacgacaaacaaaaggctatcaactatctgatgcaatttgcacacaag
gtatcggggaaataccgtggtgtggcaaagcttgaaggaaatactaaggcaaaggtactgcaa
gtgctcgcaacattcgcttatgcggattattgccgtagtgccgcgacgccgggggcaagatgc
agagattgccatggtacaggccgtgcggttgatattgccaaaacagagctgtgggggagagtt
gtcgagaaagagtgcggaagatgcaaaggcgtcggctattcaaggatgccagcaagcgcagca
tatcgcgctgtgacgatgctaatcccaaaccttacccaacccacctggtcacgcactgttaag
ccgctgtatgacgctctggtggtgcaatgccacaaagaagagtcaatcgcagacaacattttg
aatgcggtcacacgttagcagcatgattgccacggatggcaacatattaacggcatgatattg
acttattgaataaaattgggtaaatttgactcaacgatgggttaattcgctcgttgtggtagt
gagatgaaaagaggcggcgcttactaccgattccgcctagttggtcacttcgacgtatcgtct
ggaactccaaccatcgcaggcagagaggtctgcaaaatgcaatcccgaaacagttcgcaggta
atagttagagcctgcataacggtttcgggattttttatatctgcacaacaggtaagagcattg
agtcgataatcgtgaagagtcggcgagcctggttagccagtgctctttccgttgtgctgaatt
aagcgaataccggaagcagaaccggatcaccaaatgcgtacaggcgtcatcgccgcccagcaa
cagcacaacccaaactgagccgtagccactgtctgtcctgaattcattagtaatagttacgct
gcggccttttacacatgaccttcgtgaaagcgggtggcaggaggtcgcgctaacaacctcctg
ccgttttgcccgtgcatatcggtcacgaacaaatctgattactaaacacagtagcctggattt
gttctatcagtaatcgaccttattcctaattaaatagagcaaatccccttattgggggtaaga
catgaagatgccagaaaaacatgacctgttggccgccattctcgcggcaaaggaacaaggcat
cggggcaatccttgcgtttgcaatggcgtaccttcgcggcagatataatggcggtgcgtttac
aaaaacagtaatcgacgcaacgatgtgcgccattatcgcctggttcattcgtgaccttctcga
cttcgccggactaagtagcaatctcgcttatataacgagcgtgtttatcggctacatcggtac
tgactcgattggttcgcttatcaaacgcttcgctgctaaaaaagccggagtagaagatggtag
aaatcaataatcaacgtaaggcgttcctcgatatgctggcgtggtcggagggaactgataacg
gacgtcagaaaaccagaaatcatggttatgacgtcattgtaggcggagagctatttactgatt
actccgatcaccctcgcaaacttgtcacgctaaacccaaaactcaaatcaacaggcgccaatt
gctggtcaccatcctgtcggctgtggcacaggctgaacgccggaggatcaaaaggatctaggt
gaagatcctttttgataatctcatgaccaaaatcccttaacgtgagttttcgttccactgagc
gtcagaccccgtagaaaagatcaaaggatcttcttgagatcctttttttctgcgcgtaatctg
ctgcttgcaaacaaaaaaaccaccgctaccagcggtggtttgtttgccggatcaagagctacc
aactctttttccgaaggtaactggcttcagcagagcgcagataccaaatactgttcttctagt
gtagccgtagttaggccaccacttcaagaactctgtagcaccgcctacatacctcgctctgct
aatcctgttaccagtggctgctgccagtggcgataagtcgtgtcttaccgggttggactcaag
acgatagttaccggataaggcgcagcggtcgggctgaacggggggttcgtgcacacagcccag
cttggagcgaacgacctacaccgaactgagatacctacagcgtgagctatgagaaagcgccac
gcttcccgaagggagaaaggcggacaggtatccggtaagcggcagggtcggaacaggagagcg
cacgagggagcttccagggggaaacgcctggtatctttatagtcctgtcgggtttcgccacct
ctgacttgagcgtcgatttttgtgatgctcgtcaggggggcggagcctatggaaaaacgccag
caacgcggcctttttacggttcctggccttttgctggccttttgctcacatgt
Bovine Growth Hormone (bGH) polyA sequence
(5′ to 3′ on either the plus strand or
the minus strand)
SEQ ID NO: 58
ctgtgccttctagttgccagccatctgttgtttgcccctcccccgtgccttccttgaccctgg
aaggtgccactcccactgtcctttcctaataaaatgaggaaattgcatcgcattgtctgagta
ggtgtcattctattctggggggtggggtggtgcaggacagcaagggggaggattgggaagaca
atagcaggcatgctggggatgcggtgggctctatgg
SV40 polyA sequence (5′ to 3′ on either the
plus strand or the minus strand)
SEQ ID NO: 59
AACTTGTTTATTGCAGCTTATAATGGTTACAAATAAAGCAATAGCATCACAAATTTCACAAAT
AAAGCATTTTTTTCACTGCATTCTAGTTGTGGTTTGTCCAAACTCATCAATGTATCTTATCAT
GTCTGGATC
rb-Glob polyA sequence (5′ to 3′ on either the
plus strand or the minus strand)
SEQ ID NO: 60
attcactcctcaggtgcaggctgcctatcagaaggtggtggctggtgtggccaatgccctggc
tcacaaataccactgagatctttttccctctgccaaaaattatggggacatcatgaagcccct
tgagcatctgacttctggctaataaaggaaatttattttcattgcaatagtgtgttggaattt
tttgtgtctctcactcggaaggacatatgggagggcaaatcatttaaaacatcagaatgagta
tttggtttagagtttggcaacatatgcccatatgctggctgccatgaacaaaggttggctata
aagaggtcatcagtatatgaaacagccccctgctgtccattccttattccatagaaaagcctt
gacttgaggttagattttttttatattttgttttgtgttatttttttctttaacatccctaaa
attttccttacatgttttactagccagatttttcctcctctcctgactactcccagtcatagc
tgtccctcttctcttatggagatccctcgacctgcagcccaagcttggcgtaa
beta-Glob polyA sequence (5′ to 3′ on either the
plus strand or the minus strand)
SEQ ID NO: 61
GCTCGCTTTCTTGCTGTCCAATTTCTATTAAAGGTTCCTTTGTTCCCTAAGTCCAACTACTAA
ACTGGGGGATATTATGAAGGGCCTTGAGCATCTGGATTCTGCCTAATAAAAAACATTTATTTT
CATTGCAATGATGTATTTAAATTATTTCTGAATATTTTACTAAAAAGGGAATGTGGGAGGTCA
GTGCATTTAAAACATAAAGAAATGAAGAGCTAGTTCAAACCTTGGGAAAATACACTATATCTT
AAACTCCATGAAAGAAGGTGAGGCTGCAAACAGCTAATGCACATTGGCAACAGCCCCTGATGC
CTATGCCTTATTCATCCCTCAGAAAAGGATTCAAGTAGAGGCTTGATTTGGAGGTTAAAGTTT
TGCTATGCTGTATTTTA
Synthetic polyA sequence (5′ to 3′ on either the
plus strand or the minus strand)
SEQ ID NO: 62
AATAAAATATCTTTATTTTCATTACATCTGTGTGTTGGTTTTTTGTGTG
HSV TK polyA sequence (5′ to 3′ on either the
plus strand or the minus strand)
SEQ ID NO: 63
cggcaataaaaagacagaataaaacgcacgggtgttgggtcgtttgttca
Synthetic polyA sequence + transcription pause site
(5′ to 3′ on either the plus strand or the minus strand)
SEQ ID NO: 64
AATAAAATATCTTTATTTTCATTACATCTGTGTGTTGGTTTTTTGTGTGaacatacgctctcc
atcaaaacaaaacgaaacaaaacaaactagcaaaataggctgtccccagtgcaagtgcaggtg
ccagaacatttctct

### EXAMPLES

The following Examples are provided for illustration and are not in any way to limit the scope of the disclosure. One of skill in the art will appreciate that certain design and selection criteria as described herein may be changed according to common practices in the field.

### Example 1: Selection of Antisense Oligonucleotide Sequences that Target SOD1 and Design of miR-SOD1 Vectors

The human SOD1 gene on chromosome 21 is 9310 bp in length and transcribes a mature mRNA of 980 nt that encodes a protein product of 154 amino acids. Twelve shRNAs were designed by Mirimus Inc. and Transomic Technologies based on two published algorithms for complementarity to the human SOD1 mRNA and pre-mRNA (NM_00454.4) (see Table 1 for shRNA sequences). The algorithms predicted these shRNAs to be potent in mediating RNAi and unable to hybridize to any other known human mRNA (Auyeung et al. (2013); Pelossof et al. (2017)). Alignment of the twelve shRNA candidates to the SOD1 mRNA from Mus musculus, Macaca fascicularis, Callithrix jacchus or Macaca mulatta was also performed to determine potential cross-species reactivity.

The twelve shRNAs described above were embedded in murine miR-155 scaffold (SEQ ID NO: 16) flanking sequences according to Invitrogen Block-iT RNAi Designer kit manual to form the candidates miR-155-SOD1-#1 to miR-155-SOD1-#12, each of which were cloned into a mammalian expressing vector containing a CASI promoter, Emerald Green Fluorescent Protein, woodchuck hepatitis virus post-transcriptional regulatory element (WPRE), and bovine growth hormone polyadenylation signal (bGH polyA) signal (SEQ ID NO: 15).

TABLE 1

| | | | | mismatches |
|---|---|---|---|---|
| | | | mismatches | with |
| | | guide SEQ | with | Callithrix |
| guide name | guide sequence | ID NO | Mus musculus | jacchus |
| shRNA-huSODI_#1 | TCTGCTCGAAATTGATGATGC | huSOD1#1 | 3 | 0 |
| shRNA-buSOD1_#2 | ATTACTTTCCTTCTGCTCGAA | huSOD1#2 | 4 | 0 |
| shRNA-bwSOD1_#3 | ATGAACATGGAATCCATGCAG | huSOD1#3 | 4 | 0 |
| shRNA-BUSOD1_#4 | TTCAATAGACACATCGGCCAC | huSOD1#4 | 2 | 3 |
| shRNA-huSOD1_#5 | TACTTTCTTCATTTCCACCTT | huSOD1#5 | 0 | 0 |
| shRNA-huSOD1_#6 | TTTGTACTTTCTTCATTTCCA | huSOD1#5 | 0 | 0 |
| shRNA-huSOD1_#7 | TCAGGATACATTTCTACAGCT | huSOD1#7 | no match | 2 |
| shRNA-huSOD1_#8 | TTATCAGGATACATTTCTACA | huSOD1#8 | no match | 1 |
| sKRNA-huSOD1_#9 | TTACAGTGTTTAATGTTTATC | huSOD1#9 | 5 | 1 |
| shRNA-huSOD1_#10 | TACACTTTTAAGATTACAGTG | huSOD1#10 | 8 | 2 |
| shRNA-huSOD1_#11 | AATGACAAAGAAATTCTGACA | huSOD1#11 | no match | 16 |
| shRNA-huSOD1_#12 | TTTAGTTTGAATTTGGATTCT | huSOD1#12 | no match | no match |

### Example 2: In Vitro Screening of miR-SOD1 Vectors

To test efficacy of selected shRNAs, cell lines were transfected with the miR-155-SOD1 vectors described above, along with another vector encoding human SOD1, including 5′UTR, open reading frame, and 3′UTR. Four cell lines were used including HEK293T, HeLa, COS1, and Neuro2A. Protein knockdown levels were quantified by immunoblotting and quantified by LI-COR imaging system (LI-COR Biosciences) at either 24 hours or 48 hours after a-miR delivery by FuGENE HD transfection (Promega) (Table 2).

TABLE 2

| | ranking (low is good) | | | | | |
|---|---|---|---|---|---|---|
| p155-miR155 | Neuro2A | COS1 | HeLa (48 h) | HeLa (24 h) | HEK293T (24 h) | avg rank |
| miR155-huSOD1_#01 | 8 | 7 | 7 | 11 | 9 | 7.8 |
| miR155-huSOD1_#02 [symbol] | 1 | 3 | 1 | 1 | 2 | 1.8 |
| miR155-huSOD1_#03 [symbol] | 3 | 2 | 6 | 4 | 5 | 4.0 |
| miR155-huSOD1_#04 | 9 | 9 | 8 | [symbol] | 4 | 7.5 |
| miR155-huSOD1_#05 [symbol] | 7 | 8 | [symbol] | [symbol] | [symbol] | [symbol] |
| miR155-huSOD1_#06 | 4 | 5 | [symbol] | 6 | 11 | 7.3 |
| miR155-huSOD1_#07 [symbol] | 2 | 1 | 2 | 3 | 3 | 2.0 |
| miR155-huSOD1_#08 [symbol] | 5 | 4 | 3 | 5 | 1 | 3.3 |
| miR155-huSOD1_#09 [symbol] | [symbol] | [symbol] | [symbol] | [symbol] | [symbol] | [symbol] |
| miR155-huSOD1_#10 | 10 | 10 | 10 | 10 | [symbol] | 9.3 |
| miR155-huSOD1_#11 | 11 | 11 | 12 | 9 | 12 | 11.5 |
| miR155-huSOD1_#12 | 12 | 12 | 11 | 12 | 10 | 11.3 |
| | Avg % (huSOD1/GAPDH) remaining | | | | | |
| p155-miR155 | Neuro2A | COS1 | HeLa (48 h) | HeLa (24 h) | HEK293T (24 h) | avg remain |
| miR155-huSOD1_#01 | 31.1 | 5 [symbol] .7 | 50.3 | 92.6 | 8 [symbol] .9 | 56.3 |
| miR155-huSOD1_#02 [symbol] | 2.6 | 29.7 | [symbol] | 66.4 | 62.8 | 32.2 |
| miR155-huSOD1_#03 [symbol] | 9.7 | 28.2 | 48.5 | 74.9 | 73.5 | 40.0 |
| miR155-huSOD1_#04 | 38.0 | 75.1 | 52.8 | 79.1 | 7 [symbol] .3 | 59.8 |
| miR155-huSOD1_#05 [symbol] | [symbol] | [symbol] | [symbol] | [symbol] | [symbol] | [symbol] |
| miR155-huSOD1_#06 | 14.0 | 39.2 | 64.7 | 78. [symbol] | 92.9 | 52.7 |
| miR155-huSOD1_#07 [symbol] | 8.0 | 17.3 | 34.7 | 74.7 | 70.4 | 32.6 |
| miR155-huSOD1_#08 [symbol] | 14.9 | 33.9 | 42.7 | 75.5 | 60.7 | 38.0 |
| miR155-huSOD1_#09 [symbol] | [symbol] | [symbol] | [symbol] | [symbol] | [symbol] | [symbol] |
| miR155-huSOD1_#10 | 39.0 | 75.9 | 70.6 | 85.8 | 80.7 | 66.5 |
| miR155-huSOD1_#11 | 61.2 | 89.6 | 89.3 | 85.7 | 94.7 | 83.7 |
| miR155-huSOD1_#12 | 89.0 | 106.0 | 86.8 | 110. [symbol] | 90.8 | 93.2 |

[symbol]

indicates data missing or illegible when filed

### Example 3: In Vitro Screening of miR-SOD1 Vectors in Different miRNA Scaffolds

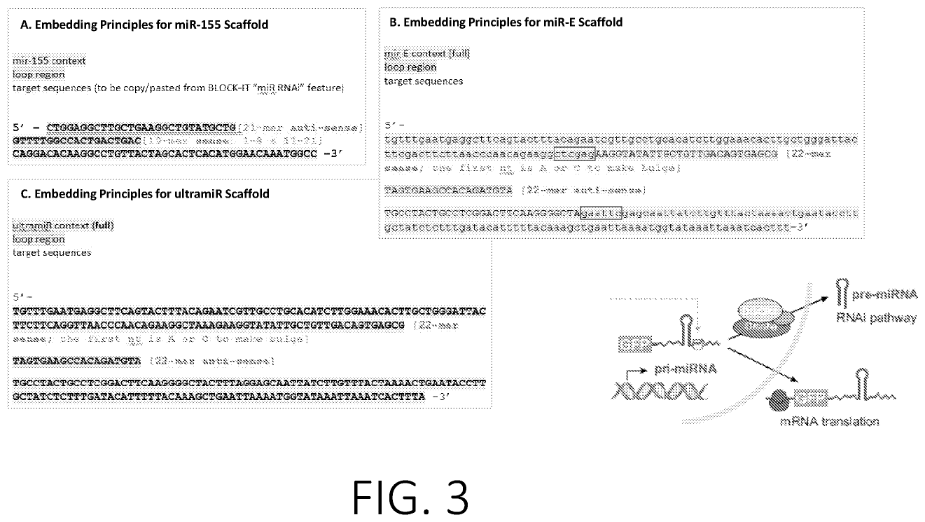

In addition to the miR-155 scaffold (SEQ ID NO: 16), the six most potent shRNAs described in Example 2 were subsequently embedded in miR-E scaffolds (SEQ ID NO:17), or ultramiR scaffolds (SEQ ID NO: 18), yielding 18 a-miR lead candidates (SEQ ID NOs: 22-39) [Fellmann et al. (2013); Fowler et al. (2016)]. The miR-E scaffold sequence was provided by Mirimus Inc. and the ultramiR scaffold sequence was provided by Transomic Technologies. Embedding principles were illustrated in FIGS. 3A-3C.

These 18 a-miR lead candidates were each individually cloned into a single-stranded AAV9 vector that consists of inverted terminal repeats (ITRs), CAG promoter (SEQ ID NO: 40), mCherry (SEQ ID NO: 54), WPRE (SEQ ID NO: 44), and bovine growth hormone polyadenylation signal (bGH polyA). A second set was cloned into a single-stranded AAV9 vector that consists of inverted terminal repeats (ITRs), CAG promoter (SEQ ID NO: 40), WPRE (SEQ ID NO: 44), and human growth hormone polyadenylation signal (hGH polyA) (SEQ ID NO: 45).

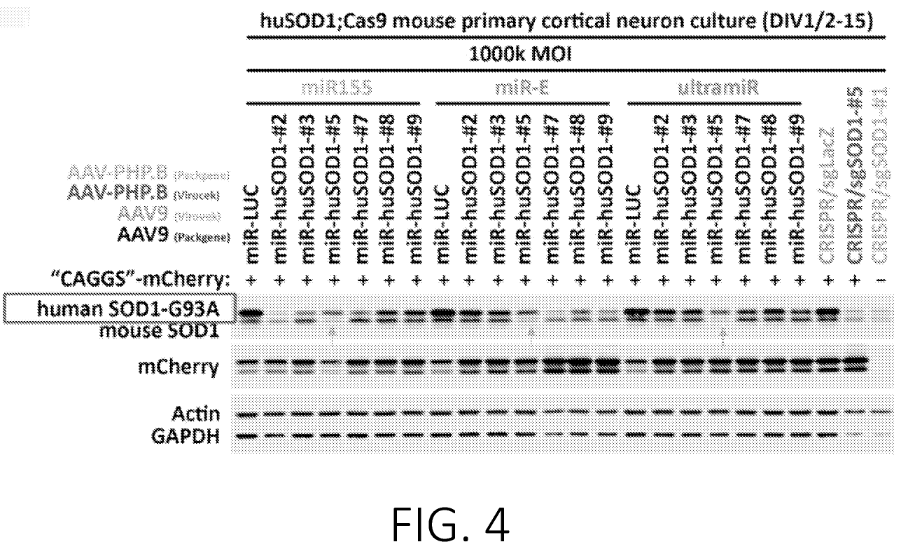

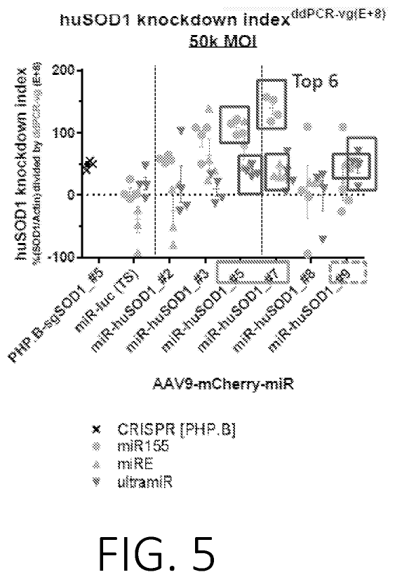

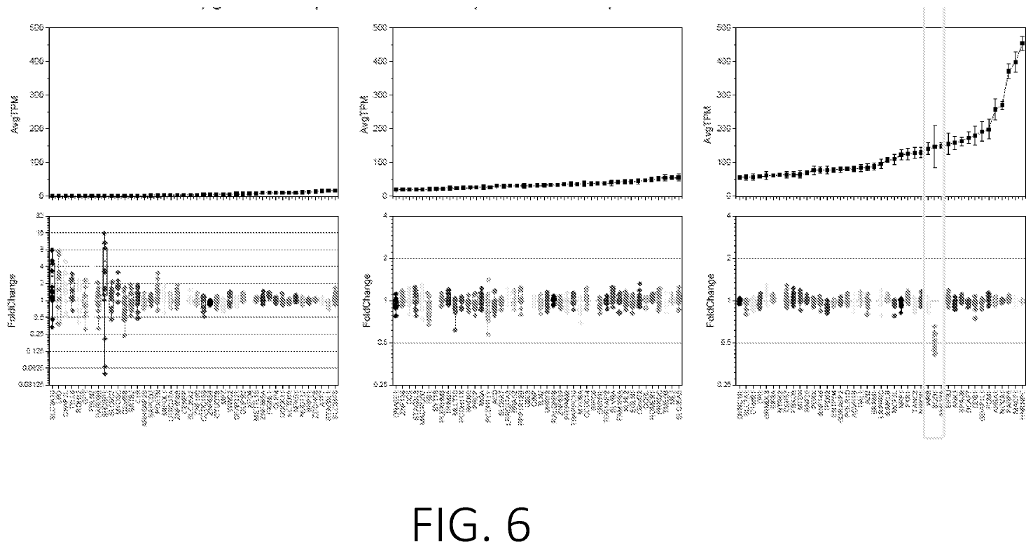

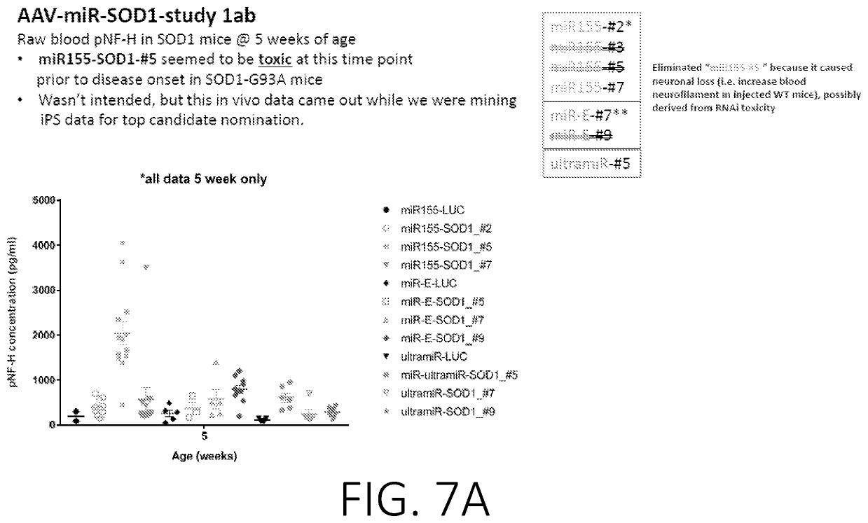

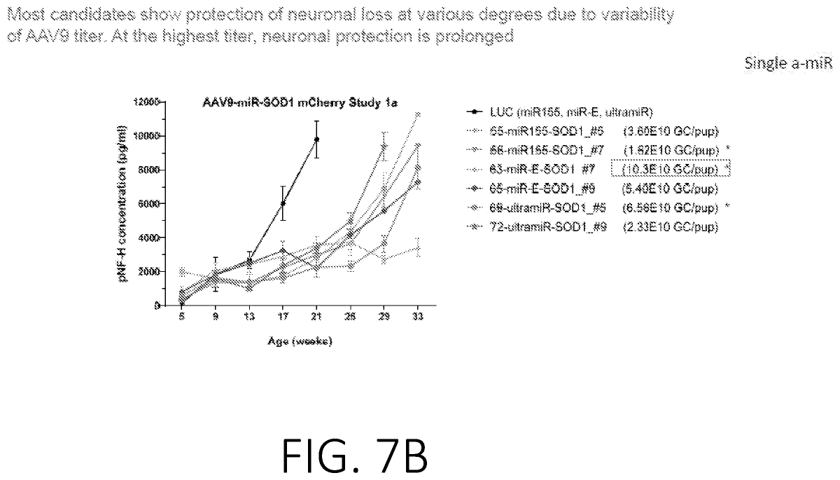

The SOD1-knockdown efficiency of these 18 candidate a-miRs were evaluated by transducing primary cortical neuron culture prepared from transgenic mice expressing the human SOD1-G93A at multiplicity of infection (MOI) of 50k, 250k and 1000k. Protein analysis was conducted by immunoblotting roughly 2 weeks after AAV transduction (FIG. 4). Seven a-miR candidates among the original 18 candidates, including miR-155-SOD1-#2, miR-155-SOD1-#3, miR-155-SOD1-#5, miR-155-SOD1-#7, miR-E-SOD1-#7, miR-E-SOD1-#9 and ultramiR-SOD1-#5, inhibited human SOD1 protein expression by greater than 50% at MOI of 250k, and therefore were selected for further development (Data not shown).

Next, NGS technology of 75 base-long single-end read miRNA-seq was used at a depth of 10 million reads per sample to analyze the a-miR processing profiles of these lead candidates. The goal is to identify and then eliminate any potential off-target risks from the expression and processing of a-miRs. Infidelity in a-miR processing leads to expression of unintended guide sequences which could potentially bind to other mRNAs in the transcriptome. The 7 a-miR candidates were further examined for a-miR processing properties, including sequence accuracy of guide strands, production level of guide strands, and guide strand to passenger strand expression ratios. The passenger strand is a “by-product” of the a-miR processing pathway. It harbors sequences homologous to target mRNA and is often found to be more labile than the guide strand. Human iPS-derived Neurogenin 2 (NGN2) excitatory cortical neurons and human HeLa cells were transduced with AAV9 that encodes each of the seven a-miR candidates. Analysis of a-miR guide strands and passenger strands was conducted by small RNAseq. Inhibition of endogenous human SOD1 mRNA was assessed by RT-qPCR. Based on correlation analysis between steady-state levels of guide strand and its ability to inhibit endogenous human SOD1 in iPS-NGN2 cells, miR-155-SOD1-#3 was discarded from further development due to the highest production level of its guide strand and low inhibition efficiency of huSOD1. Based on comparison between steady-state levels of effective guide strand and futile passenger strand, miR-E-SOD1-#9 was discarded from further development due to its having the lowest ratio of guide strand to passenger strand in both iPS-NGN2 cells and HeLa cell line (data not shown).

To further examine production level of the guide strands in vivo, AAV9 encoding a-miR-SOD1 candidates were administered in wild-type C57BL6/J mice with a single ICV bolus injection on postnatal day 0 (P0). CNS tissues were collected 10 weeks following injection. Analysis of a-miR guide strands was conducted by small RNAseq. AAV9 viral genomes (vg) distributed to CNS tissues were quantified by qPCR. In the scatter plot of correlation between steady-state level of guide strand and AAV9 viral genome copy (GC), the steepest slope of the regression line was observed for miR-155-SOD1-#5 and indicated the possibility of excessive guide strand production upon a small increment of AAV dose (data not shown). Due to concerns about potential RNAi stress associated with over production of a-miR if a higher AAV dose is administered, miR-155-SOD1-#5 was discarded from further therapeutic development but was kept in studies conducted to model RNAi stress in vivo.

The a-miR lead candidates were designed such that they would not hybridize to any other known human gene. The sequences of both guide strand and passenger strand of the top four a-miR candidates were searched in silico against the human transcriptome for end-to-end alignment. SOD1 was the only human RNA transcript identified with zero mismatch. To determine if another human RNA transcript with more than a single-base mismatch could potentially be bound by the a-miR candidates in neurons, human iPS-derived NGN2 excitatory cortical neuron culture was transduced with AAV9 encoding miR-155-SOD1-#2, miR-155-SOD1-#7, miR-E-SOD1-#7, or ultramiR-SOD1-#5 respectively. Differentially expressed genes were analyzed by bulk mRNAseq with a coverage of approximately 20 reads per base. The results showed that only SOD1 was significantly downregulated in the iPS-NGN2 neurons treated with a-miR-SOD1 candidates.

### Example 4: In Vivo Testing of AAV-miR-SOD1

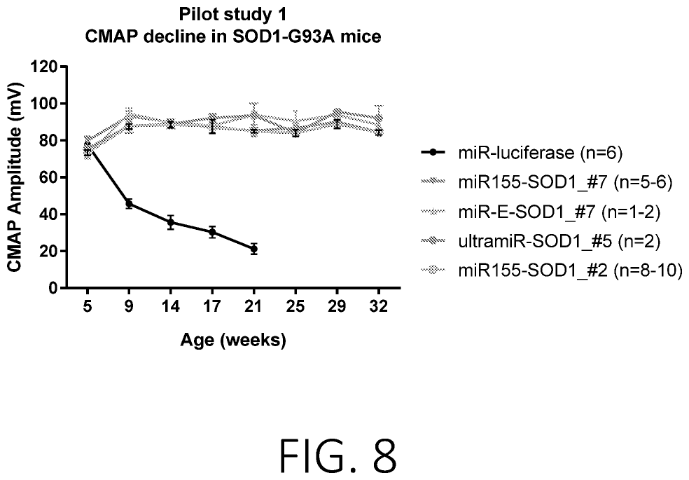

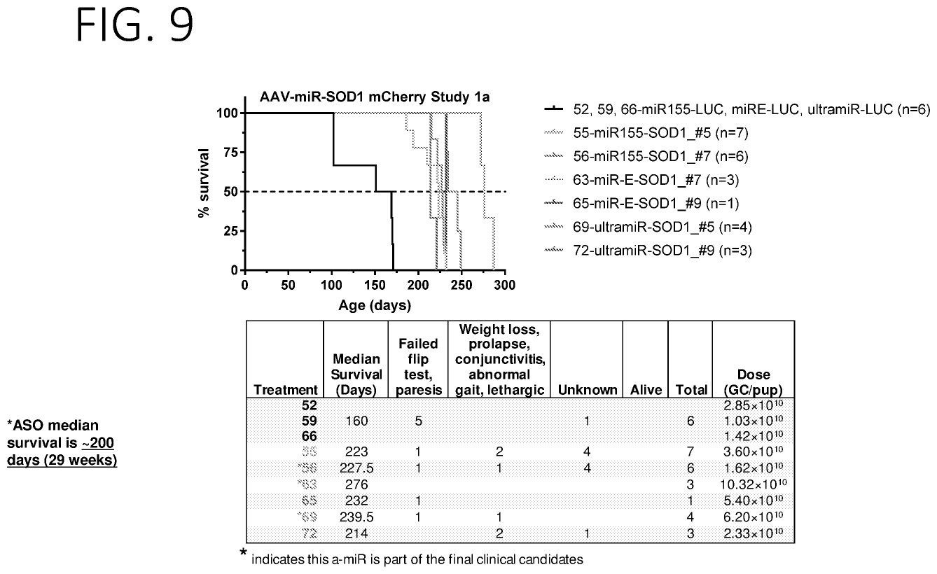

The C57BL/6J mice expressing the human SOD1-G93A transgene develop symptoms similar to ALS at roughly ≥7 weeks of age and succumb to the disease 14 to 29 weeks after birth. To determine whether reduction of SOD1 expression in these animals produces a benefit, animals were treated on P0 by ICV infusion of AAV9 encoding miR-155-SOD1-#2, miR-155-SOD1-#7, miR-E-SOD1-#7, or ultramiR-SOD1-#5 respectively. Compound muscle action potential (CMAP) was recorded in tibialis muscles roughly every 4 weeks from 5 weeks of age onward to assess the degree of muscle denervation and atrophy at electrophysiological level. In SOD1-G93A mice, CMAP declines over time. SOD1-G93A mice treated with all four a-miR candidates maintained CMAP over 32 weeks, indicating a sustained benefit after one-time administration of AAV9-a-miR (FIG. 8). Importantly, these treated mice did not show ALS-like phenotype at end stage (data not shown).

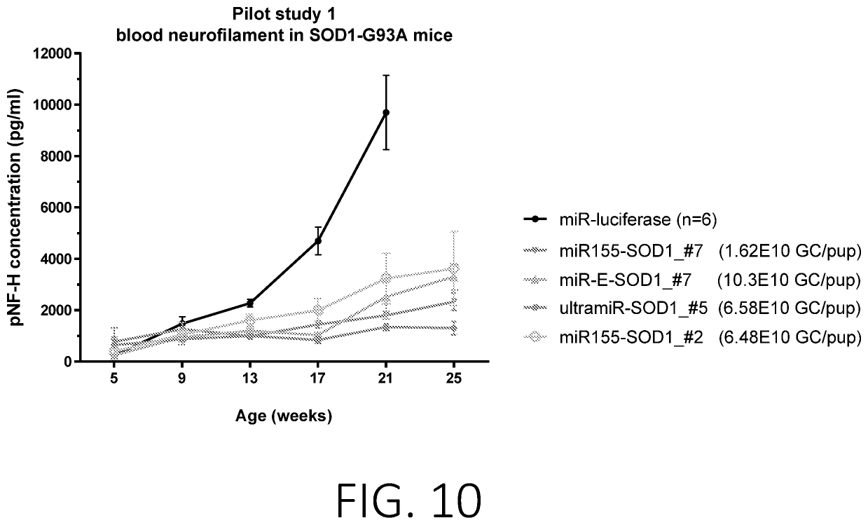

Serum phosphorylated neurofilament heavy chain (pNF-H) was quantified every 4 weeks by the ELLA microfluidic enzyme-linked immunosorbent assay (ELISA) platform to assess axonal damage or neuronal loss. Its level increases in serum in SOD1-G93A mice and human patients with ALS and has been used as a pharmacodynamic (PD) marker in clinic. Throughout 25 weeks in this study, SOD1-G93A mice treated with all four a-miR candidates showed lower levels of serum pNF-H compared with SOD1-G93A mice treated with control a-miR (FIG. 10), indicating robust protection against neuronal loss or axon degeneration. The data demonstrates mechanism of action (MOA) by a-miR-SOD1 in SOD1 inhibition and consequential alleviation of SOD1-G93A toxicity.

### Example 5: In Vivo Testing of AAV-miR-SOD1 Duplex

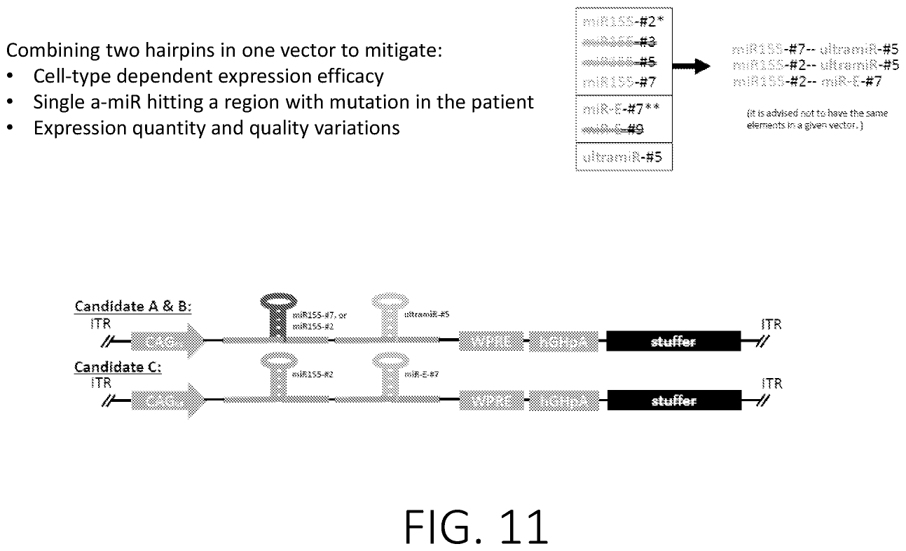

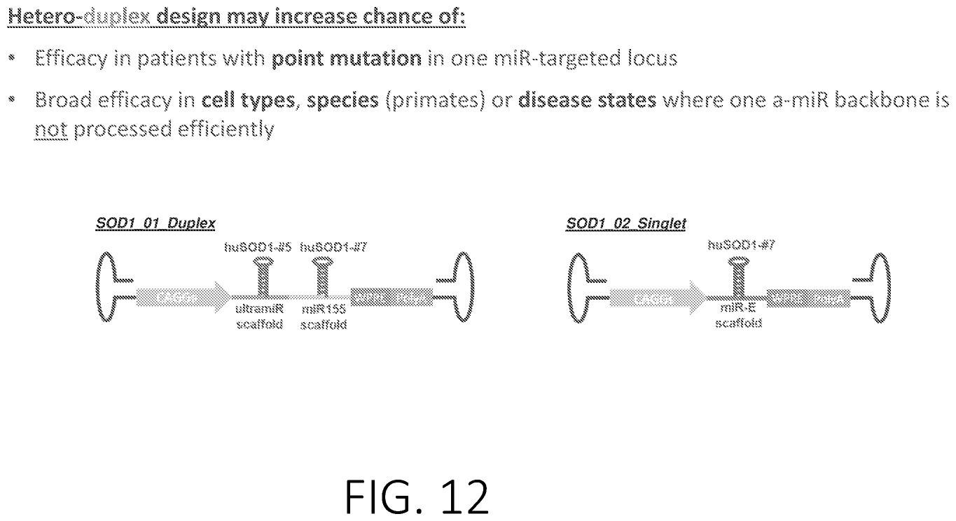

Two of the four a-miR-SOD1 candidates were further cloned into a single AAV9 vector to create hetero-duplex a-miR-SOD1 candidates, each of which consists of distinct guide strand sequences and distinct a-miR scaffolds (FIG. 11). The hetero-duplex a-miR-SOD1 design can ensure efficacy in patients with point mutation or SNP in the SOD1 gene locus targeted by 1 a-miR guide strand. The efficacy and safety of hetero-duplex a-miR candidates will be further assessed in additional nonclinical studies in mice and in non-human primates.

### Example 6: AAV Administration Methods for Reduced Toxicity

The present Example provides, among other things, methods of treating ALS that exhibit reduced toxicity and/or immunoreactivity comprising administration of inhibitory nucleic acids (e.g., in the form of rAAV) by intrathecal injection. In some embodiments, serum neurofilament (pNFH) measurement, and/or histopathological analysis of CNS tissues as well as peripheral organs, is used to assess the degree of toxicity of compositions and methods of the present disclosure and compositions and methods known in the art so they may be compared. The following exemplary methods are just one such example of administering inhibitory nucleic acids with reduced toxicity.

As described herein, the present disclosure identifies serum pNF-H as a particularly useful biomarker that can be used to quantify relative toxicity of rAAV compositions and administration methods. Accordingly, toxicity in subjects administered compositions of the present disclosure (e.g., rAAV compositions) can be compared to subjects receiving alternative compositions and/or compositions administered by a different route of administration. In particular, toxicity in subjects administered compositions of the present disclosure (e.g., rAAV compositions) by intrathecal injection can be compared to subjects receiving alternative compositions and/or compositions administered by a different route of administration. In some embodiments, administered compositions, e.g., compositions comprising inhibitory nucleic acids of the present disclosure, are compared to an appropriate control. In some embodiments, an appropriate control comprises a composition comparable to the administered composition being tested (e.g., empty vector, a known composition of known toxicity or known lack of toxicity, etc.). In some embodiments, serum pNF-H can be quantified in cells, tissues, or subjects at regular intervals (e.g., every 4 weeks) by any known method, e.g., by the ELLA microfluidic enzyme-linked immunosorbent assay (ELISA) platform, to assess axonal damage or neuronal loss. In some embodiments, a test composition or test method of administration can be determined to be more toxic than a known composition or known method of administration (e.g., a composition or method with known toxicity, or known lack of toxicity) when the level of pNF-H in a cell or tissue treated with a test composition or a test method is at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 25 fold, at least 30 fold, at least 35 fold, at least 40 fold, at least 45 fold, at least 50 fold, at least 55 fold, at least 60 fold, or at least 100 fold higher than a known composition or a known method. In some embodiments, a test composition or test method of administration can be determined to be more toxic than a known composition or known method of administration (e.g., a composition or method with known toxicity, or known lack of toxicity) when the level of pNF-H in a cell or tissue treated with a test composition or a test method is within a range of about 10 fold to 60 fold higher than a known composition or a known method.

By way of specific example, one such toxicity assessment may be made as follows. The present disclosure sets forth the surprising discovery that administration of rAAV by intrathecal injection exhibits reduced toxicity compared to other methods of administration. Intrathecal injection can be a performed in a subject by any method known in the art. In the present example, rodents are injected with rAAV compositions of the present disclosure once, or up to four times over two weeks. In this particular example, animals are anesthetized with isoflurane. Further, lack of response to toe/tail pinch is used to assess depth of anesthesia. All hair is clipped from the injection area, and ocular lubricant is applied. The animals are then placed on a heating source in ventral recumbency. The injection site is thoroughly cleaned, including three wipes with betadine and three wipes with isopropyl alcohol (alternating). To facilitate the intrathecal injection, the lumbar section of the animal may be raised on a bar to open up the intravertebral space. The needle is then inserted in the gap between L5 and L6. A 29G-30G X 1/2″ needle is used to administer the agent at slow rate as to minimize rapid changes in CSF pressure. When compositions of the present disclosure were delivered by intrathecal injections in wild-type mice, the same AAV-a-miR showed reduced blood pNFH of about a few hundred to below 2000 pg/ml compared to the range of 1000-6000 pg/ml of blood pNFH when delivered by P0 ICV.

### Example 7: In Vivo Testing of AAV-miR-SOD1 with Weaker Promoters

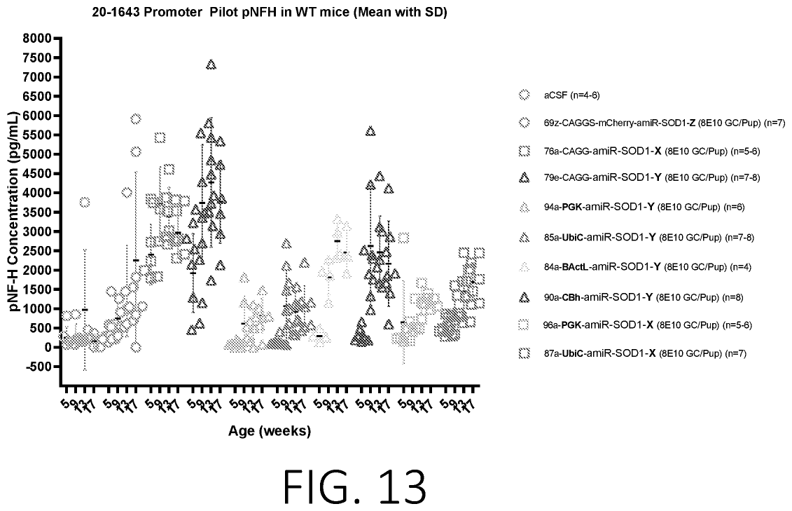

Previous studies performed in rodents, rabbits, and non-human primates have shown qualitative as well as semi-quantitative correlations between serum pNFH levels and the severity and incidence of histopathology findings in dorsal root ganglion (DRG) in response to AAV transduction. The present Example provides studies to determine whether the inclusion of weaker promoters (e.g., promoters that drive expression to an extent less than that observed for the CAG promoter) can ameliorate axonal damage associated with the above-mentioned DRG findings. Wild-type C57BL6/J mice were treated on P0 by ICV infusion of AAV9 encoding miR-SOD1 candidate X, Y or Z, whose expression is driven by either CAG, PGK, UbiC (Ubiquitin C), BActL (beta-actin long), or CBh promoter. Serum pNFH levels were quantified at 5, 9, 13 and 17 weeks after injection to assess axonal damage and/or neuronal loss. Animals treated with all vectors containing amiR-SOD1 X or Y driven by a weaker promoter (e.g., PGK, UbiC (Ubiquitin C), BActL (beta-actin long), or CBh) showed lower levels of serum pNFH compared with mice treated with the corresponding a-miR-SOD1 vectors driven instead by the CAG promoter (FIG. 13). The data indicate the capability of weaker promoters to ameliorate axonal damage associated with AAV overexpression in the DRG.

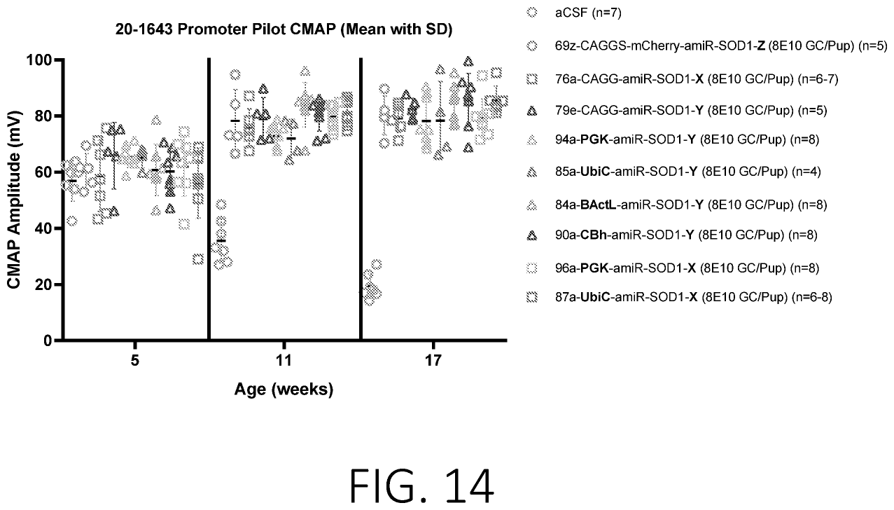

The C57BL6/J mice expressing the human SOD1-G93A transgene developed symptoms similar to ALS at roughly ≥7 weeks of age, and succumbed to the disease at 14 to 29 weeks of age. To determine whether reduction of SOD1 expression in these animals produces a benefit, mice were treated at P0 by ICV infusion of AAV9 encoding miR-SOD1 candidate X, Y or Z whose expression is driven by either CAG, PGK, UbiC (Ubiquitin C), BActL (beta-actin long) or CBh promoter. Compound muscle action potential (CMAP) was recorded in tibialis muscles at 5, 11 and 17 weeks of age to assess the degree of muscle denervation and atrophy at the electrophysiological level. In SOD1-G93A mice, CMAP declined over time. SOD1-G93A mice treated with any of the weaker promoter candidates maintained CMAP for over 17 weeks, indicating a sustained benefit after one-time administration of AAV9-a-miR (FIG. 14).

### Example 8: In Vivo Assessment of the Efficacy AAV-miR-SOD1 Vectors with Weaker Promoters

The present Example provides studies to assess safety and efficacy of AAV-miR-SOD1 vectors having weaker promoters (relative to the CAG promoter), as described in Example 7. A 23-week in-life study is conducted to determine the efficacy as well as safety profiles of AAV9-amiR-SOD1 vectors having weaker promoters (e.g., PGK, UbiC (Ubiquitin C), BActL (beta-actin long) or CBh promoter). C57BL6/J mice are administered AAV9-amiR-SOD1 vector via a single ICV injection at P0. Two dose levels are assessed, for example, doses of 1×10¹⁰ and 8×10¹⁰ GC/mouse. CMAP measurement, serum pNFH levels, SOD1 knockdown in spinal cord, and RNAseq analysis of DRG are evaluated.

A 4-week in-life study is conducted to determine dorsal root ganglion toxicity following a single intracisternal magna injection of AAV9-amiR-SOD1 vectors in the New Zealand white rabbit. Vectors are designed such that the transgene is driven by a weaker promoter as described above. In some embodiments, vectors may not comprise a WPRE. In some embodiments, vectors may comprise an altered polyA signal, such as a synthetic polyA sequence plus transcription pause site (e.g., a polyA signal having a nucleic acid sequence of SEQ ID NO: 64) from 5′ to 3′ on the minus strand. Post-necropsy, selected tissues are examined for histopathology, as well as for amiR-SOD1 expression via biochemical, genomic, and/or histological methods.

### REFERENCES

Akimoto C, Morita M, Atsuta N, et al. High-Resolution Melting (HRM) Analysis of the Cu/Zn Superoxide Dismutase (SOD1) Gene in Japanese Sporadic Amyotrophic Lateral Sclerosis (SALS) Patients. Neurol Res Int. 2011; 2011:165415. Epub 2011/04/12.
Arnesano F, Banci L, Bestini I, Martinelli M, Furukawa Y, et al. (2004) The unusually stable quaternary structure of human SOD1 is controlled by both metal occupancy and disulfide status. J Biol Chem 279: 47998-48003.
Auyeung V C, Ulitsky I, McGeary S E, Bartel D P. Beyond secondary structure: primary-sequence determinants license pri-miRNA hairpins for processing. Cell. 2013; 152(4):844-858. doi:10.1016/j.cell.2013.01.031Bertini I, Mangani S, Viezzoli M S (1998) Advanced Inorganic Chemistry. San Diego: Academic Press. pp 127-250.
Byrne S, Walsh C, Lynch C, et al. Rate of familial amyotrophic lateral sclerosis: a systematic review and meta-analysis. J Neurol Neurosurg Psychiatry. 2011; 82(6):623-7. Epub 2010/11/03.
Calcedo R, Morizono H, Wang L, McCarter R, He J, et al. 2011. Adeno-associated virus antibody profiles in newborns, children, and adolescents. Clin. Vaccine Immunol. 18:1586-88.
Calcedo R, Vandenberghe L H, GaoG, Lin J, Wilson J M. 2009. Worldwide epidemiology of neutralizing antibodies to adeno-associated viruses. J. Infect. Dis. 199:381-90.
Chiò A, Traynor B J, Lombardo F, et al. Prevalence of SOD1 mutations in the Italian ALS population. Neurology. 2008; 70(7):533-7.
Chiò A, Logroscino G, Traynor B, et al. Global epidemiology of amyotrophic lateral sclerosis (ALS): a systematic review of the literature. Presented at the 23rd International Symposium on ALS/MND; Dec. 5-7, 2012; Chicago, IL.
Chiò A, Calvo A, Mazzini L, et al. Extensive genetics of ALS: a population-based study in Italy. Neurology. 2012; 79(19):1983-9. Epub 2012/10/24.
Clément N, Grieger J C. Manufacturing of recombinant adeno-associated viral vectors for clinical trials. Mol Ther Methods Clin Dev. 2016; 3:16002
Culotta V C, Yang M, O'Halloran T V (2006) Activation of superoxide dismutases: putting the metal to the pedal. Biochim Biophys Acta 1763: 747-758.
Dangoumau A, Verschueren A, Hammouche E, Papon M A, Blasco H, Cherpi-Antar C, et al. Novel SOD1 mutation p.V31A identified with a slowly progressive form of amyotrophic lateral sclerosis. Neurobiol Aging 2014; 35:266.e1-4.
De Vos K, Severin F, Van Herreweghe F, Vancompernolle K, Goossens V, Hyman A, et al. Tumor necrosis factor induces hyperphosphorylation of kinesin light chain and inhibits kinesin-mediated transport of mitochondria. J Cell Biol 2000; 149:1207-14.
De Vos K J, Chapman A L, Tennant M E, Manser C, Tudor E L, Lau K F, et al. Familial amyotrophic lateral sclerosis-linked SOD1 mutants perturb fast axonal transport to reduce axonal mitochondria content. Hum Mol Genet 2007; 16:2720-8.
Donnelly C J, Zhang P W, Pham J T, Haeusler A R, Mistry N A, Vidensky S, et al. RNA toxicity from the ALS/FTD C9ORF72 expansion is mitigated by antisense intervention. Neuron 2013; 80:415-28.
Fellmann C, Hoffmann T, Sridhar V, et al. An optimized microRNA backbone for effective single-copy RNAi. Cell Rep. 2013; 5(6):1704-1713.
Forsberg K, Andersen P M, Marklund S L, Brännström T. Glial nuclear aggregates of superoxide dismutase-1 are regularly present in patients with amyotrophic lateral sclerosis. Acta Neuropathol 2011; 121:623-34.
Fowler D K, Williams C, Gerritsen A T, Washbourne P. Improved knockdown from artificial microRNAs in an enhanced miR-155 backbone: a designer's guide to potent multi-target RNAi. Nucleic Acids Res. 2016; 44(5):e48. doi:10.1093/nar/gkv1246
Fridovich I (1978) The biology of oxygen radicals. Science 201: 875-879.
Grieger J C, Soltys S M, Samulski R J. Production of Recombinant Adeno-associated Virus Vectors Using Suspension HEK293 Cells and Continuous Harvest of Vector From the Culture Media for GMP FIX and FLT1 Clinical Vector. Mol Ther. 2016; 24(2):287-297.
Hauswirth W W, Berns K I. 1977. Origin and termination of adeno-associated virus DNA replication. Virology 78:488-99
Ivanova M I, Sievers S A, Guenther E L, Johnson L M, Winkler D D, Galaleldeen A, et al. Aggregation-triggering segments of SOD1 fibril formation support a common pathway for familial and sporadic ALS. Proc Natl Acad Sci USA 2014; 111:197-201.
Jaiswal M K. Selective vulnerability of motoneuron and perturbed mitochondrial calcium homeostasis in amyotrophic lateral sclerosis: Implications for motoneurons specific calcium dysregulation. Mol Cell Ther 2014; 2:26.
Magrané J, Manfredi G. Mitochondrial function, morphology, and axonal transport in amyotrophic lateral sclerosis. Antioxid Redox Signal 2009; 11:1615-26.
Martin J, Frederick A, Luo Y, et al. Generation and characterization of adeno-associated virus producer cell lines for research and preclinical vector production. Hum Gene Ther Methods. 2013; 24(4):253-69.
Mehta P, Antao V, Kaye W, et al. Prevalence of amyotrophic lateral sclerosis—United States, 2010-2011. MMWR Suppl. 2014; 63(7):1-14.
McLaughlin S K, Collis P, Hermonat P L, Muzyczka N. 1988. Adeno-associated virus general transduction vectors: analysis of proviral structures. J. Virol. 62:1963-73
Mitsumoto H, Factor-Litvak P, Andrews H, Goetz R R, Andrews L, Rabkin J G, et al. ALS Multicenter Cohort Study of Oxidative Stress (ALS COSMOS): Study methodology, recruitment, and baseline demographic and disease characteristics. Amyotroph Lateral Scler Frontotemporal Degener 2014; 15:192-203.
Pasinelli P, Belford M E, Lennon N, Bacskai B J, Hyman B T, Trotti D, et al. Amyotrophic lateral sclerosis-associated SOD1 mutant proteins bind and aggregate with Bcl-2 in spinal cord mitochondria. Neuron 2004; 43:19-30.
Pelossof, R., Fairchild, L., Huang, C. et al. Prediction of potent shRNAs with a sequential classification algorithm. Nat Biotechnol 35, 350-353 (2017).
Renton A E, Chiò A, Traynor B J. State of play in amyotrophic lateral sclerosis genetics. Nat Neurosci. 2014; 17(1):17-23. Epub 2013/12/26.
Samulski R J, Chang L S, Shenk T. 1989. Helper-free stocks of recombinant adeno-associated viruses: Normal integration does not require viral gene expression. J. Virol. 63:3822-28
Shi P, Ström A L, Gal J, Zhu H. Effects of ALS-related SOD1 mutants on dynein- and KIF5-mediated retrograde and anterograde axonal transport. Biochim Biophys Acta 2010; 1802:707-16.
Valentine J S, Doucette P A, Potter S Z (2005) Copper-Zinc Superoxide Dismutase and Amyotrophic Lateral Sclerosis. Annu Rev Biochem 74: 563-593.
Vande Velde C, Miller T M, Cashman N R, Cleveland D W. Selective association of misfolded ALS-linked mutant SOD1 with the cytoplasmic face of mitochondria. Proc Natl Acad Sci USA 2008; 105:4022-7.
Vucic S, Kiernan M C. Abnormalities in cortical and peripheral excitability in flail arm variant amyotrophic lateral sclerosis. J Neurol Neurosurg Psychiatry 2007; 78:849-52.
Wang W, Zhang F, Li L, Tang F, Siedlak S L, Fujioka H, et al. MFN2 couples glutamate excitotoxicity and mitochondrial dysfunction in motor neurons. J Biol Chem 2015; 290:168-82.
Zhu Y B, Sheng Z H. Increased axonal mitochondrial mobility does not slow amyotrophic lateral sclerosis (ALS)-like disease in mutant SOD1 mice. J Biol Chem 2011; 286:23432-40.

What is claimed is:

1. A recombinant adeno-associated virus (rAAV) vector comprising:
a) a modified AAV genome comprising:
(i) a promoter; and
(ii) at least two or more different miRNA sequences; and
b) a capsid;
wherein each of the two or more miRNA sequences comprise a guide strand sequence that targets superoxide dismutase 1 (SOD1), and a scaffold sequence and wherein each of the two or more miRNA sequences are operably linked to the promoter.

2. The rAAV vector of claim 1, wherein at least two miRNA sequences comprise at least one guide strand sequence that shares at least 80% sequence identity to SEQ ID NO: 2 and at least one guide strand sequence that shares at least 80% sequence identity to SEQ ID NO: 5.

3. The rAAV vector of claim 1 or claim 2, wherein at least two miRNA sequences comprise at least one guide strand sequence that includes at least 5 (e.g., at least 10, at least 15, at least 20, etc.) contiguous nucleotides with reference to SEQ ID NO: 2 and at least one guide strand sequence that includes at least 5 (e.g., at least 10, at least 15, at least 20, etc.) contiguous nucleotides with reference to SEQ ID NO: 5.

4. The rAAV vector of any one of claims 1-3, wherein at least two miRNA sequences comprise at least one guide strand sequence comprising SEQ ID NO: 2 and at least one guide strand sequence comprising SEQ ID NO: 5.

5. The rAAV vector of any one of claims 1-4, wherein at least one miRNA sequence comprises a scaffold sequence that shares at least 80% sequence identity to SEQ ID NO: 16.

6. The rAAV vector of any one of claims 1-5, wherein at least one miRNA sequence comprises a scaffold sequence comprising SEQ ID NO: 16.

7. The rAAV vector of any one of claims 1-6, wherein at least one miRNA sequence comprises a scaffold sequence that shares at least 80% sequence identity to SEQ ID NO: 18.

8. The rAAV vector of any one of claims 1-7, wherein at least one miRNA sequence comprises a scaffold sequence comprising SEQ ID NO: 18.

9. The rAAV vector of any one of claims 1-8, wherein at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 5.

10. The rAAV vector of any one of claims 1-9, wherein at least two miRNA sequences comprise one miRNA sequence with a guide strand sequence comprising SEQ ID NO: 2, and one miRNA sequence with a guide strand sequence comprising SEQ ID NO: 5 and a scaffold sequence comprising SEQ ID NO: 18.

11. The rAAV vector of any one of claims 1-10, wherein at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 5 and a scaffold sequence comprising SEQ ID NO: 18.

12. The rAAV vector of claim 1, wherein at least two miRNA sequences comprise at least one guide strand sequence that shares at least 80% sequence identity to SEQ ID NO: 2 and at least one guide strand sequence that shares at least 80% sequences identity to SEQ ID NO: 7.

13. The rAAV vector of claim 1 or claim 12, wherein at least two miRNA sequences comprise at least one guide strand sequence that includes at least 5 (e.g., at least 10, at least 15, at least 20, etc.) contiguous nucleotides with reference to SEQ ID NO: 2 and at least one guide strand sequence that includes at least 5 (e.g., at least 10, at least 15, at least 20, etc.) contiguous nucleotides with reference to SEQ ID NO: 7.

14. The rAAV vector of any one of claims 1, 12, or 13, wherein at least two miRNA sequences comprise at least one guide strand sequence comprising SEQ ID NO: 2 and at least one guide strand sequence comprising SEQ ID NO: 7.

15. The rAAV vector of any one of claims 1 or 12-14, wherein at least one miRNA sequence comprises a scaffold sequence that shares at least 80% sequence identity to SEQ ID NO: 16.

16. The rAAV vector of any one of claims 1 or 12-15, wherein at least one miRNA sequence comprises a scaffold sequence comprising SEQ ID NO: 16.

17. The rAAV vector of any one of claims 1 or 12-16, wherein at least one miRNA sequence comprises a scaffold sequence that shares at least 80% sequence identity to SEQ ID NO: 17.

18. The rAAV vector of any one of claims 1 or 12-17, wherein at least one miRNA sequence comprises a scaffold sequence comprising SEQ ID NO: 17.

19. The rAAV vector of any one of claims 1 or 12-18, wherein at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 7.

20. The rAAV vector of any one of claims 1 or 12-19, wherein at least two miRNA sequences comprise one miRNA sequence with a guide strand sequence comprising SEQ ID NO: 2, and one miRNA sequence with a guide strand sequence comprising SEQ ID NO: 7 and a scaffold sequence comprising SEQ ID NO: 17.

21. The rAAV vector of any one of claims 1 or 12-18, wherein at least two miRNA sequences comprise one miRNA sequence with a guide strand sequence comprising SEQ ID NO: 2 and a scaffold sequence comprising SEQ ID NO: 16, and one miRNA sequence with a guide strand sequence comprising SEQ ID NO: 7 and a scaffold sequence comprising SEQ ID NO: 17.

22. The rAAV vector of claim 1, wherein the two miRNA sequences comprise at least one guide strand sequence that shares at least 80% sequence identity to SEQ ID NO: 5 and at least one guide strand sequence that shares at least 80% identity to SEQ ID NO: 7.

23. The rAAV vector of claim 1 or 22, wherein at least two miRNA sequences comprise at least one guide strand sequence that includes at least 5 (e.g., at least 10, at least 15, at least 20, etc.) contiguous nucleotides with reference to SEQ ID NO: 5 and at least one guide strand sequence that includes at least 5 (e.g., at least 10, at least 15, at least 20, etc.) contiguous nucleotides with reference to SEQ ID NO: 7.

24. The rAAV vector of any one of claims 1, 22, or 23, wherein at least two miRNA sequences comprise at least one guide strand sequence comprising SEQ ID NO: 5 and at least one guide strand sequence comprising SEQ ID NO: 7.

25. The rAAV vector of any one of claims 1 or 22-24, wherein at least one miRNA sequence comprises a scaffold sequence that shares at least 80% sequence identity to SEQ ID NO: 18.

26. The rAAV vector of any one of claims 1 or 22-25, wherein at least one miRNA sequence comprises a scaffold sequence comprising SEQ ID NO: 18.

27. The rAAV vector of any one of claims 1 or 22-26, wherein at least one miRNA sequence comprises a scaffold sequence that shares at least 80% sequence identity to SEQ ID NO: 16.

28. The rAAV vector of any one of claims 1 or 22-27, wherein at least one miRNA sequence comprises a scaffold sequence comprising SEQ ID NO: 16.

29. The rAAV vector of any one of claims 1 or 22-28, wherein at least two miRNA sequences comprise one miRNA sequence with a guide strand sequence comprising SEQ ID NO: 5 and a scaffold sequence comprising SEQ ID NO: 18, and one miRNA sequence with a guide strand sequence comprising SEQ ID NO: 7.

30. The rAAV vector of any one of claims 1 or 22-29, wherein at least two miRNA sequences comprise one miRNA sequence with a guide strand sequence comprising SEQ ID NO: 5, and one miRNA sequence with a guide strand sequence comprising SEQ ID NO: 7 and a scaffold sequence comprising SEQ ID NO: 16.

31. The rAAV vector of any one of claims 1 or 22-30, wherein at least two miRNA sequences comprise one miRNA sequence with a guide strand sequence comprising SEQ ID NO: 5 and a scaffold sequence comprising SEQ ID NO: 18, and one miRNA sequence with a guide strand sequence comprising SEQ ID NO: 7 and a scaffold sequence comprising SEQ ID NO: 16.

32. The rAAV vector of claim 1, wherein the modified AAV genome comprises at least three miRNA guide sequences.

33. The rAAV vector of claim 32, wherein at least three miRNA guide sequences comprise at least one guide strand sequence that shares at least 80% sequence identity to SEQ ID NO: 2, at least one guide strand sequence that shares at least 80% identity to SEQ ID NO: 5, and at least one guide strand sequence that shares at least 80% identity to SEQ ID NO: 7.

34. The rAAV vector of claim 32 or 33, wherein at least three miRNA guide sequences comprise at least one guide strand sequence that includes at least 5 (e.g., at least 10, at least 15, at least 20, etc.) contiguous nucleotides with reference to SEQ ID NO: 2, at least one guide strand sequence that includes at least 5 (e.g., at least 10, at least 15, at least 20, etc.) contiguous nucleotides with reference to SEQ ID NO: 5, and at least one guide strand sequence that includes at least 5 (e.g., at least 10, at least 15, at least 20, etc.) contiguous nucleotides with reference to SEQ ID NO: 7.

35. The rAAV vector of any one of claims 32-34 wherein the at least three miRNA guide sequences comprise at least one guide strand sequence comprising SEQ ID NO: 2, at least one guide strand sequence comprising SEQ ID NO: 5, and at least one guide strand sequence comprising SEQ ID NO: 7.

36. The rAAV vector of any one of claims 32-35, wherein at least one miRNA sequence comprises a scaffold sequence that shares at least 80% sequence identity to SEQ ID NO: 16.

37. The rAAV vector of any one of claims 32-36, wherein at least one miRNA sequence comprises a scaffold sequence comprising SEQ ID NO: 16.

38. The rAAV vector of any one of claims 32-37, wherein at least one miRNA sequence comprises a scaffold sequence that shares at least 80% sequence identity to SEQ ID NO: 18.

39. The rAAV vector of any one of claims 32-38, wherein at least one miRNA sequence comprises a scaffold sequence comprising SEQ ID NO: 18.

40. The rAAV vector of any one of claims 32-39, wherein at least three miRNA sequence comprise at least one miRNA sequence with a guide strand sequence comprising SEQ ID NO: 2 and a scaffold sequence that shares at least 80% sequence identity to SEQ ID NO: 16, and at least one miRNA sequence with a guide strand sequence comprising SEQ ID NO: 5 and a scaffold sequence that shares at least 80% sequence identity to SEQ ID NO: 18.

41. The rAAV vector of any one of claims 32-40, wherein at least three miRNA sequences comprise at least one miRNA sequence with a guide strand sequence comprising SEQ ID NO: 2 and a scaffold sequence comprising SEQ ID NO: 16, and at least one miRNA sequence with a guide strand sequence comprising SEQ ID NO: 5 and a scaffold sequence comprising SEQ ID NO: 18.

42. The rAAV vector of any one of claims 32-41, wherein at least three miRNA sequences comprise at least one miRNA sequence with a guide strand sequence comprising SEQ ID NO: 2 and a scaffold sequence that shares at least 80% sequence identity to SEQ ID NO: 16, and at least one miRNA sequence with a guide strand sequence comprising 7 and a scaffold sequence that shares at least 80% sequence identity to SEQ ID NO: 17.

43. The rAAV vector of any one of claims 32-42, wherein at least three miRNA sequences comprise at least one miRNA sequence with a guide strand sequence comprising SEQ ID NO: 2 and a scaffold sequence comprising SEQ ID NO: 16, and at least one miRNA sequence with a guide strand sequence comprising SEQ ID NO: 7 and a scaffold sequence comprising SEQ ID NO: 17.

44. The rAAV vector of any one of claims 32-43, wherein at least three miRNA sequences comprise at least one miRNA sequence with a guide strand sequence comprising SEQ ID NO: 5 and a scaffold sequence that shares at least 80% sequence identity to SEQ ID NO: 18, and at least one miRNA sequence with a guide strand sequence comprising SEQ ID NO: 7 and a scaffold sequence that shares at least 80% sequence identity to SEQ ID NO: 16.

45. The rAAV vector of any one of claims 32-44, wherein at least three miRNA sequences comprise at least one miRNA sequence with a guide strand sequence comprising SEQ ID NO: 5 and a scaffold sequence comprising SEQ ID NO: 18, and at least one miRNA sequence with a guide strand sequence comprising SEQ ID NO: 7 and a scaffold sequence comprising SEQ ID NO: 16.

46. The rAAV vector of any one of claims 32-45, wherein at least three miRNA sequences comprise at least one miRNA sequence with a guide strand sequence comprising SEQ ID NO: 5 and a scaffold sequence that shares at least 80% sequence identity to SEQ ID NO: 18, and at least one miRNA sequence with a guide strand sequence comprising SEQ ID NO: 7 and a scaffold sequence that shares at least 80% sequence identity to SEQ ID NO: 17.

47. The rAAV vector of any one of claims 32-46, wherein at least three miRNA sequences comprise at least one miRNA sequence with a guide strand sequence comprising SEQ ID NO: 5 and a scaffold sequence comprising SEQ ID NO: 18, and at least one miRNA sequence with a guide strand sequence comprising SEQ ID NO: 7 and a scaffold sequence comprising SEQ ID NO: 17.

48. The rAAV vector of claim 32-47, wherein at least three miRNA sequences comprise one miRNA sequence with a guide strand sequence comprising SEQ ID NO: 2 and a scaffold sequence comprising SEQ ID NO: 16, one miRNA sequence with a guide strand sequence comprising SEQ ID NO: 5 and a scaffold sequence comprising SEQ ID NO: 18, and one miRNA sequence with a guide strand sequence comprising SEQ ID NO: 7 and a scaffold sequence comprising SEQ ID NO: 17.

49. A recombinant adeno-associated virus (rAAV) vector comprising:
a) a modified AAV genome comprising:
(i) a promoter;
(ii) at least one miRNA sequence; and
b) a capsid;
wherein at least one miRNA sequence comprises a guide strand sequence comprising SEQ ID NO: 2 and a miR-155 scaffold sequence, and wherein the miRNA sequence is operably linked to the promoter.

50. A recombinant adeno-associated virus (rAAV) vector comprising:
a) a modified AAV genome comprising:
(i) a promoter;
(ii) at least one miRNA sequence; and
b) a capsid;
wherein at least one miRNA sequence comprises a guide strand sequence comprising SEQ ID NO: 5 and a scaffold sequence, and wherein the miRNA sequence is operably linked to the promoter.

51. The rAAV vector of claim 50, wherein the scaffold sequence comprises SEQ ID NO: 18.

52. A recombinant adeno-associated virus (rAAV) vector comprising:
a) a modified AAV genome comprising:
(i) a promoter;
(ii) at least one miRNA sequence; and
b) a capsid;
wherein at least one miRNA sequence comprises a guide strand sequence comprising SEQ ID NO: 7 and a scaffold sequence and wherein the miRNA sequence is operably linked to the promoter.

53. The rAAV vector of claim 52, wherein the scaffold sequence comprises SEQ ID NO: 16, or SEQ ID NO: 17.

54. The rAAV vector of any one of claims 1-53, wherein the capsid is of serotype AAV1, AAV2, AAV3, AAV4, AAV5, AAV6, AAV7, AAV8, AAV9, AAV10, AAV11, AAV12, or variants or combinations thereof.

55. The rAAV vector of claim 54, wherein the capsid is or comprises AAV9.

56. The rAAV vector of claim 55, wherein the capsid is or comprises AAVrh.10.

57. The rAAV vector of any one of claims 1-56, wherein the modified AAV genome further comprises a nucleic acid sequence encoding a reporter protein.

58. The rAAV vector of claim 57, wherein the reporter protein is a luciferase protein, RFP, mCherry protein, GFP, or any variant and/or combination thereof.

59. The rAAV vector of claim 57 or 58, wherein the reporter protein is mCherry.

60. The rAAV vector of claim 57 or 58, wherein the reporter protein is GFP or a GFP variant.

61. The rAAV vector of any one of claims 1-60, wherein the promoter is CMV, EF1a, SV40, PGK, PGK1, Ubc, human beta-actin, beta-actin long (BActL), CAG, CBA, CBh, TRE, U6, H1, 7SK, ubiquitin C (UbiC), and any variant and/or combination thereof.

62. The rAAV vector of claim 61, wherein the promoters is CAG, CMV, Synapsin, GFAP, or any combination thereof.

63. The rAAV vector of claim 61, wherein the promoter is a Pol II promoter.

64. The rAAV vector of claim 61, wherein the promoter is a Pol III promoter.

65. The rAAV vector of any one of claims 1-64, wherein the modified AAV genome further comprises a 3′ UTR element that enhances expression.

66. The rAAV vector of claim 65, wherein the 3′UTR element is a miRNA response element (MRE), AU-rich element (ARE), poly-A tail, WPRE, bGH, hGH, or any combination thereof.

67. The rAAV vector of claim 65, wherein the 3′UTR element is WPRE, bGH, hGH, p(A), or any combination thereof.

68. The rAAV vector of any one of claims 1-67, wherein the rAAV vector provides a guide strand to passenger strand ratio that is greater than 2.

69. The rAAV vector of any one of claims 1-68, wherein the rAAV vector provides a guide strand production level that is at least 0.01%, at least 0.1%, at least 1%, at least 2%, at least 3%, at least 4%, at least 5%, at least 10%, at least 15%, at least 20%, at least 25%, at least 30%, or at least 35%.

70. The rAAV vector of any one of claims 1-69, wherein the rAAV vector provides a guide strand potency that is greater than 50%.

71. The rAAV vector of any one of claims 1-70, wherein the rAAV vector provides a guide strand accuracy that is greater than 80%.

72. A pharmaceutical composition comprising an rAAV vector described in any one of the previous claims.

73. A nucleic acid encoding a modified AAV genome described in any one of the previous claims.

74. A vector comprising the nucleic acid of claim 73.

75. A method of treating a subject with Amyotrophic Lateral Sclerosis (ALS), the method comprising a step of:
administering a therapeutically effective amount of a composition that provides a recombinant adeno-associated virus (rAAV) vector that reduces SOD1 expression, wherein the rAAV vector is as described in any of one of the above claims.

76. A method of treating a subject with Amyotrophic Lateral Sclerosis (ALS), the method comprising a step of:
administering a therapeutically effective amount of a composition that provides a recombinant adeno-associated virus (rAAV) vector, wherein the rAAV vector comprises:
(a) a modified AAV genome comprising:
(i) a promoter; and
(ii) two or more different miRNA sequences; and
(b) a capsid;
wherein each of the two or more miRNA sequences comprise a guide strand that targets SOD1, and a scaffold sequence, and wherein each of the two or more miRNA sequences are operably linked to the promoter.

77. A method for simultaneously delivering two or more anti-SOD1 miRNAs to CNS tissue in a subject, the method comprising a step of:
administering a therapeutically effective amount of a composition that provides a recombinant adeno-associated virus (rAAV) vector, wherein the rAAV vector comprises:
(a) a modified AAV genome comprising:
(i) a promoter; and
(ii) two or more different miRNA sequences; and
(b) a capsid;
wherein each of the two or more miRNA sequences comprise a guide strand that targets SOD1, and a scaffold sequence, and wherein each of the two or more miRNA sequences are operably linked to the promoter.

78. The method of any one of claims 75-77, wherein the therapeutically effective amount comprises an amount between a minimally effective amount and a maximally tolerable amount of the pharmaceutical composition.

79. The method of any one of claims 75-78, wherein the minimally effective amount comprises an amount of the pharmaceutical composition sufficient to reduce the level of SOD1 in a target tissue.

80. The method of any one of claims 75-79, wherein the composition is administered by intravenous administration, intrathecal administration, intracisternal administration, intramuscular administration, or combinations thereof.

81. The method of any one of claims 75-80, wherein the capsid is of serotype AAV1, AAV2, AAV3, AAV4, AAV5, AAV6, AAV7, AAV8, AAV9, AAV10, AAV11, AAV12, or variants or combinations thereof.

82. A method of inhibiting SOD1 expression in a cell, the method comprising a step of:
administering a composition that provides a recombinant adeno-associated virus (rAAV) vector, wherein the rAAV vector comprises:
(a) a modified AAV genome comprising:
(i) a promoter; and
(ii) two or more different miRNA sequences; and
(b) a capsid;
wherein each of the two or more miRNA sequences comprise a guide strand that targets SOD1, and a scaffold sequence, and wherein each of the two or more miRNA sequences are operably linked to the promoter.

83. A recombinant adeno-associated virus (rAAV) vector comprising:
a) a modified AAV genome comprising:
(i) a promoter; and
(ii) one or more miRNA sequences; and
b) a capsid;
wherein the one or more miRNA sequences comprise a guide strand sequence that targets SOD1, and a scaffold sequence and wherein the one or more miRNA sequences are operably linked to the promoter.

84. The rAAV vector of claim 83, wherein one or more miRNA sequences comprise one or more guide strand sequences that share at least 80% sequence identity to a sequence selected from SEQ ID NOs: 1-12.

85. A recombinant adeno-associated virus (rAAV) vector comprising a modified AAV genome comprising:
(i) a promoter; and
(ii) at least two or more different miRNA sequences,
wherein each of the two or more miRNA sequences comprise a guide strand sequence that targets superoxide dismutase 1 (SOD1), and a scaffold sequence and wherein each of the two or more miRNA sequences are operably linked to the promoter.

86. A recombinant adeno-associated virus (rAAV) vector comprising a modified AAV genome comprising:
(i) a promoter; and
(ii) at least one miRNA sequence,
wherein at least one miRNA sequence comprises a guide strand sequence comprising SEQ ID NO: 2 and a miR-155 scaffold sequence, and wherein the miRNA sequence is operably linked to the promoter.

87. A recombinant adeno-associated virus (rAAV) vector comprising a modified AAV genome comprising:
(i) a promoter; and
(ii) at least one miRNA sequence,
wherein at least one miRNA sequence comprises a guide strand sequence comprising SEQ ID NO: 5 and a scaffold sequence, and wherein the miRNA sequence is operably linked to the promoter.

88. A recombinant adeno-associated virus (rAAV) vector comprising a modified AAV genome comprising:
(i) a promoter; and
(ii) at least one miRNA sequence,
wherein at least one miRNA sequence comprises a guide strand sequence comprising SEQ ID NO: 7 and a scaffold sequence and wherein the miRNA sequence is operably linked to the promoter.

89. A method of treating a subject with Amyotrophic Lateral Sclerosis (ALS), the method comprising a step of:
administering a therapeutically effective amount of a composition that provides a recombinant adeno-associated virus (rAAV) vector, wherein the rAAV vector comprises a modified AAV genome comprising:
(i) a promoter; and
(ii) two or more different miRNA sequences,
wherein each of the two or more miRNA sequences comprise a guide strand that targets SOD1, and a scaffold sequence, and wherein each of the two or more miRNA sequences are operably linked to the promoter.

90. A method for simultaneously delivering two or more anti-SOD1 miRNAs to CNS tissue in a subject, the method comprising a step of:
administering a therapeutically effective amount of a composition that provides a recombinant adeno-associated virus (rAAV) vector, wherein the rAAV vector comprises a modified AAV genome comprising:
(i) a promoter; and
(ii) two or more different miRNA sequences,
wherein each of the two or more miRNA sequences comprise a guide strand that targets SOD1, and a scaffold sequence, and wherein each of the two or more miRNA sequences are operably linked to the promoter.

91. A method of inhibiting SOD1 expression in a cell, the method comprising a step of:
administering a composition that provides a recombinant adeno-associated virus (rAAV) vector, wherein the rAAV vector comprises a modified AAV genome comprising:
(i) a promoter; and
(ii) two or more different miRNA sequences,
wherein each of the two or more miRNA sequences comprise a guide strand that targets SOD1, and a scaffold sequence, and wherein each of the two or more miRNA sequences are operably linked to the promoter.

92. A recombinant adeno-associated virus (rAAV) vector comprising a modified AAV genome comprising:
(i) a promoter; and
(ii) one or more miRNA sequences,
wherein the one or more miRNA sequences comprise a guide strand sequence that targets SOD1, and a scaffold sequence and wherein the one or more miRNA sequences are operably linked to the promoter.


SEQUENCE LISTING (71 sequences)

SEQ ID NO: 1 - DNA, 21 bp, Artificial Sequence
  Description of Artificial Sequence Synthetic oligonucleotide
tctgctcgaaattgatgatgc

SEQ ID NO: 2 - DNA, 21 bp, Artificial Sequence
  Description of Artificial Sequence Synthetic oligonucleotide
attactttccttctgctcgaa

SEQ ID NO: 3 - DNA, 21 bp, Artificial Sequence
  Description of Artificial Sequence Synthetic oligonucleotide
atgaacatggaatccatgcag

SEQ ID NO: 4 - DNA, 21 bp, Artificial Sequence
  Description of Artificial Sequence Synthetic oligonucleotide
ttcaatagacacatcggccac

SEQ ID NO: 5 - DNA, 21 bp, Artificial Sequence
  Description of Artificial Sequence Synthetic oligonucleotide
tactttcttcatttccacctt

SEQ ID NO: 6 - DNA, 21 bp, Artificial Sequence
  Description of Artificial Sequence Synthetic oligonucleotide
tttgtactttcttcatttcca

SEQ ID NO: 7 - DNA, 21 bp, Artificial Sequence
  Description of Artificial Sequence Synthetic oligonucleotide
tcaggatacatttctacagct

SEQ ID NO: 8 - DNA, 21 bp, Artificial Sequence
  Description of Artificial Sequence Synthetic oligonucleotide
ttatcaggatacatttctaca

SEQ ID NO: 9 - DNA, 21 bp, Artificial Sequence
  Description of Artificial Sequence Synthetic oligonucleotide
ttacagtgtttaatgtttatc

SEQ ID NO: 10 - DNA, 21 bp, Artificial Sequence
  Description of Artificial Sequence Synthetic oligonucleotide
tacacttttaagattacagtg

SEQ ID NO: 11 - DNA, 21 bp, Artificial Sequence
  Description of Artificial Sequence Synthetic oligonucleotide
aatgacaaagaaattctgaca

SEQ ID NO: 12 - DNA, 21 bp, Artificial Sequence
  Description of Artificial Sequence Synthetic oligonucleotide
tttagtttgaatttggattct

SEQ ID NO: 13 - DNA, 21 bp, Unknown
  Description of Unknown LUC control target sequence
ccggctgaagagcctgatcaa

SEQ ID NO: 14 - DNA, 21 bp, Unknown
  Description of Unknown REN control target sequence
aggaattataatgcttatcta

SEQ ID NO: 15 - DNA, 1965 bp, Artificial Sequence
  Description of Artificial Sequence Synthetic polynucleotide
tttaattaaggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacagaccgggagctctctggctaactagagaacccactgcttactggcttatcgaaattaatacgactcactatagggagtcccaagctggctagttaagctatcaacaagtttgtacaaaaaagcaggctttaaaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctaagcacttcgtggccgtcgatcgtttaaagggaggtagtga

SEQ ID NO: 16 - DNA, 152 bp, Artificial Sequence
  Description of Artificial Sequence Synthetic polynucleotide
  modified_base (29)..(58): a, c, t, g, unknown or other
  misc_feature (29)..(58): This sequence may encompass 15-30 nucleotides
  modified_base (78)..(107): a, c, t, g, unknown or other
  misc_feature (78)..(107): This sequence may encompass 15-30 nucleotides
  See specification as filed for detailed description of substitutions and preferred embodiments
ctggaggcttgctgaaggctgtatgctgnnnnnnnnnnnnnnnnnnnnnnnnnnnnnngttttggccactgactgacnnnnnnnnnnnnnnnnnnnnnnnnnnnnnncaggacacaaggcctgttactagcactcacatggaacaaatggcc

SEQ ID NO: 17 - DNA, 338 bp, Artificial Sequence
  Description of Artificial Sequence Synthetic polynucleotide
  modified_base (129)..(158): a, c, t, g, unknown or other
  misc_feature (129)..(158): This sequence may encompass 15-30 nucleotides
  modified_base (178)..(207): a, c, t, g, unknown or other
  misc_feature (178)..(207): This sequence may encompass 15-30 nucleotides
  See specification as filed for detailed description of substitutions and preferred embodiments
tgtttgaatgaggcttcagtactttacagaatcgttgcctgcacatcttggaaacacttgctgggattacttcgacttcttaacccaacagaaggctcgagaaggtatattgctgttgacagtgagcgnnnnnnnnnnnnnnnnnnnnnnnnnnnnnntagtgaagccacagatgtannnnnnnnnnnnnnnnnnnnnnnnnnnnnnatgcctactgcctcggacttcaaggggctagaattcgagcaattatcttgtttactaaaactgaataccttgctatctctttgatacatttttacaaagctgaattaaaatggtataaattaaatcacttt

SEQ ID NO: 18 - DNA, 340 bp, Artificial Sequence
  Description of Artificial Sequence Synthetic polynucleotide
  modified_base (130)..(159): a, c, t, g, unknown or other
  misc_feature (130)..(159): This sequence may encompass 15-30 nucleotides
  modified_base (179)..(208): a, c, t, g, unknown or other
  misc_feature (179)..(208): This sequence may encompass 15-30 nucleotides
  See specification as filed for detailed description of substitutions and preferred embodiments
tgtttgaatgaggcttcagtactttacagaatcgttgcctgcacatcttggaaacacttgctgggattacttcttcaggttaacccaacagaaggctaaagaaggtatattgctgttgacagtgagcgcnnnnnnnnnnnnnnnnnnnnnnnnnnnnnntagtgaagccacagatgtannnnnnnnnnnnnnnnnnnnnnnnnnnnnnttgcctactgcctcggacttcaaggggctactttaggagcaattatcttgtttactaaaactgaataccttgctatctctttgatacatttttacaaagctgaattaaaatggtataaattaaatcacttta

SEQ ID NO: 19 - DNA, 340 bp, Artificial Sequence; identical to SEQ ID NO: 18
  Description of Artificial Sequence Synthetic polynucleotide
  modified_base (130)..(159): a, c, t, g, unknown or other
  misc_feature (130)..(159): This sequence may encompass 15-30 nucleotides
  modified_base (179)..(208): a, c, t, g, unknown or other
  misc_feature (179)..(208): This sequence may encompass 15-30 nucleotides
  See specification as filed for detailed description of substitutions and preferred embodiments

SEQ ID NO: 20 - DNA, 152 bp, Artificial Sequence; identical to SEQ ID NO: 16
  Description of Artificial Sequence Synthetic polynucleotide
  modified_base (29)..(58): a, c, t, g, unknown or other
  misc_feature (29)..(58): This sequence may encompass 15-30 nucleotides
  modified_base (78)..(107): a, c, t, g, unknown or other
  misc_feature (78)..(107): This sequence may encompass 15-30 nucleotides
  See specification as filed for detailed description of substitutions and preferred embodiments

SEQ ID NO: 21 - DNA, 152 bp, Artificial Sequence; identical to SEQ ID NO: 16
  Description of Artificial Sequence Synthetic polynucleotide
  modified_base (29)..(58): a, c, t, g, unknown or other
  misc_feature (29)..(58): This sequence may encompass 15-30 nucleotides
  modified_base (78)..(107): a, c, t, g, unknown or other
  misc_feature (78)..(107): This sequence may encompass 15-30 nucleotides
  See specification as filed for detailed description of substitutions and preferred embodiments

SEQ ID NO: 22 - DNA, 132 bp, Artificial Sequence
  Description of Artificial Sequence Synthetic polynucleotide
ctggaggcttgctgaaggctgtatgctgattactttccttctgctcgaagttttggccactgactgacttcgagcaaggaaagtaatcaggacacaaggcctgttactagcactcacatggaacaaatggcc

SEQ ID NO: 23 - DNA, 132 bp, Artificial Sequence
  Description of Artificial Sequence Synthetic polynucleotide
ctggaggcttgctgaaggctgtatgctgatgaacatggaatccatgcaggttttggccactgactgacctgcatggtccatgttcatcaggacacaaggcctgttactagcactcacatggaacaaatggcc

SEQ ID NO: 24 - DNA, 132 bp, Artificial Sequence
  Description of Artificial Sequence Synthetic polynucleotide
ctggaggcttgctgaaggctgtatgctgtactttcttcatttccaccttgttttggccactgactgacaaggtggatgaagaaagtacaggacacaaggcctgttactagcactcacatggaacaaatggcc

SEQ ID NO: 25 - DNA, 132 bp, Artificial Sequence
  Description of Artificial Sequence Synthetic polynucleotide
ctggaggcttgctgaaggctgtatgctgtcaggatacatttctacagctgttttggccactgactgacagctgtagatgtatcctgacaggacacaaggcctgttactagcactcacatggaacaaatggcc

SEQ ID NO: 26 - DNA, 132 bp, Artificial Sequence
  Description of Artificial Sequence Synthetic polynucleotide
ctggaggcttgctgaaggctgtatgctgttatcaggatacatttctacagttttggccactgactgactgtagaaatatcctgataacaggacacaaggcctgttactagcactcacatggaacaaatggcc

SEQ ID NO: 27 - DNA, 132 bp, Artificial Sequence
  Description of Artificial Sequence Synthetic polynucleotide
ctggaggcttgctgaaggctgtatgctgttacagtgtttaatgtttatcgttttggccactgactgacgataaacaaaacactgtaacaggacacaaggcctgttactagcactcacatggaacaaatggcc

SEQ ID NO: 28 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgcttcgagcagaaggaaagtaattagtgaagccacagatgtaattactttccttctgctcgaaatgcctactgcctcggacttcaaggggctagaattcgagcaattatcttgtttactaaaactgaataccttgctatctctttgatacatttttacaaagctgaattaaaatggtataaattaaatcacttt

SEQ ID NO: 29 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgactgcatggattccatgttcattagtgaagccacagatgtaatgaacatggaatccatgcaggtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 30 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgcaaggtggaaatgaagaaagtatagtgaagccacagatgtatactttcttcatttccaccttttgcctactgcctcggacttcaaggggctagaattcgagcaattatcttgtttactaaaactgaataccttgctatctctttgatacatttttacaaagctgaattaaaatggtataaattaaatcacttt

SEQ ID NO: 31 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgcagctgtagaaatgtatcctgatagtgaagccacagatgtatcaggatacatttctacagctatgcctactgcctcggacttcaaggggctagaattcgagcaattatcttgtttactaaaactgaataccttgctatctctttgatacatttttacaaagctgaattaaaatggtataaattaaatcacttt

SEQ ID NO: 32 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgatgtagaaatgtatcctgataatagtgaagccacagatgtattatcaggatacatttctacagtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 33 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgcgataaacattaaacactgtaatagtgaagccacagatgtattacagtgtttaatgtttatcatgcctactgcctcggacttcaaggggctagaattcgagcaattatcttgtttactaaaactgaataccttgctatctctttgatacatttttacaaagctgaattaaaatggtataaattaaatcacttt

SEQ ID NO: 34 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgcttcgagcagaaggaaagtaaatagtgaagccacagatgtatttactttccttctgctcgaaatgcctactgcctcggacttcaaggggctactttaggagcaattatcttgtttactaaaactgaataccttgctatctctttgatacatttttacaaagctgaattaaaatggtataaattaaatcacttta

SEQ ID NO: 35 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgactgcatggattccatgttcattagtgaagccacagatgtaatgaacatggaatccatgcaggtgcctactgcctcggacttcaaggggctactttaggagcaattatcttgtttactaaaactgaataccttgctatctctttgatacatttttacaaagctgaattaaaatggtataaattaaatcacttta

SEQ ID NO: 36 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgcaaggtggaaatgaagaaagtatagtgaagccacagatgtatactttcttcatttccaccttttgcctactgcctcggacttcaaggggctactttaggagcaattatcttgtttactaaaactgaataccttgctatctctttgatacatttttacaaagctgaattaaaatggtataaattaaatcacttta

SEQ ID NO: 37 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgcagctgtagaaatgtatcctgatagtgaagccacagatgtatcaggatacatttctacagctatgcctactgcctcggacttcaaggggctactttaggagcaattatcttgtttactaaaactgaataccttgctatctctttgatacatttttacaaagctgaattaaaatggtataaattaaatcacttta

SEQ ID NO: 38 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgatgtagaaatgtatcctgataatagtgaagccacagatgtattatcaggatacatttctacagtgcctactgcctcggacttcaaggggctactttaggagcaattatcttgtttactaaaactgaataccttgctatctctttgatacatttttacaaagctgaattaaaatggtataaattaaatcacttta

SEQ ID NO: 39 - DNA, 322 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctaaagaaggtatattgctgttgacagtgagcgcgataaacattaaacactgtaatagtgaagccacagatgtattacagtgtttaatgtttatcatgcctactgcctcggacttcaaggggctactttaggagcaattatcttgtttactaaaactgaataccttgctatctctttgatacatttttacaaagctgaattaaaatggtataaattaaatcacttta

SEQ ID NO: 40 - DNA, 1676 bp, Artificial Sequence
  Description of Artificial Sequence Synthetic polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 41 - DNA, 380 bp, Artificial Sequence
  Description of Artificial Sequence Synthetic polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 42 - DNA, 278 bp, Artificial Sequence
  Description of Artificial Sequence Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 43 - DNA, 1017 bp, Artificial Sequence
  Description of Artificial Sequence Synthetic polynucleotide
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 44 - DNA, 589 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 45 - DNA, 477 bp, Artificial Sequence
  Description of Artificial Sequence Synthetic polynucleotide
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 46 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 47 - DNA, 652 bp, Mus musculus
cgcggtcctttcctgcggcgccttccgtccgtcggcttctcgtcttgctctctctggtccctccggaggaggccgccgcgcgtctcccggggaagcatggcgatgaaagcggtgtgcgtgctgaagggcgacggtccggtgcagggaaccatccacttcgagcagaaggcaagcggtgaaccagttgtgttgtcaggacaaattacaggattaactgaaggccagcatgggttccacgtccatcagtatggggacaatacacaaggctgtaccagtgcaggacctcattttaatcctcactctaagaaacatggtggcccggcggatgaagagaggcatgttggagacctgggcaatgtgactgctggaaaggacggtgtggccaatgtgtccattgaagatcgtgtgatctcactctcaggagagcattccatcattggccgtacaatggtggtccatgagaaacaagatgacttgggcaaaggtggaaatgaagaaagtacaaagactggaaatgctgggagccgcttggcctgtggagtgattgggattgcgcagtaaacattccctgtgtggtctgagtctcagactcatctgctaccctcaaaccattaaactgtaatctgaagagttgtaaaaaaaaaaaaaaaaa

SEQ ID NO: 48 - DNA, 2039 bp, Macaca fascicularis
ttttgcggcatagtctcctgcagcgtttgcggtcagtctcgcaatattcggaagcaggaccgcggcgtggcctagcaagtcatggcgatgaaggccgtgtgcgtgttgaagggcgacagcccagtgcagggcaccatcaatttcgagcagaaggaaagtaatggaccagtgaaggtgtggggaagcattacaggattgactgaaggcctgcatggataccatgttcatcagtttggagataatacacaaggctgtaccagtgcaggtcctcactttaatcctctatccagacaacacggtgggccaaaggatgaagagaggcatgttggagacctgggcaatgtgactgctggcaaagatggtgtggccaaggtgtctttcgaagattctgtgatctcgctctcaggagaccattccatcattggccgcacattggtggtccatgaaaaagcagatgacttgggcaaaggtggaaatgaagaaagtaaaaagacaggaaacgctggaggtcgtctggcttgtggtgtaattgggatcgcccattaaacattcccttggatgtagtctgaggcccattaactcatctgttatcctgctagctgtagaaatgtatcttgataaacattaaacactgtaatcttaagagtgtaattgtgtgacgtttgcttagtacctgtaatgagaaactggttgatgatcacttggaagatttgtatagttttataaaactcaattaaaatgtctgtttcaatgacctgtattttgccagacttaatcacagatgggtattaaacttgtcggacacatcttcctcctccccacccgagcctggagcactctaacccttggagaccccctaagccctgttcctccagagaccgaggccctccagaagggctgagcggggataggcttgcctgagcctggagctgggctttggggcagcctgcgaccctccccacttgtgccccttctcctgggatctctgtgtcttcccttttctttctggggccaggaagtcagcgtcaactcctaggccccagatgcaggggcccggaaacacctgctctcccctgagccccaaatgcaggggcctgggaacaccgtgctgtcacctgagcctgggggtcccatcccaggaagaggggctgtctcaggacctgagtcctcaggggccccgcacattcaatctgaaggtgaccctggcctggccgaagctggaagagccgtggggacgcagccagtaaacagagcgtaaggctcaggtgctggttggttaatccgtttctggaggaagagtatgacccccacctgtgatggggtccttgtgtggtggggaccggggccagtgggctccagaccgcatgcttaacccgtggatgtgaaacctgcagcagagaaggaaggtcgcatgagtcagatcccagtccagtagtcagtggagggtgagggtgaccccatctgctatttttgtgcccatcctcagacagccatttggggatgtgcctattagggctccctaagaactcagatgcccaggaagcccagcccctcaggacgtacccacacgcagccttcccttgacgcctacgtttctgggcacatgaggcatctttcctggaaccccgagccagccctgtcccgccccaacgcagcatggcactcaggagatacaggctggatgtggggcggtccttctggggaggcctggcctagcagcctgccctctgcacgctgcccacctgagccctccctgccaggcttcatgctggggtgggccacatgccaggacaagaggaccccagcagaaagccagccccggactcacttgggtgtgttaaaatggcttctacctacatacaacatggtaaaaggtgtggaacgtttgcttgaaaataattgggggtgggggagtggtgagagggtggggatgggagggttcctggaattggttctttatcctgattagatgtgaaggcactaatgctgatttctagtagtaaaaagagcaccaatagtcaaaaaaaaaaaaaaaaaaa

SEQ ID NO: 49 - DNA, 860 bp, Callithrix jacchus
gagcgcgcgcagggcgattggctccgggccagagtgggtggtgcacgtaggtccggcctataaagtgcccgcggcgctctcgcttgggtttgcgccgttctcttctgcagcgtctgtggtttctctggcagtcgttggaacccggatccaggcgtggcctcgcgagtgatggcgatgaaggcggtgtgcgtgttgaagggcgacggcccggtgcagggcaccatcaatttcgagcagaaggaaagtaatggaccagttaaggtgtggggaagcattacaggattggctgaaggcctgcatggattccatgttcatcagtttggagacaacacacaaggctgtaccagtgcaggtcctcactttaatcctctatccagaaaacatggtgggccagaggatgaagagaggcatgttggagacctgggcaatgtgactgctggtaaagatggtgtggccagtgtgtcaattgaagattctgtgatctcactctcaggagtccattccatcattggccgcacgttggtggtccatgaaaaagcagatgacttgggcaaaggtggaaatgaagaaagtacaaagacaggaaacgctggaagtcgtttggcttgtggtgtcattgggatcgcccagtaaacattgccctggatgtagtctgagtcccattaactcatctgttatcctggctagctgtagaaatgtaacttgacattaaacactgtaatcttaaaagcgtcattttaagtgtgattttgaaaaaaaaagttgctttaaagtacctctaatgagaaactggtttatgatcacttggaagatttgtatagttttataaacctcacattaaaatgtttcagtgacctgta

SEQ ID NO: 50 - DNA, 465 bp, Macaca mulatta
atggcgatgaaggccgtgtgcgtgttgaagggcgacagcccagtgcagggcaccatcaatttcgagcagaaggaaagtaatggaccagtgaaggtgtggggaagcattacaggattgactgaaggcctgcatggattccatgttcatcagtttggagataatacacaaggctgtaccagtgcaggtcctcactttaatcctctatccagacaacacggtgggccaaaggatgaagagaggcatgttggagacctgggcaatgtgactgctggcaaagatggtgtggccaaggtgtctttcgaagattctgtgatctcgctctcaggagaccattccatcattggccgcacattggtggtccatgaaaaagcagatgacttgggcaaaggtggaaatgaagaaagtaaaaagacaggaaacgctggaggtcgtctggcttgtggtgtaattgggatcgcccaataa

SEQ ID NO: 51 - DNA, 3255 bp, Artificial Sequence
  Description of Artificial Sequence Synthetic polynucleotide
gcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtacctgtttgaatgaggcttcagtactttacagaatcgttgcctgcacatcttggaaacacttgctgggattacttcttcaggttaacccaacagaaggctaaagaaggtatattgctgttgacagtgagcgcaaggtggaaatgaagaaagtatagtgaagccacagatgtatactttcttcatttccaccttttgcctactgcctcggacttcaaggggctactttaggagcaattatcttgtttactaaaactgaataccttgctatctctttgatacatttttacaaagctgaattaaaatggtataaattaaatcactttagcctggaggcttgctgaaggctgtatgctgtcaggatacatttctacagctgttttggccactgactgacagctgtagatgtatcctgacaggacacaaggcctgttactagcactcacatggaacaaatggccgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgc

SEQ ID NO: 52 - DNA, 3259 bp, Artificial Sequence
  Description of Artificial Sequence Synthetic polynucleotide
gcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtaccctggaggcttgctgaaggctgtatgctgattactttccttctgctcgaagttttggccactgactgacttcgagcaaggaaagtaatcaggacacaaggcctgttactagcactcacatggaacaaatggccggtacctgtttgaatgaggcttcagtactttacagaatcgttgcctgcacatcttggaaacacttgctgggattacttcttcaggttaacccaacagaaggctaaagaaggtatattgctgttgacagtgagcgcaaggtggaaatgaagaaagtatagtgaagccacagatgtatactttcttcatttccaccttttgcctactgcctcggacttcaaggggctactttaggagcaattatcttgtttactaaaactgaataccttgctatctctttgatacatttttacaaagctgaattaaaatggtataaattaaatcactttagagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgc

SEQ ID NO: 53 - DNA, 3259 bp, Artificial Sequence
  Description of Artificial Sequence Synthetic polynucleotide
gcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtaccctggaggcttgctgaaggctgtatgctgattactttccttctgctcgaagttttggccactgactgacttcgagcaaggaaagtaatcaggacacaaggcctgttactagcactcacatggaacaaatggccggtacctgtttgaatgaggcttcagtactttacagaatcgttgcctgcacatcttggaaacacttgctgggattacttcgacttcttaacccaacagaaggctcgagaaggtatattgctgttgacagtgagcgcagctgtagaaatgtatcctgatagtgaagccacagatgtatcaggatacatttctacagctatgcctactgcctcggacttcaaggggctagaattcgagcaattatcttgtttactaaaactgaataccttgctatctctttgatacatttttacaaagctgaattaaaatggtataaattaaatcactttgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcgctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgc

SEQ ID NO: 54 - DNA, 711 bp, Artificial Sequence
  Description of Artificial Sequence Synthetic polynucleotide
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 55 - DNA, 11671 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtacctgtttgaatgaggcttcagtactttacagaatcgttgcctgcacatcttggaaacacttgctgggattacttcttcaggttaacccaacagaaggctaaagaaggtatattgctgttgacagtgagcgcaaggtggaaatgaagaaagtatagtgaagccacagatgtatactttcttcatttccaccttttgcctactgcctcggacttcaaggggctactttaggagcaattatcttgtttactaaaactgaataccttgctatctctttgatacatttttacaaagctgaattaaaatggtataaattaaatcactttagcctggaggcttgctgaaggctgtatgctgtcaggatacatttctacagctgttttggccactgactgacagctgtagatgtatcctgacaggacacaaggcctgttactagcactcacatggaacaaatggccgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccaaggcctgctggtaatcaattgcctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccaattgctggtcaccatcctgtcggctgtggcacaggctgaacgccggaggatcaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 56 - DNA, 11675 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtaccctggaggcttgctgaaggctgtatgctgattactttccttctgctcgaagttttggccactgactgacttcgagcaaggaaagtaatcaggacacaaggcctgttactagcactcacatggaacaaatggccggtacctgtttgaatgaggcttcagtactttacagaatcgttgcctgcacatcttggaaacacttgctgggattacttcttcaggttaacccaacagaaggctaaagaaggtatattgctgttgacagtgagcgcaaggtggaaatgaagaaagtatagtgaagccacagatgtatactttcttcatttccaccttttgcctactgcctcggacttcaaggggctactttaggagcaattatcttgtttactaaaactgaataccttgctatctctttgatacatttttacaaagctgaattaaaatggtataaattaaatcactttagagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccaaggcctgctggtaatcaattgcctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccaattgctggtcaccatcctgtcggctgtggcacaggctgaacgccggaggatcaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 57 - DNA, 11674 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggctagcggtaccctggaggcttgctgaaggctgtatgctgattactttccttctgctcgaagttttggccactgactgacttcgagcaaggaaagtaatcaggacacaaggcctgttactagcactcacatggaacaaatggccggtacctgtttgaatgaggcttcagtactttacagaatcgttgcctgcacatcttggaaacacttgctgggattacttcgacttcttaacccaacagaaggctcgagaaggtatattgctgttgacagtgagcgcagctgtagaaatgtatcctgatagtgaagccacagatgtatcaggatacatttctacagctatgcctactgcctcggacttcaaggggctagaattcgagcaattatcttgtttactaaaactgaataccttgctatctctttgatacatttttacaaagctgaattaaaatggtataaattaaatcactttgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcacgcctaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccaaggcctgctggtaatcaattgcctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccaattgctggtcaccatcctgtcggctgtggcacaggctgaacgccggaggatcaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 58 - DNA, 225 bp, Artificial Sequence
  Description of Artificial Sequence Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggtgcaggacagcaagggggaggattgggaagacaatagcaggcatgctggggatgcggtgggctctatgg

SEQ ID NO: 59 - DNA, 135 bp, Artificial Sequence
  Description of Artificial Sequence Synthetic polynucleotide
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 60 - DNA, 557 bp, Artificial Sequence
  Description of Artificial Sequence Synthetic polynucleotide
attcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaa

SEQ ID NO: 61 - DNA, 395 bp, Artificial Sequence
  Description of Artificial Sequence Synthetic polynucleotide
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 62 - DNA, 49 bp, Artificial Sequence
  Description of Artificial Sequence Synthetic oligonucleotide
aataaaatatctttattttcattacatctgtgtgttggttttttgtgtg

SEQ ID NO: 63 - DNA, 50 bp, Artificial Sequence
  Description of Artificial Sequence Synthetic oligonucleotide
cggcaataaaaagacagaataaaacgcacgggtgttgggtcgtttgttca

SEQ ID NO: 64 - DNA, 141 bp, Artificial Sequence
  Description of Artificial Sequence Synthetic polynucleotide
aataaaatatctttattttcattacatctgtgtgttggttttttgtgtgaacatacgctctccatcaaaacaaaacgaaacaaaacaaactagcaaaataggctgtccccagtgcaagtgcaggtgccagaacatttctct

SEQ ID NO: 65 - DNA, 132 bp, Artificial Sequence
  Description of Artificial Sequence Synthetic polynucleotide
  modified_base (29)..(49): a, c, t, g, unknown or other
  modified_base (69)..(87): a, c, t, g, unknown or other
ctggaggcttgctgaaggctgtatgctgnnnnnnnnnnnnnnnnnnnnngttttggccactgactgacnnnnnnnnnnnnnnnnnnncaggacacaaggcctgttactagcactcacatggaacaaatggcc

SEQ ID NO: 66 - DNA, 321 bp, Artificial Sequence
  Description of Artificial Sequence Synthetic polynucleotide
  modified_base (130)..(150): a, c, t, g, unknown or other
  modified_base (170)..(191): a, c, t, g, unknown or other
tgtttgaatgaggcttcagtactttacagaatcgttgcctgcacatcttggaaacacttgctgggattacttcgacttcttaacccaacagaaggctcgagaaggtatattgctgttgacagtgagcgmnnnnnnnnnnnnnnnnnnnnntagtgaagccacagatgtannnnnnnnnnnnnnnnnnnnnntgcctactgcctcggacttcaaggggctagaattcgagcaattatcttgtttactaaaactgaataccttgctatctctttgatacatttttacaaagctgaattaaaatggtataaattaaatcacttt

SEQ ID NO: 67 - DNA, 322 bp, Artificial Sequence
  Description of Artificial Sequence Synthetic polynucleotide
  modified_base (130)..(150): a, c, t, g, unknown or other
  modified_base (170)..(191): a, c, t, g, unknown or other
tgtttgaatgaggcttcagtactttacagaatcgttgcctgcacatcttggaaacacttgctgggattacttcttcaggttaacccaacagaaggctaaagaaggtatattgctgttgacagtgagcgmnnnnnnnnnnnnnnnnnnnnntagtgaagccacagatgtannnnnnnnnnnnnnnnnnnnnntgcctactgcctcggacttcaaggggctactttaggagcaattatcttgtttactaaaactgaataccttgctatctctttgatacatttttacaaagctgaattaaaatggtataaattaaatcacttta

SEQ ID NO: 68 - DNA, 608 bp, Artificial Sequence
  Description of Artificial Sequence Synthetic polynucleotide
agctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatc

SEQ ID NO: 69 - DNA, 152 bp, Artificial Sequence; identical to SEQ ID NO: 16
  Description of Artificial Sequence Synthetic polynucleotide
  modified_base (29)..(58): a, c, t, g, unknown or other
  misc_feature (29)..(58): This sequence may encompass 15-30 nucleotides
  modified_base (78)..(107): a, c, t, g, unknown or other
  misc_feature (78)..(107): This sequence may encompass 15-30 nucleotides
  See specification as filed for detailed description of substitutions and preferred embodiments

SEQ ID NO: 70 - DNA, 340 bp, Artificial Sequence; identical to SEQ ID NO: 18
  Description of Artificial Sequence Synthetic polynucleotide
  modified_base (130)..(159): a, c, t, g, unknown or other
  misc_feature (130)..(159): This sequence may encompass 15-30 nucleotides
  modified_base (179)..(208): a, c, t, g, unknown or other
  misc_feature (179)..(208): This sequence may encompass 15-30 nucleotides
  See specification as filed for detailed description of substitutions and preferred embodiments

SEQ ID NO: 71 - DNA, 338 bp, Artificial Sequence; identical to SEQ ID NO: 17
  Description of Artificial Sequence Synthetic polynucleotide
  modified_base (129)..(158): a, c, t, g, unknown or other
  misc_feature (129)..(158): This sequence may encompass 15-30 nucleotides
  modified_base (178)..(207): a, c, t, g, unknown or other
  misc_feature (178)..(207): This sequence may encompass 15-30 nucleotides
  See specification as filed for detailed description of substitutions and preferred embodiments